(19) United States
(12) Patent Application Publication
(10) Pub. No.: US 2026/0295020 A1
(43) Pub. Date: Oct. 1, 2026

(54) ANIMAL DIET COMPRISING PHYTASE, THEREBY OBVIATING THE NEED FOR MINERAL SUPPLEMENTATION

(71) Applicant: INTERNATIONAL N&H DENMARK APS, Kongens Lyngby (DK)

(72) Inventors: Leon Marchal, Oegstgeest (NL); Abiodun Bello, Rochester, NY (US); Yueming Dersjant-Li, Oegstgeest (NL); Kirsty Gibbs, Wiltshire (GB); Karsten Matthias Kragh, Kongens Lyngby (DK); Charlotte Horsmans Poulsen, Kongens Lyngby (DK)

(21) Appl. No.: 19/477,703

(22) PCT Filed: Apr. 24, 2024
(86) PCT No.: PCT/US2024/026049

§ 371 Date: Oct. 22, 2025

Related U.S. Application Data
(60) Provisional application No. 63/498,065, filed on Apr. 25, 2023.
(60) Provisional application No. 63/520,797, filed on Aug. 21, 2023.

(51) Int. Cl. A61K 38/46; A23K 10/30; A23K 20/142; A23K 20/189; A23K 20/22; A23K 20/24; A23K 50/30; A23K 50/60; A23K 50/75; A61P 3/00; A61P 31/04

(52) U.S. Cl. CPC A61K 38/465; A23K 10/30; A23K 20/142; A23K 20/189; A23K 20/22; A23K 20/24; A23K 50/30; A23K 50/60; A23K 50/75; A61P 3/00; A61P 31/04; C12Y 301/03026

(57) ABSTRACT

Provided herein are animal diets containing phytase polypeptides or fragments thereof wherein the diet contains no or substantially no exogenously added trace minerals.

### CROSS-REFERENCE TO RELATED APPLICATIONS

This application claims priority to U.S. Provisional Patent Application No. 63/498,065, filed Apr. 25, 2023 and U.S. Provisional Patent Application No. 63/520,797, filed Aug. 21, 2023, the disclosure of each of which is incorporated by reference herein in its entirety.

### INCORPORATION BY REFERENCE

The sequence listing provided in the file named 20240419_NB42195-WO-PCT_Seq List.xml with a size of 64 KB which was created on Apr. 22, 2024 and which is filed herewith, is incorporated by reference herein in its entirety.

### FIELD

The field pertains to animal diets containing no or substantially no or decreased exogenously added trace minerals as well as engineered phytase polypeptides and uses of the same for enhancing animal performance.

### BACKGROUND

Trace minerals (TM) are essential in animal diets for the healthy development of bones, enzyme structure and function and for catalyzing biological reactions (Suttle, 2010). The amounts of key TM in raw materials and ingredients commonly used in commercial broiler diets are considered to be insufficient (due to low availability) or too variable to support optimal growth and development.

Certainly, there is evidence that the withdrawal of supplemental TM (in particular zinc) from day 1 results in poor growth and leg problems (Bao et al., 2007, 2009, 2010; Singh et al. 2015). Hence, it is common practice to supplement diets with zinc (Zn), iron (Fe), copper (Cu), manganese (Mn) and selenium (Se), either from inorganic or organic sources.

Exogenous microbial phytases are added to broiler diets as an effective means of increasing the availability of phosphorus (P). Phytase efficacy in broilers has been extensively researched and is well accepted (Selle and Ravindran, 2007; Humer et al. 2015). The substrate of phytase, phytate (IP6, myo-inositol hexakisphosphate) is a potent antinutrient that has a strong affinity for binding with mineral cations (Ca²⁺, Zn²⁺, Fc²⁺, Mn²⁺, Fc³⁺, Mg²⁺, Cu²⁺, Co²⁺) in the neutral pH environment of the small intestine (Selle and Ravindran, 2007; Selle et al., 2009). These complexes are insoluble at neutral pH, reducing the availability of both phytate and the bound mineral or TM to the animal (Selle et al., 2009). If phytase is active in the early gastrointestinal tract (GIT) and can break down phytate quickly and completely, it should reduce the binding of phytate to TM and improve TM bioavailability. A novel consensus bacterial 6-phytase variant with high efficacy in broilers to degrade phytate and release P in the early GIT and to improve the digestibility and utilization of energy, P, crude protein, and amino acids has recently been developed and commercialized (Dersjant-Li et al., 2020; Christensen et al., 2020; Dersjant-Li et al., 2021; Dersjant-Li et al., 2022).

Accordingly, a need exists to determine the extent to which the novel phytase could “replace” exogenously added TM in a commercial animal diet over an entire growth cycle, while still ensuring normal growth performance and TM utilization at tissue level.

### SUMMARY

Provided herein, inter alia, are animal diets containing phytases which are free or substantially free of exogenously added trace minerals. When fed to animals (such as, without limitation, poultry), these diets ensure normal growth during all phases of development compared to diets which contain trace mineral supplementation.

Accordingly, in one aspect, provided herein an animal diet comprising (i) an engineered phytase polypeptide or a fragment thereof comprising phytase activity; and (ii) lacking one or more exogenously added trace minerals. In some embodiments, the one or more trace mineral is one or more trace mineral selected from the group consisting of zinc (Zn), iron (Fe), copper (Cu), manganese (Mn) and selenium (Se). In some embodiments of any of the embodiments disclosed herein, the phytase polypeptide or a fragment thereof comprising phytase activity comprises at least 82% sequence identity with the amino acid sequence set forth in SEQ ID NO:1. In some embodiments of any of the embodiments disclosed herein, said phytase polypeptide comprises an amino acid sequence selected from the group consisting of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:16, SEQ ID NO: 17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID: NO: 33, SEQ ID NO: 34, SEQ ID NO:35, SEQ ID NO:36, and SEQ ID NO:37. In some embodiments of any of the embodiments disclosed herein, the diet further comprises one or more of (a) a direct fed microbial comprising at least one bacterial strain, (b) at least one other enzyme, and/or (c) an essential oil In some embodiments of any of the embodiments disclosed herein, the engineered phytase polypeptide or fragment thereof is present in an amount of at least about 0.1 g/ton feed.

In some embodiments of any of the embodiments disclosed herein, the diet comprises calcium from about 0.62 to 0.72% in a starter diet, about 0.54 to 0.64% in a grower diet, and/or about 0.42 to 0.55% in a finisher diet. In some embodiments of any of the embodiments disclosed herein, the finisher diet comprises about 0.46 to 0.55% or from about 0.42 to 0.50% calcium. In some embodiments of any of the embodiments disclosed herein, the diet comprises amino acids from about 1.18 to 1.22% in a starter diet, about 1.06 to 1.10% in a grower diet, and/or about 0.88 to 1% in finisher diet. In some embodiments of any of the embodiments disclosed herein, the finisher diet comprises about 0.96 to 1.0% or from about 0.88 to 0.92% amino acids. In some embodiments of any of the embodiments disclosed herein, the amino acids comprise digestible lysine. In some embodiments of any of the embodiments disclosed herein, the diet comprises metabolizable energy from about 2824 to 2950 kcal/kg in a starter diet, about 2924 to 3050 kcal/kg in a grower diet, and/or about 2970 to 3120 kcal/kg in a finisher diet. In some embodiments, the finisher diet comprises about 2970 to 3100 kcal/kg or from about 2994 to 3120 kcal/kg metabolizable energy. In some embodiments of any of the embodiments disclosed herein, the diet comprises sodium from about 0.13 to 0.17%. In some embodiments of any of the embodiments disclosed herein, the phytase is present at a dose of between about 500 FTU/kg to about 2000 FTU/kg (such as any of about 500 FTU/kg, 550 FTU/kg, 600 FTU/kg, 650 FTU/kg, 700 FTU/kg, 750 FTU/kg, 800 FTU/kg, 850 FTU/kg, 900 FTU/kg, 950 FTU/kg, 1000 FTU/kg, 1050 FTU/kg, 1100 FTU/kg, 1150 FTU/kg, 1200 FTU/kg, 1250 FTU/kg, 1300 FTU/kg, 1350 FTU/kg, 1400 FTU/kg, 1450 FTU/kg, 1500 FTU/kg, 1550 FTU/kg, 1600 FTU/kg, 1650 FTU/kg, 1700 FTU/kg, 1750 FTU/kg, 1800 FTU/kg, 1850 FTU/kg, 1900 FTU/kg, 1950 FTU/kg, or 2000 FTU/kg or more). In some embodiments of any of the embodiments disclosed herein, the diet contains a phytate source comprising one or more of corn, wheat, soybean meal, rapeseed, rice and/or wheat bran. In some embodiments of any of the embodiments disclosed herein, the diet further comprises oat hulls. In some embodiments of any of the embodiments disclosed herein, the diet lacks meat and/or bone meal. In some embodiments of any of the embodiments disclosed herein, the diet further comprises one or more additional feed enzymes selected from the group consisting of a xylanase, a protease, an amylase, and a glucoamylase. In some embodiments of any of the embodiments disclosed herein, the diet is a starter diet. In some embodiments of any of the embodiments disclosed herein, the diet is a grower diet. In some embodiments of any of the embodiments disclosed herein, the diet is a finisher diet. In some embodiments of any of the embodiments disclosed herein, the animal is poultry. In some embodiments of any of the embodiments disclosed herein, the animal is swine. In some embodiments of any of the embodiments disclosed herein, the animal is a ruminant. In some embodiments, the ruminant is a calf.

In additional aspects, provided herein is a method for improving animal performance on one or more metrics comprising administering an effective amount of any of the animal diets disclosed herein to an animal. In some embodiments of any of the embodiments disclosed herein, the one or more metrics is selected from the group consisting of increased feed efficiency, increased weight gain, reduced feed conversion ratio, improved digestibility of nutrients or energy in a feed, improved nitrogen retention, improved ability to avoid the negative effects of necrotic enteritis, and improved immune response. In some embodiments of any of the embodiments disclosed herein, the animal is poultry, swine, or a ruminant animal. In some embodiments, the poultry is selected from the group consisting of turkeys, ducks, chickens, geese, pheasants, quail, and emus. In some embodiments, the chicken is selected from the group consisting of layers and broilers.

In yet further aspects, provided herein is a method for reducing pathogenic bacteria populations in animals comprising administering an effective amount of any of the animal diets disclosed herein to an animal. In some embodiments, a) the one or more trace mineral is iron (Fe); and/or b) the diet further contains no or substantially no exogenously-added inorganic phosphate. In some embodiments of any of the embodiments disclosed herein, the pathogenic bacterial population is one or more bacteria selected from the group consisting of Actinobacillus, Bordetalla, Campylobacter (e.g., C. jejuni), Clostridium, Corynebacterium, Escherichia coli (e.g., enterotoxigenic Escherichia coli (ETEC)), Globicatella, Listeria, Mycobacterium, Salmonella, Staphylococcus, and Streptococcus. In some embodiments of any of the embodiments disclosed herein, the animal is poultry, swine, or a ruminant animal. In some embodiments, the poultry is selected from the group consisting of turkeys, ducks, chickens, geese, pheasants, quail, and emus. In some embodiments, the chicken is selected from the group consisting of layers and broilers. In some embodiments, the ruminant is a calf.

### DETAILED DESCRIPTION

There is a growing global sustainability awareness regarding the proper use of finite resources like trace minerals and pollution resulting from use of the same. While commercially farmed animals such as poultry have among the lowest environmental impact of all animal proteins, in commercial practice, it still uses significant amounts of exogenously added trace minerals in diets. The inventors of the present application have surprisingly discovered that use of next generation biosynthetic bacterial 6-phytases in broiler diets can completely eliminate the need to supplement the diet with one or more trace minerals and/or can substantially reduce the need to supplement the diet with one or more sources of trace minerals. All animals fed phytase-supplemented low or trace mineral-free diets had comparable or better growth and feed conversion versus animals fed a diet containing one or more exogenously added trace minerals. Moreover, testing showed that these animals did not exhibit symptoms of bone loss or demineralization characteristic of animals experiencing trace mineral deficiency. Rather, bone breaking strength of animals fed phytase supplemented with exogenously added trace-free diets did not deteriorate compared to the controls. This represents the first report to show a 100% vegetable total trace mineral free and commercially relevant broiler diet with normal growth characteristics in all growth phases. Consequently, use of the phytase supplemented trace mineral-free diets disclosed herein provides both an economic advantage in the form of decreased costs of feed (particularly for large-scale industrial-sized flocks) as well as a significant environmental benefit due to decreased pollution as a byproduct of large-scale animal production.

In addition, the inventors of the present invention have surprisingly discovered that use of next generation biosynthetic bacterial 6-phytases in combination with animal dicts low in trace mineral content can decrease the pathogenic bacterial burden (such as, without limitation, Campylobacter spp., e.g., C. jejuni) that is commonly seen associated with particular animal-based food sources (for example, poultry).

All patents, patent applications, and publications cited are incorporated herein by reference for all purposes in their entirety.

In this disclosure, many terms and abbreviations are used. The following definitions apply unless specifically stated otherwise.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The term “and/or” and “or” are used interchangeably herein and refer to a specific disclosure of each of the two specified features or components with or without the other. Thus, the term “and/or” as used in a phrase such “A and/or B” herein is intended to include “A and B,” “A or B,” “A” (alone), and “B” alone. Likewise, the term “and/or” as used a phrase such as “A, B and/or C” is intended to encompass each of the following aspects: A, B and C; A, B or C; A or C; A or B; B or C; A and C; A and B; B and C; A (alone); B (alone); and C (alone).

Words using the singular include the plural, and vice versa.

The terms “comprises,” “comprising,” “includes,” “including,” “having” and their conjugates are used interchangeably and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a methods, and those additional materials or steps that do not materially affect the basic characteristics of the material or method.

Throughout this application, various embodiments can be presented in a range format. It should be understood that the description in range format is merely for convenience and brevity and should not be construed as an inflexible limitation on the scope of the embodiments described herein. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4 and from 1 to 5, from 2 to 3, from 2 to 4, from 2 to 5, from 2 to 6, from 3 to 4, from 3 to 5, from 3 to 6, etc. as well as individual numbers within that range, for example, 1, 2, 3, 4, 5 and 6. This applies regardless of the breadth of the range.

The term “about” as used herein can allow for a degree of variability in a value or range, for example, within 10%, within 5%, or within 1% of a stated value or of a stated limit of a range.

The term “phytase” (myo-inositol hexakisphosphate phosphohydrolase) refers to a class of phosphatase enzymes that catalyzes the hydrolysis of phytic acid (myo-inositol hexakisphosphate or IP6)—an indigestible, organic form of phosphorus that is found in grains and oil seeds—and releases a usable form of inorganic phosphorus.

The terms “animal” and “subject” are used interchangeably herein and refer to any organism belonging to the kingdom Animalia and includes, without limitation, mammals (excluding humans), non-human animals, domestic animals, livestock, farm animals, zoo animals, breeding stock and the like. For example, there can be mentioned all non-ruminant and ruminant animals. In an embodiment, the animal is a non-ruminant, i.e., mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment, the animal is a ruminant animal including, but not limited to, cattle, young calves, goats, sheep, giraffes, bison, moose, elk, yaks, water buffalo, deer, camels, alpacas, llamas, antelope, pronghorn and nilgai.

“Poultry” as used herein refers to birds such as turkeys, pheasants, geese, emus, ducks, chicken, broiler chicks, and layers.

The reference book “Commercial Poultry Nutrition” (3rd edition, 2005, ISBN 0-9695600 May 2; incorporated by reference herein) is a standard textbook relating to the area of nutritional aspects of chicken production. Below is a summary considered relevant as a background for the present invention.

The major ingredients delivering energy in poultry diets are corn, soybean, soy oil and amino acids. Corn is a major contributor of metabolizable energy. The starch of the endosperm, which is mainly composed of amylopectin, and the germ which is mostly oil constitute the energy value of corn. Typical energy values of corn alone expressed in kcal/kg at 85% dry matter are ranging from 3014 to 3313. Energy levels of starter and grower diets are typically in the range of 3000 to 3100 Kcal/kg. In many countries, wheat is also commonly used in poultry diets. Such diets have similar energy levels as mentioned above. As a protein source soybean has become the worldwide standard against which other protein sources are compared. Its amino acid profile is excellent for most types of poultry, and when combined with corn or sorghum, methionine is usually the only factor in inadequate amounts. Additionally, fats and oils provide a concentrated source of energy in the diets and even relatively small changes in levels can have significant effects on diet ME. Finally, the diet is supplemented with synthetic amino acids such as methionine and lysine. Other important sources used are barley, sorghum and other cereals, and byproducts contributing to energy.

Chickens used in optimized commercial broiler production are typically fed different diets depending upon their age. For example, chickens for broiler production may be raised using three or four diets. These diets are typically called a “starter”, “grower” and “finisher”. “Pre-starter” diets are also possible. According, the engineered phytases disclosed herein may be included in a starter diet only, a grower diet only, a finisher diet only, a combination of any two or a combination of all phases at different dose levels—as long as they contain no or substantially no exogenously added trace minerals.

“Trace minerals,” as used herein, refer to essential minerals found in minute amounts in animal feed and which relate to organic molecules, such as polysaccharides, amino acids, and as co-factors for enzyme function. Non-limiting examples of trace minerals include one or more (such as 1, 2, 3, 4, or 5) zinc (Zn), iron (Fe), copper (Cu), manganese (Mn) and selenium (Se). The animal diets disclosed herein, contain no or substantially no exogenously added trace minerals (such as ZnSO₄ or ZnO or Zinc methionine or other organic forms or other forms of trace mineral premixes) when said diets also include the engineered phytase polypeptides disclosed herein at proper dose.

“Inorganic phosphorous” and “inorganic phosphate” are used interchangeably herein to denote dietary supplements commonly added to poultry feed to ensure the animal receives sufficient phosphate to satisfy the nutritional requirements of an animal. The poultry diets disclosed herein can additionally in some non-limiting embodiments contain no or substantially no inorganic phosphate when said diets also include the engineered phytase polypeptides disclosed herein at proper dose with diets containing sufficient phytate as substrate.

The expression “substantially none” or “substantially no” as used herein to describe the amount of trace minerals and/or inorganic phosphate in the diet formulations disclosed herein, means that any amounts that are present are either trace amounts, amounts included unintentionally, and/or amounts that that are less than about 0.1% in the diets.

The “starter”, “grower” and “finisher” diets are typically distinguished by crude protein content, which is often provided by ingredients such as soybean meal (SBM). For example, a starter diet for a broiler chicken may optionally contain crude protein contents of around 22-25% by weight, such as 22%, 23%, 24% or 25%, with 23 or 25% being preferred. In a further example, a grower diet for a broiler chicken may optionally contain crude protein contents of around 21-23% by weight, such as 21%, 22% or 23%, with 22% being preferred. In a further example, a finisher diet for a broiler chicken may optionally contain crude protein contents of around 19-23% by weight, such as 19%, 20%, 21%, 22% or 23%, with 19%, 20%, or 21% being preferred.

Additionally or alternatively, the “starter”, “grower” and “finisher” may be distinguished by metabolizable energy (ME) content, which is typically lowest for the starter diet and highest for the finisher diet, with the grower diet having a level between the two. For example, a starter diet for a broiler chicken may have an ME of about 3000 or 3025 kcal/kg (±50, 40, 30, 20, 10, 5 or less kcal/kg). In a further example, a grower diet for a broiler chicken may have an ME of about 3100 or 3150 kcal/kg (±50, 40, 30, 20, 10, 5 or less kcal/kg). In a further example, a grower diet for a broiler chicken may have an ME of about 3200 kcal/kg (±50, 40, 30, 20, 10, 5 or less kcal/kg).

The animal diets described herein may either be a vegetarian or non-vegetarian product. A vegetarian product contains no meat or fish products. A non-vegetarian dict may contain either, or both, fish product (such as fish meal) or meat product (such as meat derivatives or other non-inorganic phosphate-containing and/or non-trace mineral-containing meat products).

The terms “mixer liquid application” and “MLA” are used interchangeably herein and refer to animal feed production wherein heat sensitive compounds, specifically, enzymes can be applied in a liquid form to animal feed prior to conditioning and pelleting and remain functional in the feed after conditioning and pelleting.

The terms “feed,” an “animal feed,” or “diet” are used interchangeably herein to mean any natural or artificial diet, meal or the like or components of such meals intended or suitable for being eaten, taken in, digested, by a non-human animal, respectively. Preferably term “feed” is used with reference to products that are fed to animals in the rearing of livestock.

A “feed additive” as used herein refers to one or more ingredients, products of substances (e.g., cells), used alone or together, in nutrition (e.g., to improve the quality of a food (e.g., an animal feed), to improve an animal's performance and/or health, and/or to enhance digestibility of a food or materials within a food.

As used herein, the term “food” is used in a broad sense- and covers food and food products in any form for humans as well as food for animals (i.e. a feed).

The food or feed may be in the form of a solution or as a solid-depending on the use and/or the mode of application and/or the mode of administration. In some embodiments, the enzymes mentioned herein may be used as—or in the preparation or production of—a food or feed substance.

As used herein the term “food or feed ingredient” includes a formulation, which is or can be added to foods or foodstuffs and includes formulations which can be used at low levels in a wide variety of products. The food ingredient may be in the form of a solution or as a solid-depending on the use and/or the mode of application and/or the mode of administration. The enzymes described herein may be used as a food or feed ingredient or in the preparation or production. The enzymes may be—or may not be added to—food supplements. Feed compositions for monogastric animals typically include compositions comprising plant products which contain phytate. Such compositions include, but are not limited to, cornmeal, soybean meal, rapeseed meal, cottonseed meal, maize, wheat, barley and sorghum-based feeds.

As used herein, the term “pelleting” refers to the production of pellets which can be solid, rounded, spherical and cylindrical tablets, particularly feed pellets and solid, extruded animal feed. One example of a known feed pelleting manufacturing process generally includes admixing together food or feed ingredients at least 1 minutes at room temperature, transferring the admixture to a surge bin, conveying the admixture to a steam conditioner (i.e., conditioning), optionally transferring the steam conditioned admixture to an expander, transferring the admixture to the pellet mill or extruder, and finally transferring the pellets into a pellet cooler. (Fairfield, D. 1994. Chapter 10, Pelleting Cost Center. In Feed Manufacturing Technology IV. (McEllhiney, editor), American Feed Industry Association, Arlington, Va., pp. 110-139).

The term “pellet” refers to a composition of animal feed (usually derived from grain) that has been subjected to a heat treatment, such as a steam treatment (i.e., conditioning), and pressed or extruded through a machine. The pellet may incorporate enzyme in the form of a liquid preparation or a dry preparation. The dry preparation may be coated or not coated and may be in the form of a granule. The term “granule” is used for particles composed of enzymes (such as a phytase, for example, any of the engineered phytase polypeptides disclosed herein) and other chemicals such as salts and sugars and may be formed using any of a variety of techniques, including fluid bed granulation approaches to form layered granules.

The term phytase activity in relation to determination in solid or liquid preparations means 1 FTU (phytase unit) which is defined as the amount of enzyme required to release 1 micromole of inorganic orthophosphate from a 5.0 mM Sodium phytate substrate (from rice) in one minute under the reaction conditions, pH 5.5 at 37° C., which are also defined in the ISO 2009 phytase assay-A standard assay for determining phytase activity found at International Standard ISO/DIS 30024:1-17, 2009.

Alternatively, as used herein one unit of phytase (U) can be defined as the quantity of enzyme that releases 1 micromole of inorganic orthophosphate from a 0.2 mM sodium phytate substrate (from rice) in one minute under the reaction conditions 25° C., at pH 5.5 or 3.5 respectively in a Malachite Green assay.

The term “specific activity” as used herein is the number of enzyme units per ml divided by the concentration of (total) protein in mg/ml. Specific activity values are therefore usually quoted as units/mg. Alternatively, specific activity is the number of enzyme units per ml divided by the concentration of phytase in mg/ml.

The term “differential scanning calorimetry” or “DSC” as used herein is a thermoanalytical technique in which the difference in the amount of heat required to increase the temperature of a sample and reference is measured as a function of temperature. Both the sample and reference are maintained at nearly the same temperature throughout the experiment. Generally, the temperature program for a DSC analysis is designed such that the sample holder temperature increases linearly as a function of time. The reference sample should have a well-defined heat capacity over the range of temperatures to be scanned.

The term “prebiotic” means a non-digestible food ingredient that beneficially affects the host by selectively stimulating the growth and/or the activity of one or a limited number of beneficial bacteria.

The term “direct-fed microbial” (“DFM”) as used herein is source of live (viable) microorganisms that when applied in sufficient numbers can confer a benefit to the recipient thereof, i.e., a probiotic. A DFM can comprise one or more of such microorganisms such as bacterial strains. Categories of DFMs include Bacillus, Lactic Acid Bacteria and Yeasts. Thus, the term DFM encompasses one or more of the following: direct fed bacteria, direct fed yeast, direct fed yeast and combinations thereof.

Bacilli are unique, gram-positive rods that form spores. These spores are very stable and can withstand environmental conditions such as heat, moisture and a range of pH. These spores germinate into active vegetative cells when ingested by an animal and can be used in meal and pelleted diets. Lactic Acid Bacteria are gram-positive cocci that produce lactic acid which are antagonistic to pathogens. Since Lactic Acid Bacteria appear to be somewhat heat-sensitive, they are not used in pelleted diets. Types of Lactic Acid Bacteria include Bifidobacterium, Lactobacillus and Streptococcus.

The terms “probiotic,” “probiotic culture,” and “DFM” are used interchangeably herein and define live microorganisms (including bacteria or yeasts for example) which, when for example ingested or locally applied in sufficient numbers, beneficially affects the host organism, i.e. by conferring one or more demonstrable health benefits on the host organism such as a health, digestive, and/or performance benefit. Probiotics may improve the microbial balance in one or more mucosal surfaces. For example, the mucosal surface may be the intestine, the urinary tract, the respiratory tract or the skin. The term “probiotic” as used herein also encompasses live microorganisms that can stimulate the beneficial branches of the immune system and at the same time decrease the inflammatory reactions in a mucosal surface, for example the gut. Whilst there are no lower or upper limits for probiotic intake, it has been suggested that at least 10⁶-10¹², preferably at least 10⁶-10¹⁰, preferably 10⁸-10⁹, cfu as a daily dose will be effective to achieve the beneficial health effects in a subject.

The term “CFU” as used herein means “colony forming units” and is a measure of viable cells in which a colony represents an aggregate of cells derived from a single progenitor cell.

The term “isolated” means a substance in a form or environment that does not occur in nature and does not reflect the extent to which an isolate has been purified but indicates isolation or separation from a native form or native environment. Non-limiting examples of isolated substances include (1) any non-naturally occurring substance, (2) any substance including, but not limited to, any host cell, enzyme, engineered enzyme, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The terms “purify,” “purified,” and purification mean to make substantially pure or clear from unwanted components, material defilement, admixture or imperfection. For example, as applied to nucleic acids or polypeptides, purification generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine(S) is represented as “G087S” or “G87S”. When describing modifications, a position followed by amino acids listed in parentheses indicates a list of substitutions at that position by any of the listed amino acids. For example, 6 (L, I) means position 6 can be substituted with a leucine or isoleucine. At times, in a sequence, a slash (/) is used to define substitutions, e.g. F/V, indicates that the position may have a phenylalanine or valine at that position.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “amino acid” refers to the basic chemical structural unit of a protein or polypeptide. The following abbreviations used herein to identify specific amino acids can be found in Table A.

TABLE A
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The terms “recombinant” and “engineered” refer to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered”, “genetically engineered” and “modified for exogenous gene expression” are used interchangeably herein.

The terms “recombinant construct”, “expression construct”, “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened to obtain lines displaying the desired expression level and pattern. Such screening may be accomplished using standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any plant, organism, or cell of any plant or organism, whether human or non-human into which a recombinant construct can be stably or transiently introduced to express a gene. This term encompasses any progeny of a parent cell, which is not identical to the parent cell due to mutations that occur during propagation.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., cd.) Academic Press (1987); and Sequence Analysis Primer (Gribskov, M. and Devereux, J., eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence. BLAST algorithms refer to the “reference” sequence as the “query” sequence.

As used herein, “homologous proteins” or “homologous phytases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman D J. Gapped BLAST and PSI BLAST a new generation of protein database search programs. Nucleic Acids Res 1997 Set 1; 25 (17): 3389-402). Using this information, proteins sequences can be grouped.

Sequence alignments and percent identity calculations may be performed using the Megalign program of the LASERGENE bioinformatics computing suite (DNASTAR Inc., Madison, WI), the AlignX program of Vector NTI v. 7.0 (Informax, Inc., Bethesda, MD), or the EMBOSS Open Software Suite (EMBL-EBI; Rice et al., Trends in Genetics 16, (6): 276-277 (2000)). Multiple alignment of the sequences can be performed using the CLUSTAL method (such as CLUSTALW; for example, version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13): 3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5. Alternatively, multiple sequence alignment may be derived using MAFFT alignment from Geneious® version 10.2.4 with default settings, scoring matrix BLOSUM62, gap open penalty 1.53 and offset value 0.123.

The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

The term “engineered phytase polypeptide” means that the polypeptide is not naturally occurring and has phytase activity.

It is noted that a fragment of the engineered phytase polypeptide is a portion or subsequence of the engineered phytase polypeptide that is capable of functioning like the engineered phytase polypeptide, i.e., it retains phytase activity.

The term “vector” refers to a polynucleotide sequence designed to introduce nucleic acids into one or more cell types. Vectors include, but are not limited to, cloning vectors, expression vectors, shuttle vectors, plasmids, phage particles, cassettes and the like.

An “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein. “Mature” protein refers to a post-translationally processed polypeptide; i.e., one from which any signal sequence, pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance.

Thus, in one embodiment, there is described a recombinant construct comprising a regulatory sequence functional in a production host operably linked to a nucleotide sequence encoding an engineered phytase polypeptide and fragments thereof as described herein. This recombinant construct may comprise a regulatory sequence functional in a production host operably linked to a nucleotide sequence encoding any of the engineered phytase polypeptide and fragments thereof described herein. Furthermore, the production host is selected from the group consisting of bacteria, fungi, yeast, plants or algae. The preferred production host is the filamentous fungus, Trichoderma reesei.

Alternatively, it may be possible to use cell-free protein synthesis as described in Chong, Curr Protoc Mol Biol. 2014; 108:16.30.1-16.30.11.

Also described herein is a method for producing an engineered phytase polypeptide or fragment thereof comprising:
(a) transforming a production host with the recombinant construct described herein; and
(b) culturing the production host of step (a) under conditions whereby the engineered phytase polypeptide or fragment thereof is produced.

Optionally, the engineered phytase polypeptide or fragment thereof may be recovered from the production host.

In another aspect, a phytase-containing culture supernatant can be obtained by any of the methods disclosed herein.

In another embodiment, there is described a polynucleotide sequence encoding any of the engineered phytase polypeptides or fragments thereof as described herein.

Possible initiation control regions or promoters that can be included in the expression vector are numerous and familiar to those skilled in the art. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art.

In one embodiment, the promoter is one that is native to the host cell. For example, in some instances when Trichoderma reesei is the host, the promoter can be a native T. reesei promoter such as the cbh1 promoter which is deposited in GenBank under Accession Number D86235. Other suitable non-limiting examples of promoters useful for fungal expression include, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63:753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xln1 gene may be useful (see e.g., EPA 137280A1).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The terms “production host”, “production host cell”, “host cell” and “host strains” are used interchangeable herein and mean a suitable host for an expression vector or DNA construct comprising a polynucleotide encoding phytase polypeptide or fragment thereof. The choice of a production host can be selected from the group consisting of bacteria, fungi, yeast, plants and algae. Typically, the choice will depend upon the gene encoding the engineered phytase polypeptide or fragment thereof and its source.

Specifically, host strains are preferably filamentous fungal cells. In a preferred embodiment of the invention, “host cell” means both the cells and protoplasts created from the cells of a filamentous fungal strain and particularly a Trichoderma sp. or an Aspergillus sp.

The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolismis obligatory aerobic. In the present invention, the filamentous fungal parent cell may be a cell of a species of, but not limited to, Trichoderma, (e.g., Trichoderma reesei (previously classified as T. longibrachiatum and currently also known as Hypocrea jecorina), Trichoderma viride, Trichoderma koningii, Trichoderma harzianum); Penicillium sp., Humicola sp. (e.g., Humicola insolens and Humicola grisea); Chrysosporium sp. (e.g., C. lucknowense), Gliocladium sp., Aspergillus sp. (e.g., A. oryzae, A. niger, and A. awamori), Fusarium sp., Neurospora sp., Hypocrea sp., and Emericella sp. (See also, Innis et al., (1985) Sci. 228:21-26).

As used herein, the term “Trichoderma” or “Trichoderma sp.” refer to any fungal genus previously or currently classified as Trichoderma.

An expression cassette can be included in the production host, particularly in the cells of microbial production hosts. The production host cells can be microbial hosts found within the fungal families and which grow over a wide range of temperature, pH values, and solvent tolerances. For example, it is contemplated that any of bacteria, yeast, plants, algae, or fungi such as filamentous fungi, may suitably host the expression vector.

Inclusion of the expression cassette in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

Methods for transforming nucleic acids into filamentous fungi such as Aspergillus spp., e.g., A. oryzae or A. niger, H. grisea, H. insolens, and T. reesei, are well known in the art. A suitable procedure for transformation of Aspergillus host cells is described, for example, in EP238023.

A suitable procedure for transformation of Trichoderma host cells is described, for example, in Steiger et al 2011, Appl. Environ. Microbiol. 77:114-121. Uptake of DNA into the host Trichoderma sp. strain is dependent upon the calcium ion concentration. Generally, between about 10 mM CaCl₂) and 50 mM CaCl₂) is used in an uptake solution. Besides the need for the calcium ion in the uptake solution, other compounds generally included are a buffering system such as TE buffer (10 Mm Tris, pH 7.4; 1 mM EDTA) or 10 mM MOPS, pH 6.0 buffer (morpholinepropanesulfonic acid) and polyethylene glycol (PEG). It is believed that the polyethylene glycol acts to fuse the cell membranes, thus permitting the contents of the medium to be delivered into the cytoplasm of the Trichoderma sp. strain and the plasmid DNA is transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 10⁵ to 10⁷/mL, preferably 2×10⁶/mL are used in transformation. A volume of 100 μL of these protoplasts or cells in an appropriate solution (e.g., 1.2 M sorbitol; 50 mM CaCl₂)) are mixed with the desired DNA. Generally, a high concentration of PEG is added to the uptake solution. From 0.1 to 1 volume of 25% PEG 4000 can be added to the protoplast suspension. However, it is preferable to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells. (see, e.g., U.S. Pat. Nos. 6,022,725 and 6,268,328, both of which are incorporated by reference).

Preferably, genetically stable transformants are constructed with vector systems whereby the nucleic acid encoding the phytase polypeptide or fragment thereof is stably integrated into a host strain chromosome. Transformants are then purified by known techniques.

After the expression vector is introduced into the cells, the transfected or transformed cells are cultured under conditions favoring expression of genes under control of the promoter sequences.

Generally, cells are cultured in a standard medium containing physiological salts and nutrients (see, e.g., Pourquie, J. et al., BIOCHEMISTRY AND GENETICS OF CELLULOSE DEGRADATION, eds. Aubert, J. P. et al., Academic Press, pp. 71-86, 1988 and IImen, M. et al., (1997) Appl. Environ. Microbiol. 63:1298-1306). Common commercially prepared media (e.g., Yeast Malt Extract (YM) broth, Luria Bertani (LB) broth and Sabouraud Dextrose (SD) broth also find use in the present invention.

Culture-conditions are also standard, (e.g., cultures are incubated at approximately 28° C. in appropriate medium in shake cultures or fermenters until desired levels of phytase expression are achieved). Preferred culture conditions for a given filamentous fungus are known in the art and may be found in the scientific literature and/or from the source of the fungi such as the American Type Culture Collection and Fungal Genetics Stock Center.

After fungal growth has been established, the cells are exposed to conditions effective to cause or permit the expression of a phytase and particularly a phytase as defined herein. In cases where a phytase coding sequence is under the control of an inducible promoter, the inducing agent (e.g., a sugar, metal salt or antimicrobial), is added to the medium at a concentration effective to induce phytase expression. An engineered phytase polypeptide or fragment thereof secreted from the host cells can be used, with minimal post-production processing, as a whole broth preparation.

The preparation of a spent whole fermentation broth of a recombinant microorganism can be achieved using any cultivation method known in the art resulting in the expression of an engineered phytase polypeptide or fragment thereof.

The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

After fermentation, a fermentation broth is obtained, the microbial cells and various suspended solids, including residual raw fermentation materials, are removed by conventional separation techniques in order to obtain a phytase solution. Filtration, centrifugation, microfiltration, rotary vacuum drum filtration, ultrafiltration, centrifugation followed by ultrafiltration, extraction, or chromatography, or the like, are generally used.

It is possible to optionally recover the desired protein from the production host. In another aspect, an engineered phytase polypeptide or fragment thereof containing culture supernatant is obtained by using any of the methods known to those skilled in the art.

Examples of these techniques include, but are not limited to, affinity chromatography (Tilbeurgh et a., (1984) FEBS Lett. 16:215), ion-exchange chromatographic methods (Goyal et al., (1991) Biores. Technol. 36:37; Fliess et al., (1983) Eur. J. Appl. Microbiol. Biotechnol. 17:314; Bhikhabhai et al, (1984) J. Appl. Biochem. 6:336; and Ellouz et al., (1987) Chromatography 396:307), including ion-exchange using materials with high resolution power (Medve et al., (1998) J. Chromatography A 808:153), hydrophobic interaction chromatography (See, Tomaz and Queiroz, (1999) J. Chromatography A 865:123; two-phase partitioning (See, Brumbauer, et al., (1999) Bioseparation 7:287); ethanol precipitation; reverse phase HPLC, chromatography on silica or on a cation-exchange resin such as DEAE, chromatofocusing, SDS-PAGE, ammonium sulfate precipitation, and gel filtration (e.g., Sephadex G-75). The degree of purification desired will vary depending on the use of the engineered phytase polypeptide or fragment thereof. In some embodiments, purification will not be necessary. On the other hand, it may be desirable to concentrate a solution containing an engineered phytase polypeptide or fragment thereof in order to optimize recovery. Use of unconcentrated solutions requires increased incubation time in order to collect the enriched or purified enzyme precipitate. The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

In addition, concentration of the desired protein product may be performed using, e.g., a precipitation agent, such as a metal halide precipitation agent. The metal halide precipitation agent, sodium chloride, can also be used as a preservative. The metal halide precipitation agent is used in an amount effective to precipitate the engineered phytase polypeptide or fragment thereof. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing. Generally, at least about 5% w/v (weight/volume) to about 25% w/v of metal halide is added to the concentrated enzyme solution, and usually at least 8% w/v.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously. Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. Additional organic compounds also include but are not limited to 4-hydroxybenzoic acid methyl ester (named methyl PARABEN), 4-hydroxybenzoic acid propyl ester (named propyl PARABEN). For further descriptions, see, e.g., U.S. Pat. No. 5,281,526. Addition of the organic compound precipitation agent provides the advantage of high flexibility of the precipitation conditions with respect to pH, temperature, concentration, precipitation agent, protein concentration, and time of incubation. Generally, at least about 0.01% w/v and no more than about 0.3% w/v of organic compound precipitation agent is added to the concentrated enzyme solution.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

Sometimes it is advantageous to delete genes from expression hosts, where the gene deficiency can be cured by an expression vector. Where it is desired to obtain a fungal host cell having one or more inactivated genes known methods may be used (e.g. methods disclosed in U.S. Pat. Nos. 5,246,853, 5,475,101 and WO92/06209). Gene inactivation may be accomplished by complete or partial deletion, by insertional inactivation or by any other means which renders a gene nonfunctional for its intended purpose (such that the gene is prevented from expression of a functional protein).

Any gene from a Trichoderma sp or other filamentous fungal host, which has been cloned can be deleted, for example cbh1, cbh2, egl1 and egl2 genes. In some embodiments, gene deletion may be accomplished by inserting a form of the desired gene to be inactivated into a plasmid by methods known in the art. The deletion plasmid is then cut at an appropriate restriction enzyme site(s), internal to the desired gene coding region, and the gene coding sequence or part thereof is replaced with a selectable marker. Flanking DNA sequences from the locus of the gene to be deleted (preferably between about 0.5 to 2.0 kb) remain on either side of the marker gene. An appropriate deletion plasmid will generally have unique restriction enzyme sites present therein to enable the fragment containing the deleted gene, including the flanking DNA sequences and the selectable markers gene to be removed as a single linear piece.

Depending upon the host cell used post-transcriptional and/or post-translational modifications may be made. One non-limiting example of a post-transcriptional and/or post-translational modification is “clipping” or “truncation” of a polypeptide. In another instance, this clipping may result in taking a mature phytase polypeptide and further removing N or C-terminal amino acids to generate truncated forms of the phytase that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

Further sequence modifications of polypeptides post expression may occur. This includes, but is not limited to, oxidation, deglycosylation, glycation, etc. It is known that glycation can affect the activity of phytase when subjected to incubation with glucose or other reducing sugars especially at temperatures above 30° C. and neutral or alkaline pH. Protein engineering to eliminate Lysine residues can be used to prevent such modification. An example of this can be found in U.S. Pat. No. 8,507,240. For example, yeast expression can result in highly glycosylated polypeptides resulting in an apparent increased molecular weight. Also, WO2013/119470 (incorporated by reference herein) having international publication date Aug. 15, 2013 relates to phytases having increased stability believed to be due to increased glycosylation.

The term “glycosylation” as used herein refers to the attachment of glycans to molecules, for example to proteins. Glycosylation may be an enzymatic reaction. The attachment formed may be through covalent bonds. The phrase “highly glycosylated” refers to a molecule such as an enzyme which is glycosylated in many sites and at all or nearly all the available glycosylation sites, for instance N-linked glycosylation sites. Alternatively, or in addition to, the phrase “highly glycosylated” can refer to extensive glycolytic branching (such as, the size and number of glycolytic moieties associated with a particular N-linked glycosylation site) at all or substantially all N-linked glycosylation sites. In some embodiments, the engineered phytase polypeptide is glycosylated at all or substantially all consensus N-linked glycosylation sites (i.e. an NXS/T consensus N-linked glycosylation site).

The term “glycan” as used herein refers to a polysaccharide or oligosaccharide, or the carbohydrate section of a glycoconjugate such as a glycoprotein. Glycans may be homo- or heteropolymers of monosaccharide residues. They may be linear or branched molecules.

A phytase may have varying degrees of glycosylation. It is known that such glycosylations may improve stability during storage and in applications. Extensive

The activity of any of the engineered phytase polypeptides or fragments thereof disclosed herein can be determined as discussed above.

It is believed that applying a robust engineered phytase polypeptide or fragment thereof to feed in a liquid form is beneficial as compared to applying such a phytase as a coated granule. This coated granule is the current commercial approach to make phytase products suitable for high temperature conditioning and pelleting. Benefits of liquid application of robust enzyme include; 1) the enzyme will start to work immediately after ingestion by an animal since it does not have to be released from the coated granule before it can interact with the feed, 2) there is improved distribution of the enzyme throughout the feed, thus, ensuring a more consistent delivery of the enzyme to the animal which is particularly important for young animals that eat small amounts of feed, 3) even distribution in the feed makes it easier to measure the enzyme in the feed, and 4) in the case of a robust phytase, such as the engineered phytase polypeptide and fragment disclosed herein, it may start to degrade phytate already present in the feed.

In other words, the novel engineered phytase polypeptides and fragments thereof are so robust that no special coating or formulation is believed to be needed to apply them to feed prior to conditioning and pelleting since they have been engineered to withstand the stress of conditioning and pelleting used in industrial feed production. Accordingly, the robustness of the novel engineered phytase polypeptides and fragments thereof described herein is such that they can be applied as an uncoated granule or particle or uncoated and unprotected when put into a liquid.

It should be noted that the engineered phytase polypeptides and fragments thereof can be formulated inexpensively on a solid carrier without specific need for protective coatings and still maintain activity throughout the conditioning and pelleting process. A protective coating to provide additional thermostability when applied in a solid form can be beneficial for obtaining pelleting stability when required in certain regions where harsher conditions are used or if conditions warrant it, e.g., as in the case of super conditioning feed above 90° C.

The disclosed engineered phytase polypeptides or fragments thereof were derived using a combination of methods and techniques know in the field of protein engineering which include, phylogenetic analysis, site evaluation libraries, combinatorial libraries, high throughput screening and statistical analysis.

In one aspect, the disclosure relates to an engineered phytase polypeptide or fragment thereof also that has at least 82% sequence identity with the amino acid sequence of SEQ ID NO: 1.

Those skilled in the art will appreciate that such at least 82% sequence identity also includes 82%, 83%, 84%, 85%, 86%, 87%, 88%, 89%, 90%, 91%, 92%, 93%, 94%, 95%, 96%, 97%, 98%, 99% or 100%.

Those skilled in the art will appreciate that at least 79% sequence identity also includes 79%, 80%, 81%, 82%, 83%, 84%, 85%, 86%, 87%, 88%, 89%, 90%, 91%, 92%, 93%, 94%, 95%, 96%, 97%, 98%, 99% or 100%.

There can also be mentioned the following in that in some embodiments, there is provided:
a) an engineered phytase polypeptide or fragment thereof also that has at least 81% (such as 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 2, 3, 8, 10, 12, 18, 19, 24, 26, 27, 28, 30, 31, 32, 33, and/or 36.
b) an engineered phytase polypeptide or fragment thereof also that has at least 82% (such as 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 1, 4, 5, 7, 9, 11, 14, 15, 17, 21, 25, 34, and/or 35;
c) an engineered phytase polypeptide or fragment thereof also that has at least 83% (such as, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NO:13;
d) an engineered phytase polypeptide or fragment thereof also that has at least 79% (such as, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 6, and/or 22; and/or
e) an engineered phytase polypeptide or fragment thereof also that has at least 80% (such as, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 16, 20, 23, 29, and/or 37.

In further aspects, the polypeptide comprises a core domain of an engineered phytase polypeptide or is a core domain fragment of an engineered phytase polypeptide. A “core domain fragment” is herein defined as a polypeptide having one or more amino acids deleted from the amino and/or carboxyl terminus of the polypeptide. As used herein, the phrase “core domain” refers to a polypeptide region encompassing amino acids necessary to maintain the structure and function (such as, phytic acid hydrolysis) of the polypeptide. Amino acids in the core domain can be further modified to improve thermostability or catalytic activity under various conditions such as, without limitation, pH. In some non-limiting embodiments, the core domain of the engineered phytase polypeptides or fragment thereof disclosed herein corresponds to amino acid positions 14-325 of SEQ ID NO:1. In other non-limiting embodiments, the core domain corresponds to amino acid positions 13-326, 12-327, 11-328, 10-329, 9-330, 8-331, 7-332, 6-333, 5-334, 4-335, 3-336, 2-337, or 1-338 of SEQ ID NO:1. In other embodiments, the N-terminus of the core domain corresponds to amino acid position 1, 2, 3, 4, 5, 6, 7, 8, 9, 10, 11, 12, 13, 14, 15, 16, 17, 18, 19, 20, 21, 22, 23, 24, or 25 of SEQ ID NO: 1 and the C-terminus of the core domain corresponds to amino acid position 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, or 413 of SEQ ID NO:1.

Accordingly, also provided herein are:
f) an engineered phytase polypeptide or core domain fragment thereof that has at least 78% (such as, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NO:6, wherein said amino acid positions correspond to those of SEQ ID NO:1;
g) an engineered phytase polypeptide or core domain fragment thereof that has at least 79% (such as, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 2, 8, 27, and/or 37, wherein said amino acid positions correspond to those of SEQ ID NO: 1;
h) an engineered phytase polypeptide or core domain fragment thereof that has at least 81% (such as, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 3, 10, 12, 18, 25, 26, 28, 30, 32, and/or 35, wherein said amino acid positions correspond to those of SEQ ID NO:1;
i) an engineered phytase polypeptide or core domain fragment thereof that has at least 82% (such as, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 1, 4, 5, 7, 9, 11, 13-17, 21, 22, 31, 33, 34, and/or 36, wherein said amino acid positions correspond to those of SEQ ID NO:1;
j) an engineered phytase polypeptide or core domain fragment thereof that has at least 83% (such as, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 19, 20, 23, and/or 24, wherein said amino acid positions correspond to those of SEQ ID NO:1; and/or
k) an engineered phytase polypeptide or core domain fragment thereof that has at least 84% (such as, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NO:29, wherein said amino acid positions correspond to those of SEQ ID NO:1.

In another aspect, any of the engineered polypeptides or fragments thereof disclosed herein comprise a specific activity of at least about 100 U/mg at pH 3.5. The specific activity range (U/mg at pH 3.5) includes, but is not limited to, about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 2000, etc.

In another aspect, some of the engineered polypeptides or fragments thereof disclosed herein comprise a specific activity of at least about 100 U/mg at pH 5.5. The specific activity range (U/mg at pH 5.5) includes, but is not limited to, about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 2000, etc.

In still another aspect, any of the engineered phytase polypeptides or fragments thereof disclosed herein may be stable in a liquid form at a pH about 3.0 or lower. This is very relevant when engineered phytase polypeptides or fragments thereof described herein are passing through the digestive tract of an animal as is discussed below.

In another embodiment, there is described non-trace mineral-containing diet comprising any of the engineered phytase polypeptides or fragments thereof described herein.

Importantly, feed additive enzymes e.g. a phytase is subjected to very harsh conditions as it passes through the digestive track of an animal, i.e. low pH and presence of digestive enzymes. Pepsin is one of the most important proteolytic digestive enzymes present in the gastrointestinal tract of monogastric animals. Pepsin has low specificity and high pH tolerance in the acidic area (pH 1.5-6.0 stabile up to pH 8.0). The engineered phytase polypeptides or fragments thereof described herein are largely resistant against pepsin, which is necessary for good in-vivo performance.

The non-trace mineral-containing diets comprising any of the engineered phytase polypeptides or fragments thereof described herein may be used (i) alone or (ii) in combination with a direct fed microbial comprising at least one bacterial strain or (iii) with at least one other enzyme or (iv) in combination with a direct fed microbial comprising at least one bacterial strain and at least one other enzyme, or (v) any of (i), (ii), (iii) or (iv) further comprising at least one other feed additive component and, optionally, the engineered phytase polypeptide or fragment thereof is present in an amount of at least 0.1 g/ton feed (such as at least about 0.1 g/ton, 0.2 g/ton, 0.3 g/ton, 0.4 g/ton, 0.5 g/ton, 0.6 g/ton, 0.7 g/ton, 0.8 g/ton, 0.9 g/ton, 1 g/ton, 1.1 g/ton, 1.2 g/ton, 1.3 g/ton, 1.4 g/ton, 1.5 g/ton, 1.6 g/ton, 1.7 g/ton, 1.8 g/ton, 1.9 g/ton, 2 g/ton, 2.1 g/ton, 2.2 g/ton, 2.3 g/ton, 2.4 g/ton, 2.5 g/ton, 2.6 g/ton, 2.7 g/ton, 2.8 g/ton, 2.9 g/ton, 3 g/ton, or more).

In some non-limiting embodiments, the phytase is present in the diet in range of about 200 FTU/kg to about 1000 FTU/kg fccd, more preferably about 300 FTU/kg feed to about 750 FTU/kg feed, more preferably about 400 FTU/kg feed to about 500 FTU/kg feed. In one embodiment, the phytase is present in the feedstuff at more than about 200 FTU/kg feed, suitably more than about 300 FTU/kg feed, suitably more than about 400 FTU/kg feed. In one embodiment, the phytase is present in the feedstuff at less than about 1000 FTU/kg feed, suitably less than about 750 FTU/kg feed. I some embodiments, the phytase is present in the feed additive composition in range of about 40 FTU/g to about 40,000 FTU/g composition, more preferably about 80 FTU/g composition to about 20,000 FTU/g composition, and even more preferably about 100 FTU/g composition to about 10,000 FTU/g composition, and even more preferably about 200 FTU/g composition to about 10,000 FTU/g composition. In one embodiment, the phytase is present in the feed additive composition at more than about 40 FTU/g composition, suitably more than about 60 FTU/g composition, suitably more than about 100 FTU/g composition, suitably more than about 150 FTU/g composition, suitably more than about 200 FTU/g composition. In one embodiment, the phytase is present in the feed additive composition at less than about 40,000 FTU/g composition, suitably less than about 20,000 FTU/g composition, suitably less than about 15,000 FTU/g composition, suitably less than about 10,000 FTU/g composition.

In some non-limiting embodiments, “1 FTU” (phytase unit) is defined as the amount of enzyme required to release 1 μmol of inorganic orthophosphate from a substrate in one minute under the reaction conditions defined in the ISO 2009 phytase assay-A standard assay for determining phytase activity and 1 FTU can be found at International Standard ISO/DIS 30024:1-17, 2009. In one embodiment, the enzyme is classified using the E.C. classification above, and the E.C. classification designates an enzyme having that activity when tested in the assay taught herein for determining 1 FTU.

The terms “feed additive”, “feed additive components”, and/or “feed additive ingredients” are used interchangeably herein.

Feed additives can be described as products used in animal nutrition for purposes of improving the quality of feed and the quality of food from animal origin, or to improve the animals' performance and health, e.g. providing enhanced digestibility of the feed materials.

Feed additives fall into a number of categories such as sensory additives which stimulate an animal's appetite so that they naturally want to cat more. Nutritional additives provide a particular nutrient that may be deficient in an animal's diet. Zootechnical additives improve the overall nutritional value of an animal's diet through additives in the feed.

As used herein, a “non-trace mineral-containing diet” refers to a diet that contains no to substantially no trace minerals exogenously added, for example, as a feed additive.

Examples of such feed additives include, but are not limited to, prebiotics, essential oils (such as, without limitation, thymol and/or cinnamaldehyde), fatty acids, short chain fatty acids such as propionic acid and butyric acid, etc., vitamins, minerals, amino acids, etc.

Feed additive compositions or formulations may also comprise at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

At least one other enzyme (i.e. in addition to any of the engineered phytase polypeptides or fragments thereof disclosed herein) can be included in the feed additive compositions or formulations disclosed herein which can include, but are not limited to, a xylanase, amylase, another phytase, beta-glucanase, and/or a protease.

Xylanase is the name given to a class of enzymes that degrade the linear polysaccharide β-1,4-xylan into xylose, thus breaking down hemicellulose, one of the major components of plant cell walls. Xylanases, e.g., endo-β-xylanases (EC 3.2.1.8) hydrolyze the xylan backbone chain.

In one embodiment, the xylanase may be any commercially available xylanase. Suitably the xylanase may be an endo-1,4-P-d-xylanase (classified as E.G. 3.2.1.8) or a 1,4β-xylosidase (classified as E.G. 3.2.1.37). In one embodiment, the disclosure relates to a composition comprising any of the engineered phytase polypeptides or fragments thereof disclosed herein in combination with an endoxylanase, e.g. an endo-1,4-P-d-xylanase, and another enzyme. All E.C. enzyme classifications referred to herein relate to the classifications provided in Enzyme Nomenclature—Recommendations (1992) of the nomenclature committee of the International Union of Biochemistry and Molecular Biology—ISBN 0-12-226164-3, which is incorporated herein.

In another embodiment, the xylanase may be a xylanase from Bacillus, Trichodermna, Therinomyces, Aspergillus, Humicola and Penicillium. In still another embodiment, the xylanase may be the xylanase in Axtra XAP® or Avizyme 1502®, both commercially available products from Danisco A/S. In one embodiment, the xylanase may be a mixture of two or more xylanases. In still another embodiment, the xylanase is an endo-1,4-β-xylanase or a 1,4-β-xylosidase.

In one embodiment, the disclosure relates to a non-trace mineral-containing diet comprising any of the engineered phytase polypeptides or fragments thereof disclosed herein and a xylanase. In one embodiment, the non-trace minerals-containing diet comprises 10-50, 50-100, 100-150, 150-200, 200-250, 250-300, 300-350, 350-400, 400-450, 450-500, 500-550, 550-600, 600-650, 650-700, 700-750, and greater than 750 xylanase units/g of composition.

In one embodiment, the non-trace mineral-containing diet comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, and greater than 8000 xylanase units/g composition.

It will be understood that one xylanase unit (XU) is the amount of enzyme that releases 0.5 μmol of reducing sugar equivalents (as xylose by the Dinitrosalicylic acid (DNS) assay-reducing sugar method) from an oat-spelt-xylan substrate per min at pH 5.3 and 50° C. (Bailey, et al., Journal of Biotechnology, Volume 23, (3), May 1992, 257-270).

Amylase is a class of enzymes capable of hydrolysing starch to shorter-chain oligosaccharides, such as maltose. The glucose moiety can then be more easily transferred from maltose to a monoglyceride or glycosylmonoglyceride than from the original starch molecule. The term amylase includes α-amylases (E.G. 3.2.1.1), G4-forming amylases (E.G. 3.2.1.60), β-amylases (E.G. 3.2.1.2) and γ-amylases (E.C. 3.2.1.3). Amylases may be of bacterial or fungal origin, or chemically modified or protein engineered mutants.

In one embodiment, the amylase may be a mixture of two or more amylases. In another embodiment, the amylase may be an amylase, e.g. an α-amylase, from Bacillus licheniformis and an amylase, e.g. an α-amylase, from Bacillus amyloliquefaciens. In one embodiment, the α-amylase may be the α-amylase in Axtra XAP® or Avizyme 1502®, both commercially available products from Danisco A/S. In yet another embodiment, the amylase may be a pepsin resistant α-amylase, such as a pepsin resistant Trichoderma (such as Trichoderma reesei) alpha amylase. A suitably pepsin resistant α-amylase is taught in UK application number 101 1513.7 (which is incorporated herein by reference) and PCT/IB2011/053018 (which is incorporated herein by reference).

It will be understood that one amylase unit (AU) is the amount of enzyme that releases 1 mmol of glucosidic linkages from a water insoluble cross-linked starch polymer substrate per min at pH 6.5 and 37° C. (this may be referred to herein as the assay for determining 1 AU).

In one embodiment, disclosure relates to a non-trace mineral-containing diet comprising any of the engineered phytase polypeptides or fragments thereof disclosed herein and an amylase. In one embodiment, disclosure relates to a non-trace mineral-containing diet comprising any of the engineered phytase polypeptides or fragments thereof disclosed herein, xylanase and amylase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 amylase units/g composition.

In one embodiment, the non-trace mineral-containing diet comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 amylase units/g composition.

The term protease as used herein is synonymous with peptidase or proteinase. The protease may be a subtilisin (E.G. 3.4.21.62) or a bacillolysin (E.G. 3.4.24.28) or an alkaline serine protease (E.G. 3.4.21.x) or a keratinase (E.G. 3.4.X.X). In one embodiment, the protease is a subtilisin. Suitable proteases include those of animal, vegetable or microbial origin.

Chemically modified or protein engineered mutants are also suitable. The protease may be a serine protease or a metalloprotease. e.g., an alkaline microbial protease or a trypsin-like protease. In one embodiment, provided herein are compositions comprising any of the engineered phytase polypeptides or fragments thereof disclosed herein and one or more protease.

Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115.

In one embodiment, the protease is selected from the group consisting of subtilisin, a bacillolysin, an alkine serine protease, a keratinase, and a Nocardiopsis protease.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40° C. This may be referred to as the assay for determining 1 PU.

In one embodiment, disclosure relates to a non-trace mineral-containing diet comprising any of the engineered phytase polypeptides or fragments thereof disclosed herein and a protease. In another embodiment, disclosure relates to a non-trace mineral-containing diet comprising any of the engineered phytase polypeptides or fragments thereof disclosed herein and a xylanase and a protease. In still another embodiment, the disclosure relates to a non-trace mineral-containing diet comprising any of the engineered phytase polypeptides or fragments thereof disclosed herein and an amylase and a protease. In yet another embodiment, the disclosure relates to a non-trace mineral-containing diet comprising any of the engineered phytase polypeptides or fragments thereof disclosed herein and a xylanase, an amylase and a protease.

In one embodiment, the non-trace mineral-containing diet comprises about 10-50, 50-100, 100-150, 150-200, 200-250, 250-300, 300-350, 350-400, 400-450, 450-500, 500-550, 550-600, 600-650, 650-700, 700-750, and greater than 750 protease units/g composition.

In one embodiment, the non-trace mineral-containing diet comprises about 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 protease units/g composition.

In other embodiments, the diet can have reduced (such as substantially reduced) trace mineral levels relative to those recommended by the National Research Council (NRC) or broiler breeders. In some embodiments, the diets contain from between 0.05% to about 75% trace mineral levels relative to those recommended by the National Research Council (NRC) or broiler breeders, such as any of about 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% trace mineral levels relative to those recommended by the National Research Council (NRC) or broiler breeders (such as the recommended levels discussed in the Examples section below). In some embodiments, the trace minerals are one or more of zinc (Zn), iron (Fe), copper (Cu), manganese (Mn) and/or selenium (Se).

At least one direct fed microbial (DFM) may comprise at least one viable microorganism such as a viable bacterial strain or a viable yeast or a viable fungi. Preferably, the DFM comprises at least one viable bacteria.

It is possible that the DFM may be a spore forming bacterial strain and hence the term DFM may be comprised of or contain spores, e.g. bacterial spores. Thus, the term “viable microorganism” as used herein may include microbial spores, such as endospores or conidia. Alternatively, the DFM in the feed additive composition described herein may not comprise of or may not contain microbial spores, e.g. endospores or conidia.

The microorganism may be a naturally-occurring microorganism or it may be a transformed microorganism.

A DFM as described herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera and combinations thereof.

Preferably, the DFM comprises one or more bacterial strains selected from the following Bacillus spp: Bacillus subtilis, Bacillus cereus, Bacillus licheniformis, Bacillus pumilis and Bacillus amyloliquefaciens.

The genus “Bacillus”, as used herein, includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilis and B. thuringiensis. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In another aspect, the DFM may be further combined with the following Lactococcus spp: Lactococcus cremoris and Lactococcus lactis and combinations thereof.

The DFM may be further combined with the following Lactobacillus spp: Lactobacillus buchneri, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii and Lactobacillus jensenii, and combinations of any thereof.

In still another aspect, the DFM may be further combined with the following Bifidobacteria spp: Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

There can be mentioned bacteria of the following species: Bacillus subtilis, Bacillus licheniformis, Bacillus amyloliquefaciens, Bacillus pumilis, Enterococcus, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Bacillus subtilis, Propionibacterium thoenii, Lactobacillus farciminis, Lactobacillus rhamnosus, Megasphaera elsdenii, Clostridium butyricum, Bifidobacterium animalis ssp. animalis, Lactobacillus reuteri, Bacillus cereus, Lactobacillus salivarius ssp. Salivarius, Propionibacteria sp and combinations thereof.

A direct-fed microbial described herein comprising one or more bacterial strains may be of the same type (genus, species and strain) or may comprise a mixture of genera, species and/or strains.

Alternatively, a DFM may be combined with one or more of the products or the microorganisms contained in those products disclosed in WO2012110778, and summarized as follows: Bacillus strain 2084 Accession No. NRRLB-50013, Bacillus strain LSSAO1 Accession No. NRRL B-50104, and Bacillus strain 15A-P4 ATCC Accession No. PTA-6507 (from Enviva Pro®. (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro®Tect®); Enterococcus and Pediococcus (from Poultry Star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Lactobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactis DSM 1103 (from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilacti (from Bactocell); Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from InteSwine®); Saccharomyces cerevisia (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM I-1012 (from TOYOCERIN®), or other DFMs such as Bacillus licheniformis and Bacillus subtilis (from BioPlus® YC) and Bacillus subtilis (from GalliPro®).

The DFM may be combined with Enviva® PRO which is commercially available from Danisco A/S. Enviva Pro® is a combination of Bacillus strain 2084 Accession No. NRRL B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference).

It is also possible to combine the DFM described herein with a yeast from the genera: Saccharomyces spp.

Preferably, the DFM described herein comprises microorganisms which are generally recognized as safe (GRAS) and, preferably are GRAS-approved.

A person of ordinary skill in the art will readily be aware of specific species and/or strains of microorganisms from within the genera described herein which are used in the food and/or agricultural industries and which are generally considered suitable for animal consumption.

In some embodiments, it is important that the DFM be heat tolerant, i.e. is thermotolerant. This is particularly the case when the feed is pelleted. Therefore, in another embodiment, the DFM may be a thermotolerant microorganism, such as a thermotolerant bacteria, including for example Bacillus spp.

In other aspects, it may be desirable that the DFM comprises a spore producing bacteria, such as Bacilli, e.g. Bacillus spp. Bacilli are able to form stable endospores when conditions for growth are unfavorable and are very resistant to heat, pH, moisture and disinfectants.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effect) of a pathogen.

As described above, the DFM may be any suitable DFM. For example, the following assay “DFM ASSAY” may be used to determine the suitability of a microorganism to be a DFM. The DFM assay as used herein is explained in more detail in US2009/0280090. For avoidance of doubt, the DFM selected as an inhibitory strain (or an antipathogenic DFM) in accordance with the “DFM ASSAY” taught herein is a suitable DFM for use in accordance with the present disclosure, i.e. in the feed additive composition according to the present disclosure.

Tubes were seeded each with a representative pathogen (e.g., bacteria) from a representative cluster.

Supernatant from a potential DFM, grown aerobically or anaerobically, is added to the seeded tubes (except for the control to which no supernatant is added) and incubated. After incubation, the optical density (OD) of the control and supernatant treated tubes was measured for each pathogen.

Colonies of (potential DFM) strains that produced a lowered OD compared with the control (which did not contain any supernatant) can then be classified as an inhibitory strain (or an antipathogenic DFM). Thus, The DFM assay as used herein is explained in more detail in US2009/0280090.

Preferably, a representative pathogen used in this DFM assay can be one (or more) of the following: Clostridium, such as Clostridium perfringens and/or Clostridium difficile, and/or E. coli and/or Salmonella spp and/or Campylobacter spp. In one preferred embodiment, the assay is conducted with one or more of Clostridium perfringens and/or Clostridium difficile and/or E. coli, preferably Clostridium perfringens and/or Clostridium difficile, more preferably Clostridium perfringens.

Antipathogenic DFMs include one or more of the following bacteria and are described in WO2013029013:
Bacillus subtilis strain 3BP5 Accession No. NRRL B-50510,
Bacillus strain 918 ATCC Accession No. NRRL B-50508, and
Bacillus strain 1013 ATCC Accession No. NRRL B-50509.

DFMs may be prepared as culture(s) and carrier(s) (where used) and can be added to a ribbon or paddle mixer and mixed for about 15 minutes, although the timing can be increased or decreased. The components are blended such that a uniform mixture of the cultures and carriers result. The final product is preferably a dry, flowable powder. The DFM(s) comprising one or more bacterial strains can then be added to animal feed or a feed premix, added to an animal's water, or administered in other ways known in the art (preferably simultaneously with the enzymes described herein.

Inclusion of the individual strains in the DFM mixture can be in proportions varying from 1% to 99% and, preferably, from 25% to 75%

Suitable dosages of the DFM in animal feed may range from about 1×10³ CFU/g feed to about 1×10¹⁰ CFU/g feed, suitably between about 1×10⁴ CFU/g feed to about 1×10⁸ CFU/g feed, suitably between about 7.5×10⁴ CFU/g feed to about 1×10⁷ CFU/g feed.

In another aspect, the DFM may be dosed in feedstuff at more than about 1×10³ CFU/g feed, suitably more than about 1×10⁴ CFU/g feed, suitably more than about 5×10⁴ CFU/g feed, or suitably more than about 1×10⁵ CFU/g feed.

The DFM may be dosed in a non-trace mineral-containing diet from about 1×10³ CFU/g composition to about 1×10¹³ CFU/g composition, preferably 1×10⁵ CFU/g composition to about 1×10¹³ CFU/g composition, more preferably between about 1×10⁶ CFU/g composition to about 1×10¹² CFU/g composition, and most preferably between about 3.75×10⁷ CFU/g composition to about 1×10¹¹ CFU/g composition. In another aspect, the DFM may be dosed in a feed additive composition at more than about 1×10⁵ CFU/g composition, preferably more than about 1×10⁶ CFU/g composition, and most preferably more than about 3.75×10⁷ CFU/g composition. In one embodiment, the DFM is dosed in the feed additive composition at more than about 2×10⁵ CFU/g composition, suitably more than about 2×10⁶ CFU/g composition, suitably more than about 3.75×10⁷ CFU/g composition.

In some aspects, at least one polypeptide having phytase activity as described herein can itself (or in other embodiments, in combination with one or more DFMs, such as any of those disclosed herein) reduce the levels of pathogenic bacterial in animals. Fresh food animal products, including poultry, are susceptible to contamination by microorganisms that contact meat surfaces immediately after slaughter and evisceration, including organisms in the gastrointestinal tracts which can be transferred during processing. Contaminating microorganisms include bacteria such as Salmonella and Campylobacter species, Listeria monocytogenes, Escherichia coli and other coliforms, and other enteric organisms. Once pathogenic bacteria contact tissue surfaces, they rapidly attach and are difficult to remove even with chlorine disinfectant permitted for use in poultry sprays and chill tanks.

The problems created by Salmonella bacteria in poultry products are particularly noteworthy. Currently, Americans spend approximately $20 billion annually on poultry products, consuming about 80 pounds per capita. Approximately 35% to 45% of poultry reaching U.S. consumers is contaminated with Salmonella species. Improper cooking and physical transfer of the bacteria to food handling surfaces and thereafter to other foods result in the spread of the microorganisms, causing gastrointestinal disorders and, in some cases, death.

Additionally, Campylobacter is a major cause of human foodborne gastroenteritis globally, particularly in Europe, and poultry reservoirs have been associated with over 80% of human cases. Campylobacter can cause an illness called campylobacteriosis. With over 246,000 human cases annually, it is the most frequently reported foodborne illness in the EU. The cost of campylobacteriosis to public health systems and to lost productivity in the EU is estimated by EFSA to be around €2.4 billion a year (worldwideweb.efsa.europa.eu/en/topics/topic/campylobacter).

Despite the importance to human health, there are currently no consistent solutions to controlling Campylobacter. It is widely accepted by animal producers, the food industry, and Regulators that a farm to fork approach is required to help reduce prevalence, contamination levels and the risk of human infection. The poultry intestinal tract is a reservoir for Campylobacter, with approximately 10⁹ CFU/gram caecal content. This reservoir results in cross-contamination of negative flocks and equipment at processing. National surveillance programs for Campylobacter in poultry requiring routine testing for Campylobacter are scarce in markets other than the E.U. and those markets exporting to European countries. For Campylobacter, it is expected that a level of 10⁸ CFU/gram caccal content (10⁶ CFU/gram caccal content stretch goal) is required consistently to reduce neck skin levels post-chilling (the Regulator's sample type).

As such, in some aspects, provided herein is a method for reducing pathogenic bacteria populations in animals comprising administering an effective amount of an animal diet comprising an engineered phytase polypeptide or a fragment thereof comprising phytase activity (such as any of those disclosed herein); and lacking one or more exogenously added trace minerals (such as, without limitation, iron and/or inorganic phosphate) to an animal.

In some embodiments, the pathogenic bacteria is reduced by at least about 1%, 2%, 3%, 4%, 5%, 6%, 7%, 8%, 9%, 105, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, or more (inclusive of percentages falling in-between these values) as compared to a feed which does not comprise said phytase-containing low trace mineral content feed additive compositions or animal feed diets described herein. The pathogenic bacteria can be, without limitation, one or more of Streptococcus pneumoniae, Campylobacter (e.g. C. jejuni), Neisseria gonorrhoeae, Staphylococcus aureus, Shigella, Enterococcus, Staphylococcus aureus, Streptococcus, Enterobacteriaceae, Acinetobacter, Clostridium (e.g., C. difficile or C. perfingens, E. coli (such as, ETEC), Pseudomonas aeruginosa, H. pylori, Streptococcus anginosus and Uropathogenic E. coli (UPEC). In some embodiments the animal is swine. In other embodiments, the animal is poultry.

In still another aspect, there is disclosed a non-trace mineral-containing diet for use in animal feed comprising at least one polypeptide having phytase activity as described herein, used either alone or in combination with at least one direct fed microbial or in combination with at least one other enzyme or in combination with at least one direct fed microbial and at least one other enzyme, wherein the feed additive composition comprises may be in any form such as a granulated particle. Such granulated particles may be produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

Furthermore, particles of the granulated feed additive composition can have a mean diameter of greater than 50 microns and less than 2000 microns.

Those skilled in the art will understand that animal feed may include plant material such as corn, wheat, sorghum, soybean, canola, sunflower or mixtures of any of these plant materials or plant protein sources for poultry, pigs, ruminants, aquaculture and pets. It is contemplated that animal performance parameters, such as growth, feed intake and feed efficiency, but also improved uniformity, reduced ammonia concentration in the animal house and consequently improved welfare and health status of the animals will be improved.

Thus, there is disclosed a method for improving the nutritional value of an animal feed, wherein any of the engineered phytases or fragments thereof as described herein can be added to animal feed.

The phrase, an “effective amount” as used herein, refers to the amount of an active agent (such as, a phytase, e.g. any of the engineered phytase polypeptides disclosed herein) required to confer improved performance on an animal on one or more metrics (such as, without limitation, one or more of increased feed efficiency, increased weight gain, reduced feed conversion ratio, improved digestibility of nutrients or energy in a feed, improved nitrogen retention, improved ability to avoid the negative effects of necrotic enteritis, and improved immune response), either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or by the digestibility of a nutrient in a feed (e.g., amino acid digestibility or phosphorus digestibility) and/or digestible energy or metabolizable energy in a feed and/or by nitrogen retention and/or by animals' ability to avoid the negative effects of diseases or by the immune response of the subject.

Animal performance characteristics may include but are not limited to: body weight; weight gain; mass; body fat percentage; height; body fat distribution; growth; growth rate; egg size; egg weight; egg mass; egg laying rate; mineral absorption; mineral excretion, mineral retention; bone density; bone strength; feed conversion rate (FCR); average daily feed intake (ADFI); Average daily gain (ADG) retention and/or a secretion of any one or more of copper, sodium, phosphorous, nitrogen and calcium; amino acid retention or absorption; mineralization, bone mineralization carcass yield and carcass quality.

By “improved animal performance on one or more metric” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed comprising the feed additive composition or animal feed diet described herein as compared to a feed which does not comprise said feed additive composition. In some embodiments, the improvement in the one or more metric is at least about 1%, 2%, 3%, 4%, 5%, 6%, 7%, 8%, 9%, 105, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, or more (inclusive of percentages falling in-between these values) as compared to a feed which does not comprise said phytase-containing low trace mineral content feed additive compositions or animal feed diets described herein.

Preferably, by “improved animal performance” it is meant that there is increased feed efficiency and/or increased weight gain and/or reduced feed conversion ratio. As used herein, the term “feed efficiency” refers to the amount of weight gain in an animal that occurs when the animal is fed ad-libitum or a specified amount of food during a period of time.

By “increased feed efficiency” it is meant that the use of a feed additive composition according the present invention in feed results in an increased weight gain per unit of feed intake compared with an animal fed without said feed additive composition being present.

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

The improvement in performance parameters may be in respect to a control in which the feed used does not comprise a phytase.

The term Tibia ash refers to a quantification method for bone mineralization. This parameter gives indication if phosphorus is deficient (e.g. the content should be low in the phosphorus deficient negative control diets) or sufficient (e.g. the content in phytase treatments are comparable to a positive control diets that meeting phosphorus requirement in broilers)

The term “phosphorus deficient diet” refers to a diet in which the phosphorous level is not sufficient to satisfy the nutritional requirements of an animal, e.g., a feed formulated with phosphorus levels much lower than the recommended levels by the National Research Council (NRC) or broiler breeders. The animal feed contains lower levels of the mineral than required for optimal growth. If the diet lacks phosphorus, the calcium will also not be taken up by the animal. Excess Ca can lead to poor phosphorus (P) digestibility and contribute to the formation of insoluble mineral-phytate complexes. Both deficiency of P and Ca can cause reduced skeletal integrity, subnormal growth and ultimately weight loss.

The terms “mineralization” or “mineralization” encompass mineral deposition or release of minerals. Minerals may be deposited or released from the body of the animal. Minerals may be released from the feed. Minerals may include any minerals necessary in an animal diet, and may include calcium, copper, sodium, phosphorus, iron and nitrogen.

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g., the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses phosphorus digestibility, starch digestibility, fat digestibility, protein digestibility, and amino acid digestibility. Digestible phosphorus (P) can be defined as ileal digestible P which is the proportion of total P intake absorbed at the end of the ileum by an animal or the fecal digestible P which is the proportion of total P intake that is not excreted in the feces.

The term “survival” as used herein means the number of subjects remaining alive. The term “improved survival” is another way of saying “reduced mortality”.

The term “carcass yield” as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

The terms “carcass quality” and “meat quality” are used interchangeably and refers to the compositional quality (lean to fat ratio) as well as palatability factors such as visual appearance, smell, firmness, juiciness, tenderness, and flavor. For example, producing poultry that does not have a “woody breast.” The woody breast is a quality issue stemming from a muscle abnormality in a small percentage of chicken meat in the U.S. This condition causes chicken breast meat to be hard to the touch and often pale in color with poor quality texture. Woody breast does not create any health or food safety concerns for people and the welfare of the chicken itself is not negatively impacted.

An “increased weight gain” refers to an animal having increased body weight on being fed feed comprising a feed additive composition compared with an animal being fed a feed without said feed additive composition being present.

The terms “animal feed composition,” “feed”, “feedstuff,” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

As used herein the term “contacted” refers to the indirect or direct application of any of the engineered phytase polypeptides or fragments thereof (or composition comprising any of the engineered phytase polypeptides or fragments thereof) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment, the feed additive composition of the present invention is preferably admixed with the product (e.g. feedstuff). Alternatively, the feed additive composition may be included in the emulsion or raw ingredients of a feedstuff. For some applications, it is important that the composition is made available on or to the surface of a product to be affected/treated. This allows the composition to impart a performance benefit.

Any of the engineered phytase polypeptides or fragments thereof described herein (or composition comprising such engineered phytase polypeptides or fragments thereof) may be applied to intersperse, coat and/or impregnate a product (e.g. a diet that contains no or substantially no trace minerals or feedstuff or raw ingredients of a feedstuff) with a controlled amount of said enzyme.

In another aspect, the feed additive composition can be homogenized to produce a powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes. It will be understood that any of the engineered phytase polypeptides or fragments thereof (or composition comprising any of the engineered phytase polypeptides or fragments thereof) described herein are suitable for addition to any appropriate feed material.

In other embodiments, the granule may be introduced into a feed pelleting process wherein the feed pretreatment process may be conducted between 70° C. and 95° C. for up to several minutes, such as between 85° C. and 95° C.

In some embodiments, any of the engineered phytase polypeptides or fragments thereof can be present in the feed in the range of 1 ppb (parts per billion) to 10% (w/w) based on pure enzyme protein. In some embodiments, the engineered phytase polypeptides or fragments thereof are present in the feedstuff is in the range of 1-100 ppm (parts per million). A preferred dose can be 1-20 g of an engineered phytase polypeptide or fragment thereof per ton of feed product or feed composition or a final dose of 1-20 ppm engineered phytase polypeptide or fragment thereof in the final feed product.

Preferably, an engineered phytase polypeptide or fragment thereof is present in the feed should be at least about 50-10,000 FTU/kg corresponding to roughly 0.1 to 20 mg engineered phytase polypeptide or fragment thereof protein/kg.

Ranges can include, but are not limited to, any combination of the lower and upper ranges discussed above.

Formulations and/or preparations comprising any of the engineered phytase polypeptides or fragments thereof and compositions described herein may be made in any suitable way to ensure that the formulation comprises active phytase enzymes. Such formulations may be as a liquid, a dry powder or a granule which may be uncoated/unprotected or may involve the use of a thermoprotectant coating depending upon the processing conditions. As was noted above, the engineered phytase polypeptides and fragments thereof can be formulated inexpensively on a solid carrier without specific need for protective coatings and still maintain activity throughout the conditioning and pelleting process. A protective coating to provide additional thermostability when applied in a solid form can be beneficial for obtaining pelleting stability when required in certain regions where harsher conditions are used or if conditions warrant it, e.g., as in the case of super conditioning feed above 90° C.

Feed additive composition described herein can be formulated to a dry powder or granules as described in WO2007/044968 (referred to as TPT granules) or WO1997/016076 or WO1992/012645 (each of which is incorporated herein by reference).

In one embodiment the feed additive composition may be formulated to a granule for feed compositions comprising: a core; an active agent (for example, a phytase, such as any of the engineered phytase polypeptides disclosed herein); and at least one coating, the active agent of the granule retaining at least 50% activity, at least 60% activity, at least 70% activity, at least 80% activity after conditions selected from one or more of a) a feed pelleting process, b) a steam-heated feed pretreatment process, c) storage, d) storage as an ingredient in an unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60% w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch.

In other embodiments, the granule may be introduced into a feed pelleting process wherein the feed pretreatment process may be conducted between 70° C. and 95° C. for up to several minutes, such as between 85° C. and 95° C.

The feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule.

Alternatively, the composition is in a liquid formulation suitable for consumption preferably such liquid consumption contains one or more of the following: a buffer, salt, sorbitol and/or glycerol.

Also, the feed additive composition may be formulated by applying, e.g. spraying, the enzyme(s) onto a carrier substrate, such as ground wheat for example.

In one embodiment, the feed additive composition may be formulated as a premix. By way of example only the premix may comprise one or more feed components, such as one or more minerals and/or one or more vitamins.

In one embodiment a direct fed microbial (“DFM”) and/or an engineered phytase polypeptide or fragment thereof are formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate and mixtures thereof.

It should be noted that any of the engineered phytase polypeptides and fragments thereof may be useful in grain applications, e.g. processing of grains for non-food/feed application, e.g. ethanol production

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N. Y. (1991) provide one of skill with a general dictionary of many of the terms used with this disclosure.

The disclosure is further defined in the following Examples. It should be understood that these Examples, while indicating certain embodiments, are given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1: Efficacy of a Novel Consensus Bacterial 6-Phytase Variant to Replace Added Trace Minerals in a Commercial Corn-Soybean Meal-Based Diet Containing a Normal Level of Calcium

This Example shows the efficacy of broiler diets supplemented with a novel consensus bacterial 6-phytase variant (PhyG; SEQ ID NO:26) without exogenously added TM (Zn, Fe, Cu, Mn, Se). When fed to broilers, these diets maintained growth performance, tibia ash, tibia weight, liver and plasma TM concentrations at levels equivalent to or improved compared with a nutritionally adequate diet supplemented with TM at commercial levels.

### Materials and Methods

The study was carried out in accordance with the European Directive 2010/63/EU and the regulations in force in the Netherlands for the care and use of animals in research. Experimental protocols and procedures were evaluated and approved by the Ethical Committee on Animal Experiments (Ethische Toetsing Dierproeven) of Schothorst Feed Research (Lelystad, the Netherlands).

Experimental design, birds and housing: The experiment was carried out as a randomized complete block design with 12 dietary treatments, 8 replicate floor-pens (each pen 2 m²) and 35 birds/pen. A total of 3,360 Ross 308 male broilers were assigned on day-of-hatch to pens so that each pen contained birds of approximately equal average bird weight. Pens were housed in a broiler house containing wood shavings as bedding. Temperature was maintained initially at 34° C. and gradually reduced to 22° C. on d 24, then to 21° C. on d 29, 20° C. on d 32 and 19° C. on d 35. The lighting regime was LD 23:1 h on d 1 and thereafter 3L:1D:12L:4D: 3L:1D. Diets were fed as pelleted dicts (pelleting temperature≤80° C.) ad lib in all phases.

Dietary treatments: Treatment diets were formulated in three phases: 1-10, 10-20 and 20-35 d of age as starter, grower and finisher phases, respectively. Treatments comprised:
1) CON1: control diet, formulated to meet the nutrient requirements for broilers applicable in the Netherlands (CVB, 2021) but without added TM (Zn, Cu, Fe, Mn and Se);
2) CON1 plus supplemental Zn, Cu, Fe, Mn in sulphate-based form and Se as Na₂SeO₃, added at a ‘low’ level (reduced by 50% compared to commercial levels) during all phases;
3) CON1 plus supplemental Zn, Cu, Fe, Mn in sulphate-based form and Se as Na₂SeO₃, added at a ‘high’ level (commercial level) during all phases;
4) CON1 plus supplemental Zn, Cu, Mn in oxide-based form and Se as Na₂SeO₃ and Fe in sulphate-based form, added at a ‘low’ level during all phases;
5) CON1 plus supplemental Zn, Cu, Mn in oxide-based form and Se as Na₂SeO₃ and Fe in sulphate-based form, added at a ‘high’ level during all phases;
6) CON1 plus supplemental Zn, Fe, Cu, Mn in organic-based form and Se as Na₂SeO₃, added at a ‘low’ level during all phases;
7) CON2: control diet, as CON1 but reduced in Ca and digestible P (to account for the expected activity of PhyG phytase) and containing PhyG added at 2,000, 1,500 and 1,000 FTU/kg in starter, grower and finisher phases, respectively;
8) CON2 plus supplemental Zn, Fe, Cu, Mn in sulphate-based form and Se as Na₂SeO₃ added at a ‘low’ level during all phases and supplemented with PhyG as in 7;
9) CON2 plus supplemental Zn, Fe, Cu, Mn in sulphate-based form and Se as Na₂SeO₃ added at a ‘high’ level during all phases and supplemented with PhyG as in 7;
10) CON2 plus supplemental Zn, Cu and Mn in oxide-based form and Se as Na₂SeO₃ and Fe in sulphate-based form, added at a ‘low’ level during all phases and supplemented with PhyG as in 7;
11) CON2 plus supplemental Zn, Cu and Mn in oxide-based form and Se as Na₂SeO₃ and Fe in sulphate-based form, added at a ‘high’ level during all phases and supplemented with PhyG as in 7;
12) CON2 plus supplemental Zn, Cu, Fe and Mn in organic-based form and Se as Na₂SeO₃, added at a ‘low’ level during all phases and supplemented with PhyG as in 7.

The concentrations of the individual TM added to each treatment are given in Table 1. The ingredient and calculated nutrient composition of the basal CON1 and CON2 dicts is given in Table 2 and the analyzed TM content of the diets is given in Table 3.

TABLE 1
Dietary treatment details

| | | Added trace minerals (mg/kg feed)² | | | | | | |
|---|---|---|---|---|---|---|---|---|
| | Phytase | | Level | | | | | |
| Treatment | (+/−)¹ | Source | (Low/High)³ | Zn | Cu | Fe⁴ | Mn | Se⁵ |
| 1 (=CON1) | − | — | — | — | — | — | — | — |
| 2 | − | sulphate-based | low | 10 | 3 | 15 | 10 | 0.15 |
| 3 | − | sulphate-based | high | 20 | 6 | 30 | 20 | 0.15 |
| 4 | − | oxide-based | low | 15 | 3 | 15 | 10 | 0.15 |
| 5 | − | oxide-based | high | 30 | 6 | 30 | 20 | 0.15 |
| 6 | − | organic-based | low | 10 | 3 | 15 | 10 | 0.15 |
| 7 (=CON2) | + | — | — | — | — | — | — | — |
| 8 | + | sulphate-based | low | 10 | 3 | 15 | 10 | 0.15 |
| 9 | + | sulphate-based | high | 20 | 6 | 30 | 20 | 0.15 |
| 10 | + | oxide-based | low | 15 | 3 | 15 | 10 | 0.15 |
| 11 | + | oxide-based | high | 30 | 6 | 30 | 20 | 0.15 |
| 12 | + | organic-based | low | 10 | 3 | 15 | 10 | 0.15 |

¹Included at 2,000, 1,500 and 1,000 FTU/kg in starter (d 1-10), grower (d 10-20) and finisher (d 20-35) phases, respectively.
²Values for individual trace minerals represent the concentrations expected to be present as mg per kilogram of final diet.
³‘Low’: reduced by 50% vs a commercial level of supplementation; ‘high’: a commercially relevant level of supplementation.
⁴FeSO₄ was used in place of FeO in treatments 4,5,10 and 11, for regulatory reasons.
⁵Se was added in the form of Na₂SeO₃ in all treatments.

TABLE 2
Ingredient and calculated nutrient composition of the basal diets in Example 1

| | CON1¹ | | | CON2² | | |
|---|---|---|---|---|---|---|
| | Starter | Grower | Finisher | Starter | Grower | Finisher |
| Item | (1-10 d) | (10-20 d) | (20-35 d) | (1-10 d) | (10-20 d) | (20-35 d) |
| Ingredients, % as fed | | | | | | |
| Corn | 58.2 | 57.7 | 63.2 | 58.2 | 57.7 | 63.2 |
| Soybean meal | 28.6 | 29.4 | 18.28 | 28.7 | 29.4 | 18.28 |
| Rice bran | 2.82 | 3.23 | — | 2.83 | 3.23 | — |
| Corn gluten meal | 3.00 | 1.00 | — | 3.00 | 1.00 | — |
| Sunflower seed meal | — | — | 6.00 | — | — | 6.00 |
| Rapeseed meal | — | — | 3.60 | — | — | 3.60 |
| Soybean oil | 2.01 | 3.40 | 2.75 | 2.01 | 3.44 | 2.75 |
| Limestone | 1.23 | 1.11 | 1.04 | 1.10 | 0.97 | 0.90 |
| Monocalcium phosphate | 1.61 | 1.41 | 1.11 | 0.60 | 0.44 | 0.22 |
| Silicate | — | — | — | 1.15 | 1.10 | 1.03 |
| Lard | — | 0.19 | 2.10 | — | 0.19 | 2.10 |
| Salt | 0.20 | 0.07 | 0.26 | 0.20 | 0.07 | 0.26 |
| L-lysine HCl | 0.42 | 0.26 | 0.31 | 0.42 | 0.26 | 0.31 |
| DL-methionine | 0.31 | 0.27 | 0.17 | 0.31 | 0.27 | 0.17 |
| L-threonine | 0.11 | 0.09 | 0.06 | 0.11 | 0.09 | 0.06 |
| L-tryptophan | 0.03 | 0.01 | 0.01 | 0.03 | 0.01 | 0.01 |
| L-arginine | 0.12 | — | — | 0.12 | — | — |
| L-valine | 0.07 | 0.01 | 0.01 | 0.07 | 0.01 | 0.01 |
| Titanium dioxide | — | 0.35 | — | — | 0.35 | — |
| Sodium bicarbonate | 0.20 | 0.39 | 0.10 | 0.20 | 0.39 | 0.10 |
| Vitamin premix³ | 0.50 | 0.50 | 0.50 | 0.50 | 0.50 | 0.50 |
| Test premix (corn only in | 0.50 | 0.50 | 0.50 | 0.50 | 0.50 | 0.50 |
| CON1) | | | | | | |
| Sacox | 0.01 | 0.01 | — | 0.01 | 0.01 | |
| Phytase, FTU/kg | — | — | — | 2,000 | 1,500 | 1,000 |
| Total | 100.0 | 100.0 | 100.0 | 100.0 | 100.0 | 100.0 |
| Chemical composition, % as | | | | | | |
| is (unless otherwise stated) | | | | | | |
| ME, kcal/kg | 2,900 | 2,975 | 3,075 | 2,900 | 2,975 | 3,075 |
| Moisture | 11.7 | 11.7 | 11.6 | 11.7 | 11.7 | 11.6 |
| Ash | 6.05 | 5.99 | 5.33 | 6.05 | 5.99 | 5.33 |
| Crude protein | 21.1 | 19.8 | 17.8 | 21.1 | 19.8 | 17.8 |
| Crude fat | 5.30 | 6.83 | 7.96 | 5.30 | 6.83 | 7.96 |
| Crude fiber | 2.40 | 2.41 | 2.92 | 2.40 | 2.41 | 2.92 |
| Calcium | 0.92 | 0.84 | 0.77 | 0.69 | 0.62 | 0.56 |
| Phosphorus (P) | 0.72 | 0.67 | 0.61 | 0.50 | 0.46 | 0.42 |
| Retainable phosphorus | 0.43 | 0.39 | 0.33 | 0.24 | 0.21 | 0.17 |
| Phytate-P | 0.25 | 0.25 | 0.25 | 0.25 | 0.25 | 0.25 |
| Zinc, mg/kg | 28 | 28 | 30 | 27 | 27 | 29 |
| Iron, mg/kg | 150 | 150 | 148 | 107 | 109 | 110 |
| Copper, mg/kg | 5.4 | 5.0 | 5.8 | 5.4 | 5.0 | 5.8 |
| Manganese, mg/kg | 28 | 27 | 24 | 23 | 22 | 19 |
| Selenium, mg/kg | 0.10 | 0.10 | 0.10 | 0.10 | 0.10 | 0.10 |

¹Formulated to meet the nutrient requirements for broilers applicable in the Netherlands (CVB, 2021) but without added trace minerals.
²Reduced in Ca and dig P vs. CON1 (to account for the expected Ca and dig P contribution of the added phytases), without trace mineral supplementation but with addition of phytase (added at 2,000, 1,500 and 1,000 FTU/kg in starter, grower and finisher phases respectively).
³Supplied per kilogram of diet: vitamin A (retinyl acetate 3a672a, 10,000 IU), vitamin D3 (3a671, 2,500 IU), vitamin E (dl-α-tocopherol 3a700, 50 IU), vitamin K3 (menadione 3a711, 1.5 mg), vitamin B1 (thiamine mononitrate 3a821, 2.0 mg), riboflavin (7.5 mg), D-pantothenic acid (12 mg), vitamin B6 (3.5 mg), vitamin B12 (0.020 mg), niacin (35 mg), folic acid (1.0 mg), biotin (0.20 mg), choline (choline chloride, 460 mg) and I (KI, 0.8 mg).

TABLE 3
Analyzed trace minerals content of the experimental diets in Example 1.

| | | Treatment No.¹ | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Ca, g/kg | Starter | 8.48 | — | — | — | — | — | 6.94 | — | — | — | — | — |
| | Grower | 9.17 | — | — | — | — | — | 6.99 | — | — | — | — | — |
| | Finisher | 7.55 | — | — | — | — | — | 5.38 | — | — | — | — | — |
| P, g/kg | Starter | 8.25 | — | — | — | — | — | 5.41 | — | — | — | — | — |
| | Grower | 7.01 | — | — | — | — | — | 4.79 | — | — | — | — | — |
| | Finisher | 6.62 | — | — | — | — | — | 4.23 | — | — | — | — | — |
| Zn, mg/kg | Starter | 27 | 36 | 46 | 42 | 57 | 37 | 27 | 35 | 43 | 44 | 57 | 38 |
| | Grower | 31 | 40 | 48 | 44 | 60 | 39 | 27 | 38 | 47 | 43 | 62 | 41 |
| | Finisher | 31 | 40 | 52 | 45 | 62 | 40 | 29 | 41 | 53 | 45 | 62 | 40 |
| Fe, mg/kg | Starter | 172 | 173 | 190 | 174 | 203 | 174 | 118 | 120 | 137 | 136 | 135 | 117 |
| | Grower | 168 | 178 | 206 | 193 | 221 | 172 | 135 | 140 | 162 | 149 | 158 | 130 |
| | Finisher | 159 | 169 | 176 | 172 | 178 | 162 | 122 | 123 | 144 | 116 | 146 | 135 |
| Cu, mg/kg | Starter | 5.8 | 8 | 12 | 8.8 | 12 | 8.5 | 5.7 | 8.7 | 10 | 8.6 | 12 | 8.7 |
| | Grower | 5 | 6.1 | 9.9 | 7.1 | 10 | 7.3 | 5 | 6.9 | 8.9 | 7.3 | 10 | 6.3 |
| | Finisher | 5.7 | 8.6 | 11 | 8.8 | 11 | 7.5 | 5 | 7.1 | 12 | 7.7 | 11 | 8.4 |
| Mn, | Starter | 33 | 39 | 47 | 40 | 52 | 39 | 25 | 34 | 43 | 31 | 40 | 34 |
| mg/kg | Grower | 32 | 41 | 51 | 39 | 50 | 42 | 26 | 35 | 48 | 32 | 47 | 35 |
| | Finisher | 25 | 34 | 41 | 31 | 61 | 33 | 19 | 27 | 37 | 30 | 36 | 28 |
| Se, mg/kg | Starter | 0.14 | 0.23 | 0.27 | 0.27 | 0.3 | 0.27 | 0.11 | 0.21 | 0.27 | 0.29 | 0.3 | 0.27 |
| | Grower | 0.16 | 0.28 | 0.28 | 0.28 | 0.25 | 0.24 | 0.14 | 0.29 | 0.29 | 0.26 | 0.33 | 0.34 |
| | Finisher | 0.1 | — | 0.22 | 0.23 | 0.25 | 0.23 | 0.11 | 0.23 | 0.27 | 0.27 | 0.26 | 0.27 |
| Phytase | Starter | 89 | 150 | — | — | — | — | 1,812 | 2,188 | 2,159 | 2,073 | 1,815 | 2,078 |
| activit [symbol] | Grower | 182 | 154 | — | — | — | — | 1,529 | 1,643 | 1,597 | 1,481 | 1,264 | 1,521 |
| FTU, kg | Finisher | 152 | 148 | — | — | — | — | 1,184 | 923 | 1,043 | 1,079 | 1,377 | 1,200 |

*not reported.
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.
⁴S, starter (1 to 10 d of age); G, grower (10 to 20 d of age): F, finisher (20 to 35 d of age). [symbol]
indicates data missing or illegible when filed

Sampling, measurements and statistical analysis: At each of 10, 20 and 35 d of age, 4 birds/pen were euthanized. Blood was collected from 2 birds/pen, plasma extracted and one of the two samples per pen was analyzed for TM, livers from 2 birds/pen were collected, weighed and one of the two samples per pen was analyzed for TM. Tibias (left) from 4 birds/pen were extracted, pooled, de-fatted, ashed and analyzed for TM. Trace minerals in the liver were analyzed by inductively coupled plasma-optical emission spectrometry (ICP-OES) and in plasma were analyzed by atomic absorption spectrometry (AAS), both in duplicate. Data were analyzed by 2-way ANOVA (2×6 factorial design with two levels of phytase supplementation and 6 levels of trace mineral supplementation). Treatment means were separated using Tukey's HSD test. P<0.05 was considered statistically significant. 0.05≤P<0.1 was considered a tendency.

### Results

Growth performance: The results for body weight (BW), BW gain (BWG), feed intake (FI) and feed conversion ratio (FCR) by phase are presented in Tables 4-6.

Even without supplemental TM, PhyG phytase supplemented to CON 2 (treatment 7) maintained growth performance at a level that was statistically equivalent to, or improved, compared with that of the CON1 treatments supplemented with TM (treatments 2-6). This effect was seen in all performance measures (BW, BWG, FI and FCR), during all individual phases and overall (1-35 d of age, data not shown) (Tables 4, 5 and 6). The improvements in FCR with the phytase (independent of effects of TM supplementation), that were evident during all individual phases, suggest that the increased weight gain in birds supplemented with phytase were not solely driven by increased FI, but also by increased utilization of TM per unit of ingested feed.

Without phytase, TM supplementation improved all measures of growth performance (increased BW, BW gain, FI and reduced FCR) during starter phase (1-10 d of age), but had less effect during grower (10-20 d of age) and finisher (20-35 d of age) phases; during grower phase TM supplementation increased BW, BWG and FI but had no effect on FCR, and during finisher phase TM supplementation had no effect on BWG, FI or FCR, although final BW at d 35 of age was higher in the majority of the TM-supplemented treatments (exception: treatment low, oxide-based TM treatment 4) compared with the CON1 diet without supplemental TM (treatment 1). The effects of TM supplementation on growth performance were evident regardless of TM source or dose level. With phytase, TM supplementation provided no further benefit in terms of growth performance (all measures, all phases), because of the overriding beneficial effect of the phytase (treatments 8-12 compared with 7 in Tables 4, 5 and 6).

Mortality levels were consistently low overall (1-35 d of age, below 2.6%, on average 1.3%) and were unaffected by treatment.

TABLE 4
Effect of supplementation with trace minerals (low or high), phytase
or both, on growth performance during 1-10 d; 2-way ANOVA

| | | Trace mineral | | | | | |
|---|---|---|---|---|---|---|---|
| | Phytase | supplementation³ | | d 10 BW, | BWG, | FI, | FCR, |
| Treatment no.¹ | (+/−)² | Source | Level | g/bird | g/bird | g/bird | g:g |
| Treatment means | | | | | | | |
| 1 | − | — | — | 279^{c} | 237^{e} | 245^{c} | 1.033^{a} |
| 2 | − | sulphate-based | Low | 303^{b} | 266^{cd} | 271^{ab} | 1.017^{b} |
| 3 | − | sulphate-based | High | 316^{ab} | 274^{abcd} | 276^{ab} | 1.007^{bcde} |
| 4 | − | oxide-based | Low | 307^{b} | 265^{d} | 268^{b} | 1.014^{bcd} |
| 5 | − | oxide-based | High | 313^{ab} | 271^{abcd} | 272^{ab} | 1.003^{cdef} |
| 6 | − | organic-based | Low | 308^{b} | 266^{d} | 270^{ab} | 1.015^{bc} |
| 7 | + | — | — | 311^{b} | 267^{bcd} | 269^{b} | 1.000^{ef} |
| 8 | + | sulphate-based | Low | 318^{ab} | 279^{abc} | 276^{ab} | 0.988f |
| 9 | + | sulphate-based | High | 319^{ab} | 280^{ab} | 278^{ab} | 0.993^{f} |
| 10 | + | oxide-based | Low | 325^{a} | 283^{a} | 282^{a} | 0.995^{ef} |
| 11 | + | oxide-based | High | 315^{ab} | 277^{abcd} | 277^{ab} | 1.001^{def} |
| 12 | + | organic | Low | 318^{ab} | 276^{abcd} | 276^{ab} | 0.999^{ef} |
| Main effects | | | | | | | |
| Phytase | − | | | 305 | 263 | 267.0 | 1.015 |
| | + | | | 318 | 277 | 276.1 | 0.996 |
| Trace minerals | | — | | 295 | 253 | 256.8 | 1.017 |
| | | sulphate-based | Low | 313 | 273 | 273.3 | 1.003 |
| | | sulphate-based | High | 318 | 277 | 277.0 | 1.000 |
| | | oxide-based | Low | 316 | 274 | 274.9 | 1.004 |
| | | oxide-based | High | 314 | 274 | 274.7 | 1.002 |
| | | organic | Low | 313 | 271 | 272.7 | 1.007 |

| | | | | |
|---|---|---|---|---|
| SEM | 3.05 | 2.55 | 2.55 | 0.003 |
| P-value, phytase | <0.001 | <0.001 | <0.001 | <0.001 |
| P-value trace minerals | <0.001 | <0.001 | <0.001 | <0.001 |
| P-value phytase × trace minerals | <0.001 | <0.001 | <0.001 | <0.001 |

^{a,b,c,d}Means within each column grouping with uncommon superscripts are significantly different at P < 0.05.
¹Treatments 1-6 were based on CON1, treatments 7-12 were based on CON2 (see Table 2).
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

TABLE 5
Effect of supplementation with trace minerals (low or high), phytase
or both, on growth performance during 10-20 d; 2-way ANOVA

| | | Trace mineral | | | | | |
|---|---|---|---|---|---|---|---|
| | Phytase | supplementation³ | | d 20 BW, | BWG, | FI, | FCR, |
| Treatment no.¹ | (+/−)² | Source | Level | g/bird | g/bird | g/bird | g:g |
| Treatment means | | | | | | | |
| 1 | − | — | — | 937^{f} | 658^{d} | 829^{d} | 1.259 |
| 2 | − | sulphate-based | Low | 1,017^{de} | 706^{bc} | 886^{abc} | 1.255 |
| 3 | − | sulphate-based | High | 1,031^{abcde} | 715^{abc} | 894^{abc} | 1.250 |
| 4 | − | oxide-based | Low | 1,001^{e} | 694^{c} | 867^{c} | 1.250 |
| 5 | − | oxide-based | High | 1,023^{bcde} | 710^{abc} | 891^{bc} | 1.255 |
| 6 | − | organic-based | Low | 1,014^{de} | 711^{abc} | 885^{abc} | 1.246 |
| 7 | + | — | — | 1,033^{abcde} | 723^{ab} | 885^{abc} | 1.224 |
| 8 | + | sulphate-based | Low | 1,036^{abcde} | 714^{abc} | 880^{bc} | 1.232 |
| 9 | + | sulphate-based | High | 1,061^{a} | 736^{a} | 905^{ab} | 1.230 |
| 10 | + | oxide-based | Low | 1,056^{ab} | 736^{a} | 914^{a} | 1.242 |
| 11 | + | oxide-based | High | 1,051^{abc} | 736^{a} | 897^{abc} | 1.219 |
| 12 | + | organic | Low | 1,045^{abcd} | 728^{ab} | 893^{abc} | 1.227 |
| Main effects | | | | | | | |
| Phytase | − | | | 1,004 | 699 | 875 | 1.253^{a} |
| | + | | | 1,047 | 729 | 896 | 1.229^{b} |
| Trace minerals | | — | | 985 | 691 | 857 | 1.242 |
| | | sulphate-based | Low | 1,027 | 710 | 883 | 1.243 |
| | | sulphate-based | High | 1,046 | 725 | 900 | 1.240 |
| | | oxide-based | Low | 1,028 | 715 | 891 | 1.246 |
| | | oxide-based | High | 1,037 | 723 | 894 | 1.237 |
| | | organic | Low | 1,030 | 719 | 889 | 1.236 |

| | | | | |
|---|---|---|---|---|
| SEM | <0.001 | <0.001 | <0.001 | <0.001 |
| P-value, phytase | <0.001 | <0.001 | <0.001 | <0.001 |
| P-value trace minerals | <0.001 | <0.001 | <0.001 | 0.453 |
| P-value phytase × trace minerals | <0.001 | <0.001 | <0.001 | 0.097 |

^{a,b,c,d,e}Means within each column grouping with uncommon superscripts are significantly different at P < 0.05.
¹Treatments 1-6 were based on CON1, treatments 7-12 were based on CON2 (see Table 1).
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

TABLE 6
Effect of supplementation with trace minerals (low or high), phytase
or both, on growth performance during 20-35 d; 2-way ANOVA

| | | Trace mineral | | | | | |
|---|---|---|---|---|---|---|---|
| | Phytase | supplementation³ | | d 35 BW, | BWG, | FI, | FCR, |
| Treatment no.¹ | (+/−)² | Source | Level | g/bird | g/bird | g/bird | g:g |
| Treatment means | | | | | | | |
| 1 | − | — | — | 2,471^{b} | 1,562 | 2,431 | 1.558 |
| 2 | − | sulphate-based | Low | 2,625^{a} | 1,608 | 2,521 | 1.568 |
| 3 | − | sulphate-based | High | 2,677^{a} | 1,646 | 2,538 | 1.544 |
| 4 | − | oxide-based | Low | 2,591^{ab} | 1,591 | 2,483 | 1.563 |
| 5 | − | oxide-based | High | 2,633^{a} | 1,610 | 2,496 | 1.551 |
| 6 | − | organic-based | Low | 2,640^{a} | 1,626 | 2,500 | 1.538 |
| 7 | + | — | — | 2,645^{a} | 1,612 | 2,510 | 1.557 |
| 8 | + | sulphate-based | Low | 2,671^{a} | 1,631 | 2,500 | 1.532 |
| 9 | + | sulphate-based | High | 2,676^{a} | 1,621 | 2,473 | 1.527 |
| 10 | + | oxide-based | Low | 2,724^{a} | 1,682 | 2,593 | 1.542 |
| 11 | + | oxide-based | High | 2,694^{a} | 1,648 | 2,504 | 1.520 |
| 12 | + | organic | Low | 2,699^{a} | 1,654 | 2,518 | 1.522 |
| Main effects | | | | | | | |
| Phytase | − | | | 2,606 | 1,607^{a} | 2,495 | 1.554^{a} |
| | + | | | 2,685 | 1,641^{b} | 2,516 | 1.533^{b} |
| Trace minerals | | — | | 2,558 | 1,587 | 2,470 | 1.558 |
| | | sulphate-based | Low | 2.648 | 1,620 | 2,510 | 1.550 |
| | | sulphate-based | High | 2,676 | 1,633 | 2,505 | 1.535 |
| | | oxide-based | Low | 2,658 | 1,636 | 2,538 | 1.553 |
| | | oxide-based | High | 2,663 | 1,629 | 2,500 | 1.535 |
| | | organic | Low | 2,670 | 1,640 | 2,509 | 1.530 |

| | | | | |
|---|---|---|---|---|
| SEM | 29.15 | 24.48 | 31.06 | 0.012 |
| P-value, phytase | <0.001 | 0.017 | 0.238 | 0.004 |
| P-value trace minerals | 0.001 | 0.295 | 0.462 | 0.116 |
| P-value phytase × trace minerals | 0.045 | 0.317 | 0.074 | 0.731 |

^{a,b}Means within each column grouping with uncommon superscripts are significantly different at P < 0.05.
¹Treatments 1-6 were based on CON1, treatments 7-12 were based on CON2 (see Table 1).
²Included at 2,000, 1,500 and 1,000 FTU/kg in starter (d 0-10), grower (d 10-20) and finisher (d 20-35) phases, respectively.

Bone mineralization: Tibia Zn was negatively affected by the removal of TM in the absence of exogenous phytase (treatment 1 vs. treatments 2-6). Even without supplemental TM, phytase supplemented to CON 2 (treatment 7) improved tibia Zn compared with that of any of the CON1 treatments (i.e. supplemented with TM or not, treatments 1-6; Table 7); tibia Zn in CON2+phytase without TM (treatment 7) was 77% higher than in treatment 1, and 7-25% higher than in treatments 2-6. As seen for growth performance, there was no extra benefit of TM supplementation on tibia Zn over and above that delivered by the phytase, as indicated by the significant interaction between phytase and TM supplementation in their effects on tibia Zn and the differences between the corresponding means for treatments 7 to 12 in Table 7.

TABLE 7
Effect of supplementation with trace minerals (low or high), phytase or
both, on tibia ash and trace mineral content at 20 d of age; 2-way ANOVA

| | | Trace mineral | | Zn, | Fe, | Cu, | Mn, |
|---|---|---|---|---|---|---|---|
| | Phytase | supplementation³ | | mg/kg | mg/kg | mg/kg | mg/kg |
| Treatment no.¹ | (+/−)² | Source | Level | DM | DM | DM | DM |
| Treatment means | | | | | | | |
| 1 | − | — | — | 170.0^{f} | 257.5 | 2.91 | 5.73 |
| 2 | − | sulphate-based | Low | 241.3^{a} | 260.6 | 3.03 | 5.90 |
| 3 | − | sulphate-based | High | 270.9^{c} | 243.1 | 2.73 | 6.11 |
| 4 | − | oxide-based | Low | 264.6^{cd} | 243.2 | 2.30 | 6.36 |
| 5 | − | oxide-based | High | 281.6^{bc} | 255.1 | 2.75 | 6.47 |
| 6 | − | organic-based | Low | 249.1^{de} | 251.1 | 2.66 | 6.21 |
| 7 | + | — | — | 300.3^{a} | 253.7 | 2.72 | 6.46 |
| 8 | + | sulphate-based | Low | 295.8^{ab} | 262.6 | 2.47 | 7.73 |
| 9 | + | sulphate-based | High | 293.1^{ab} | 240.4 | 2.36 | 8.21 |
| 10 | + | oxide-based | Low | 291.4^{ab} | 268.9 | 2.54 | 7.61 |
| 11 | + | oxide-based | High | 303.4^{a} | 259.2 | 2.28 | 7.84 |
| 12 | + | organic | Low | 295.8^{ab} | 246.6 | 2.81 | 7.68 |
| Main effects | | | | | | | |
| Phytase | − | | | 246.3 | 251.8 | 2.73 | 6.13^{a} |
| | + | | | 296.6 | 255.2 | 2.53 | 7.59^{b} |
| Trace minerals | | — | | 235.2 | 255.6 | 2.81 | 6.10^{b} |
| | | sulphate-based | Low | 268.5 | 261.6 | 2.75 | 6.82^{b} |
| | | sulphate-based | High | 282.0 | 241.7 | 2.54 | 7.16^{b} |
| | | oxide-based | Low | 278.0 | 256.0 | 2.42 | 6.98^{a} |
| | | oxide-based | High | 292.5 | 257.2 | 2.55 | 7.16^{a} |
| | | organic | Low | 272.4 | 248.8 | 2.73 | 6.94^{a} |

| | | | | |
|---|---|---|---|---|
| SEM | 3.82 | 11.43 | 0.31 | 0.22^{a} |
| P-value, phytase | <0.001 | 0.430 | 0.622 | <0.001 |
| P-value trace minerals | <0.001 | 0.842 | 0.944 | <0.001 |
| P-value phytase × trace minerals | <0.001 | 0.815 | 0.356 | 0.080 |

^{a,b,c,d}Means within each column grouping with uncommon superscripts are significantly different at P < 0.05.
¹Treatments 1-6 were based on CON1, treatments 7-12 were based on CON2 (see Table 1).
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

Trace minerals in the liver: Phytase or trace mineral supplementation had no effect on liver weight at 20 d of age (Table 8). Even without supplemental TM, phytase supplemented to CON2 (treatment 7) maintained liver Zn and Fe at levels that were improved (vs. CON1) or equivalent to that of the CON1 treatments supplemented with TM (vs. treatments 2-6; Table 8). For liver Zn and Fe, similar interactions were evident to those seen for tibia Zn; there was no extra benefit of TM supplementation on liver Zn or Fe over and above that delivered by the phytase (treatments 8-12 compared with treatment 7), whilst in the absence of phytase there was some evidence of increased liver Zn with TM supplementation (e.g. liver Zn was significantly higher in treatment 3 vs treatment 1, whilst a numerical increase was observed for liver Zn and

Fe in treatment 2-6 vs treatment 1, Table 8).

TABLE 8
Effect of supplementation with trace minerals (low or high), phytase
or both, on liver trace mineral content at 20 d of age; 2-way ANOVA

| | | Trace mineral | | Liver | | | | |
|---|---|---|---|---|---|---|---|---|
| Treatment | Phytase | supplementation³ | | weight, | Zn, | Fe, | Cu, | Mn, |
| no.¹ | (+/−)² | Source | Level | % BW | mg/kg | mg/kg | mg/kg | mg/kg |

Treatment means

| | | | | | | | | |
|---|---|---|---|---|---|---|---|---|
| 1 | − | — | — | 3.14 | 16.50^{b} | 73.1^{b} | 2.23 | 1.86 |
| 2 | − | sulphate-based | Low | 3.14 | 17.75^{ab} | 80.7^{ab} | 2.36 | 2.05 |
| 3 | − | sulphate-based | High | 3.14 | 18.38^{a} | 76.1^{b} | 2.39 | 2.06 |
| 4 | − | oxide-based | Low | 3.10 | 17.63^{ab} | 93.4^{ab} | 2.25 | 2.11 |
| 5 | − | oxide-based | High | 3.04 | 18.00^{ab} | 80.6^{ab} | 2.39 | 2.21 |
| 6 | − | organic-based | Low | 3.18 | 17.75^{ab} | 76.1^{b} | 2.41 | 1.99 |
| 7 | + | — | — | 3.08 | 18.8^{a} | 113.1^{a} | 2.44 | 2.25 |
| 8 | + | sulphate-based | Low | 3.10 | 18.25^{a} | 97.1^{ab} | 2.44 | 2.20 |
| 9 | + | sulphate-based | High | 3.15 | 18.00^{ab} | 110.9^{a} | 2.46 | 2.36 |
| 10 | + | oxide-based | Low | 3.05 | 18.13^{ab} | 85.7^{ab} | 2.39 | 2.29 |
| 11 | + | oxide-based | High | 3.13 | 18.71^{a} | 98.9^{ab} | 2.49 | 2.43 |
| 12 | + | organic | Low | 3.02 | 18.63^{a} | 113.0^{a} | 2.43 | 2.43 |

Main effects

| | | | | | | | | |
|---|---|---|---|---|---|---|---|---|
| Phytase | − | | | 3.12 | 17.67 | 80.0 | 2.34^{a} | 2.05^{a} |
| | + | | | 3.09 | 18.41 | 103.1 | 2.44^{b} | 2.33^{b} |
| Trace minerals | | — | | 3.11 | 17.63 | 93.1 | 2.33 | 2.06^{b} |
| | | sulphate-based | Low | 3.12 | 18.00 | 88.9 | 2.40 | 2.13^{ab} |
| | | sulphate-based | High | 3.14 | 18.19 | 93.5 | 2.43 | 2.21^{ab} |
| | | oxide-based | Low | 3.08 | 17.88 | 89.5 | 2.32 | 2.20^{ab} |
| | | oxide-based | High | 3.08 | 18.36 | 89.7 | 2.44 | 2.32^{a} |
| | | organic | Low | 3.10 | 18.19 | 94.6 | 2.42 | 2.21^{ab} |

| | | | | | |
|---|---|---|---|---|---|
| SEM | 0.066 | 0.366 | 7.38 | 0.063 | 0.088 |
| P-value, phytase | 0.356 | 0.001 | <0.001 | 0.006 | <0.001 |
| P-value trace minerals | 0.925 | 0.407 | 0.954 | 0.263 | 0.077 |
| P-value phytase × trace minerals | 0.555 | 0.025 | 0.017 | 0.710 | 0.476 |

^{a,b,c,d}Means within each column grouping with uncommon superscripts are significantly different at P < 0.05.
¹Treatments 1-6 based on CON1, treatments 7-12 based on CON2 (see Table 1).
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

Trace minerals in plasma: Plasma mineral concentrations were less influenced by phytase or trace mineral supplementation than tibia or liver trace mineral concentrations, with the exception of plasma Se (Table 9).

As before, even without supplemental TM, phytase supplemented to CON2 (treatment 7) maintained plasma Zn, Fe, Cu and Mn at levels that were equivalent to or improved compared with that of any of the CON1 treatments supplemented with TM (treatments 2-6; Table 9). For plasma Zn, there was a similar interaction evident to that seen for tibia Zn; there was no extra benefit of trace mineral supplementation on liver Zn or Fe over and above that delivered by the phytase (treatments 8-12 compared with treatment 7), whilst in the absence of phytase there was some evidence of increased plasma Zn and Se with TM supplementation (treatments 2-6 compared with treatment 1).

For plasma Se, unlike the other trace minerals, TM supplementation had a very substantial elevating effect (+757 to +893% in treatments 2-6 vs. treatment 1; Table 9), whereas phytase supplementation had no independent effect. An interaction was however present, whereby in the presence of phytase, the plasma Se response to TM appeared to be slightly lower than in the absence of phytase (+652 to +723% in treatments 8-12 vs treatment 7; Table 9).

TABLE 9
Effect of supplementation with trace minerals (low or high) or phytase
or both, on plasma trace mineral content at 20 d of age; 2-way ANOVA

| | | Trace mineral | | | | | | |
|---|---|---|---|---|---|---|---|---|
| Treatment | Phytase | supplementation³ | | Zn, | Fe, | Cu, | Mn, | Se, |
| no.¹ | (+/−)² | Source | Level | μmol/L | μmol/L | μmol/L | μg/L | μg/L |

Treatment means

| | | | | | | | | |
|---|---|---|---|---|---|---|---|---|
| 1 | − | — | — | 16.95^{b} | 15.00 | 1.23 | 47.73 | 17.3^{b} |
| 2 | − | sulphate-based | Low | 20.86^{a} | 15.33 | 1.08 | 34.98 | 171.9^{a} |
| 3 | − | sulphate-based | High | 21.06^{a} | 13.95 | 0.70 | 37.54 | 148.2^{a} |
| 4 | − | oxide-based | Low | 20.54^{ab} | 13.75 | 0.65 | 38.63 | 157.7^{a} |
| 5 | − | oxide-based | High | 21.70^{a} | 13.61 | 0.64 | 27.13 | 161.0^{a} |
| 6 | − | organic-based | Low | 21.85^{a} | 15.40 | 0.94 | 36.21 | 188.1^{a} |
| 7 | + | — | — | 22.81^{a} | 15.35 | 0.79 | 29.67 | 22.9^{b} |
| 8 | + | sulphate-based | Low | 21.06^{a} | 13.64 | 0.61 | 38.22 | 172.2^{a} |
| 9 | + | sulphate-based | High | 21.51^{a} | 14.25 | 0.84 | 32.48 | 183.7^{a} |
| 10 | + | oxide-based | Low | 21.16^{a} | 14.56 | 0.69 | 34.93 | 149.8^{a} |
| 11 | + | oxide-based | High | 22.65^{a} | 14.31 | 0.85 | 26.14 | 189.5^{a} |
| 12 | + | organic | Low | 20.38^{ab} | 14.26 | 0.70 | 34.67 | 146.5^{a} |

Main effects

| | | | | | | | | |
|---|---|---|---|---|---|---|---|---|
| Phytase | − | | | 20.49 | 14.51 | 0.87 | 37.04 | 140.7 |
| | + | | | 21.60 | 14.40 | 0.75 | 32.68 | 144.1 |
| Trace minerals | | — | | 19.88 | 15.18 | 1.01 | 38.70 | 20.1 |
| | | sulphate-based | Low | 20.96 | 14.48 | 0.84 | 36.60 | 172.1 |
| | | sulphate-based | High | 21.29 | 14.10 | 0.77 | 35.01 | 165.9 |
| | | oxide-based | Low | 20.85 | 14.16 | 0.67 | 36.78 | 153.7 |
| | | oxide-based | High | 22.18 | 13.96 | 0.74 | 26.64 | 175.2 |
| | | organic | Low | 21.11 | 14.83 | 0.82 | 35.44 | 167.3 |

| | | | | | |
|---|---|---|---|---|---|
| SEM | 0.757 | 0.741 | 0.152 | 10.554 | 12.51 |
| P-value, phytase | 0.014 | 0.797 | 0.157 | 0.477 | 0.640 |
| P-value trace minerals | 0.101 | 0.543 | 0.343 | 0.895 | <0.001 |
| P-value phytase × trace minerals | 0.002 | 0.430 | 0.104 | 0.942 | 0.037 |

^{a,b,c,d}Means within each column grouping with uncommon superscripts are significantly different at P < 0.05.
¹Treatments 1-6 based on CON1, treatments 7-12 based on CON2 (see Table 1).
²Included at 2,000, 1,500 and 1,000 FTU/kg in starter (d 1-10), grower (d 10-20) and finisher (d 20-35) phases, respectively.
³The levels of individual trace minerals added to each treatment diet, and their sources, are given in Table 1.

Tibia trace mineral content at 10 d of age: The results relating to the effect of trace minerals (TM) supplementation, phytase supplementation, or both, on TM content at 10 d of age are shown in Table 14. There were interactions between TM and phytase for tibia concentrations of Zn and Mn, and a tendency towards an interaction for tibia Fe. Added TM markedly increased tibia Zn (by 64 to 117% in treatments 2-6 vs 1), with greater response levels in TM ‘high’ than TM ‘low’ treatments and in oxide-vs sulphate- or organic-based TM treatments. Added phytase without added TM also markedly increased tibia Zn compared to either no added phytase or TM (treatment 1, +138%) or to added TM but no phytase (treatments 2-6, +10 to +46%). Addition of TM on top of phytase did not further increase tibia Zn.

Tibia Mn was not increased by TM supplementation (without phytase) except in treatment 5 (oxide-based ‘high’). Phytase markedly increased tibia Mn (+29% in treatment 7 vs 1) and added TM on top of phytase further improved tibia Mn, regardless of TM source or dose-level (+24 to +44% in treatments 8-12 vs 7).

Across phytase treatments, tibia Fe was moderately (−4.4 to −7.2%) reduced by supplemental TM in all but one TM treatment (sulphate-based ‘low’ TM). Tibia Fe content was increased by phytase (+7% vs no phytase, across TM treatments).

TABLE 14
Effect of supplementation with trace minerals (low or high) or phytase or both,
on tibia trace mineral content at 10 d of age in Example 1; 2-way ANOVA

| | | Trace mineral | | Zn, | Fe, | Cu, | Mn, |
|---|---|---|---|---|---|---|---|
| Treatment | Phytase | supplementation³ | | mg/kg | mg/kg | mg/kg | mg/kg |
| no.¹ | (+/−)² | Source | Level | DM | DM | DM | DM |

At 10 d of age
Treatment means

| | | | | | | | |
|---|---|---|---|---|---|---|---|
| 1 | − | — | — | 165^{f} | 249 | 4.82 | 4.79^{fg} |
| 2 | − | sulphate-based | Low | 270^{e} | 233 | 3.76 | 5.00^{cfg} |
| 3 | − | sulphate-based | High | 324^{c} | 219 | 2.91 | 5.66^{def} |
| 4 | − | oxide-based | Low | 304^{d} | 224 | 3.47 | 4.86^{g} |
| 5 | − | oxide-based | High | 358^{b} | 230 | 3.88 | 5.72^{de} |
| 6 | − | organic-based | Low | 278^{e} | 224 | 3.56 | 4.72^{g} |
| 7 | + | — | — | 393^{a} | 248 | 3.56 | 6.16^{d} |
| 8 | + | sulphate-based | Low | 397^{a} | 249 | 2.67 | 8.09^{abc} |
| 9 | + | sulphate-based | High | 399^{a} | 246 | 3.54 | 8.84^{a} |
| 10 | + | oxide-based | Low | 394^{a} | 250 | 2.94 | 7.84^{bc} |
| 11 | + | oxide-based | High | 404^{a} | 245 | 3.27 | 8.54^{ab} |
| 12 | + | organic-based | Low | 396^{a} | 237 | 3.16 | 7.62^{c} |

Main effects

| | | | | | | | |
|---|---|---|---|---|---|---|---|
| Phytase | − | | | 283 | 230^{b} | 3.73 | 5.12^{a} |
| | + | | | 397 | 246^{a} | 3.19 | 7.85^{b} |
| Trace minerals | | | | 279 | 249^{a} | 4.19 | 5.47 |
| | | sulphate-based | Low | 334 | 241^{ab} | 3.22 | 6.54 |
| | | sulphate-based | High | 362 | 233^{bc} | 3.23 | 7.25 |
| | | oxide-based | Low | 348 | 237^{bc} | 3.21 | 6.35 |
| | | oxide-based | High | 381 | 238^{bc} | 3.58 | 7.13 |
| | | organic-based | Low | 337 | 231^{c} | 3.36 | 6.17 |

| | | | | |
|---|---|---|---|---|
| SEM | 6.23 | 5.00 | 0.529 | 0.290 |
| P-value, phytase | <0.001 | <0.001 | 0.079 | <0.001 |
| P-value trace minerals | <0.001 | 0.008 | 0.388 | <0.001 |
| P-value phytase × trace minerals | <0.001 | 0.080 | 0.568 | 0.039 |

^{a,b,c,d}Means within each column grouping with uncommon superscripts are significantly different at P < 0.05.
¹Treatments 1-6 were based on CON1, treatments 7-12 were based on CON2 (see Table 2 of the patent application for full composition).
²Included at 2,000, 1,500 and 1,000 FTU/kg in starter (d 0-10), grower (d 10-20) and finisher (d 20-35) phases, respectively.
³The levels of individual trace minerals added to each treatment diet, and their sources, are given in Table 1 in the patent application.

Trace minerals in the liver at 10 d of age: The results relating to the effect of TM supplementation, phytase supplementation, or both, on liver weight and concentrations of trace minerals are shown in Table 15. There was an interaction effect on liver concentrations of Zn: Added TM without phytase had no effect on liver Zn compared with no added TM or phytase, whereas added phytase without added TM increased liver Zn (by 17% in treatment 7 vs 1). Trace mineral supplementation on top of phytase did not lead to further increase in liver Zn (treatments 8-12 vs 7), regardless of TM source or dose.

Liver Fe was increased by phytase (+42% vs no phytase) but not by TM supplementation, although liver Fe was higher in sulphate-based ‘high’ than oxide- or organic-based ‘low’ treatments. Liver Cu concentrations were unaffected by supplemental TM but were reduced by supplemental phytase (2.48 vs 2.57 mg/kg or −3.5% vs no phytase), which could be related to the increased Zn absorption in the phytase-supplemented treatments which may due to competition on absorption transporter (liver Zn was 17.4 with phytase vs 16.6 mg/kg without, +4.8%). Supplemental phytase increased liver Mn (+19% vs no phytase) and, to a lesser degree and only in certain treatments, supplemental TM also increased liver Mn (+7.4 to +18% vs no added TM).

TABLE 15
Effect of supplementation with trace minerals (low or high) or phytase or both,
on liver trace mineral content at 10 d of age in Example 1; 2-way ANOVA

| | | Trace mineral | | Liver | | | | |
|---|---|---|---|---|---|---|---|---|
| Treatment | Phytase | supplementation³ | | weight, | Zn, | Fe, | Cu, | Mn, |
| no.¹ | (+/−)² | Source | Level | % BW | mg/kg | mg/kg | mg/kg | mg/kg |

At 10 d of age:
Treatment means

| | | | | | | | | |
|---|---|---|---|---|---|---|---|---|
| 1 | − | — | — | 3.96 | 15.6^{c} | 57.5 | 2.49 | 1.60 |
| 2 | − | sulphate-based | Low | 3.83 | 16.3^{bc} | 65.0 | 2.54 | 1.85 |
| 3 | − | sulphate-based | High | 3.86 | 17.1^{abc} | 77.5 | 2.60 | 1.90 |
| 4 | − | oxide-based | Low | 3.97 | 16.8^{abc} | 56.9 | 2.65 | 1.77 |
| 5 | − | oxide-based | High | 3.93 | 17.3^{abc} | 62.9 | 2.54 | 1.88 |
| 6 | − | organic-based | Low | 3.95 | 16.6^{abc} | 48.9 | 2.61 | 1.64 |
| 7 | + | — | — | 3.86 | 18.3^{a} | 86.0 | 2.53 | 1.91 |
| 8 | + | sulphate-based | Low | 4.00 | 17.4^{ab} | 92.0 | 2.50 | 2.14 |
| 9 | + | sulphate-based | High | 4.00 | 17.1^{abc} | 93.5 | 2.50 | 2.24 |
| 10 | + | oxide-based | Low | 3.96 | 17.0^{abc} | 70.7 | 2.41 | 2.03 |
| 11 | + | oxide-based | High | 4.05 | 17.4^{ab} | 96.9 | 2.49 | 2.23 |
| 12 | + | organic | Low | 3.93 | 17.3^{abc} | 84.1 | 2.48 | 2.15 |

Main effects

| | | | | | | | | |
|---|---|---|---|---|---|---|---|---|
| Phytase | − | | | 3.92 | 16.6 | 61.4^{b} | 2.57^{a} | 1.77^{a} |
| | + | | | 3.97 | 17.4 | 87.2^{a} | 2.48^{b} | 2.11^{b} |
| Trace minerals | | — | | 3.91 | 16.9 | 71.8^{ab} | 2.51 | 1.76^{b} |
| | | sulphate-based | Low | 3.92 | 16.8 | 78.5^{ab} | 2.52 | 1.99^{a} |
| | | sulphate-based | High | 3.93 | 17.1 | 85.5^{a} | 2.55 | 2.07^{a} |
| | | oxide-based | Low | 3.97 | 16.9 | 63.8^{b} | 2.53 | 1.90^{ab} |
| | | oxide-based | High | 3.99 | 17.3 | 79.9^{ab} | 2.51 | 2.05^{a} |
| | | organic | Low | 3.94 | 16.9 | 66.5^{b} | 2.54 | 1.89^{ab} |

| | | | | | |
|---|---|---|---|---|---|
| SEM | 0.10 | 0.37 | 6.40 | 0.07 | 0.06 |
| P-value, phytase | 0.389 | <0.001 | <0.001 | 0.023 | <0.001 |
| P-value trace minerals | 0.964 | 0.767 | 0.007 | 0.981 | <0.001 |
| P-value phytase × trace minerals | 0.725 | 0.005 | 0.432 | 0.398 | 0.403 |

^{a,b,c,d}Means within each column grouping with uncommon superscripts are significantly different at P < 0.05.
¹Treatments 1-6 based on CON1, treatments 7-12 based on CON2 (see Table 2 of the patent application for full composition).
²Included at 2,000, 1,500 and 1,000 FTU/kg in starter (d 0-10), grower (d 10-20) and finisher (d 20-35) phases, respectively.
³The levels of individual trace minerals added to each treatment diet, and their sources, are given in Table 1 of the patent application.

Conclusion: PhyG phytase supplemented to a corn-soybean meal-based diet, that was reduced in Ca and digestible P and contained a normal level of dietary Ca, totally replaced supplemental TM (Zn, Fe, Cu, Mn and Se) during all growth phases and overall (1 to 35 d of age); growth performance, liver and plasma TM contents were all maintained at levels equivalent to those produced by a nutritionally adequate, control diet supplemented with TM but no phytase. These results indicate that PhyG phytase could be used to replace TM supplementation in monogastric animal diets.

### Example 2: Efficacy of a Novel Consensus Bacterial 6-Phytase Variant to Replace Added Trace Minerals in a Corn-Soybean Meal-Based Diet Containing a High Level of Calcium

This study was carried out at the same research institution as the study in Example 1 (Schothorst Feed Research, Lelystad, the Netherlands) using the same experimental design, treatments, conditions, methods and statistical analysis procedures, except that a higher level of calcium (Ca) was used in the basal diet, as can be encountered in some commercial diets (Petricevic et al. 2002). The ingredient and calculated nutrient composition of the basal diets is presented in Table 10 (formulated Ca in the CON1 diet was 1.14% during starter phase, 1-10 d of age, and 1.06% during grower phase, 10-20 d of age). This compares with the NRC (1994) recommendations of 1.0%, during starter and grower phase, respectively). The concentrations of the individual TM added to each treatment are as in Table 1, whilst the analyzed mineral and TM content of the diets is given in Table 11.

TABLE 10
Ingredient and calculated nutrient composition
of the basal diets in Example 2

| | CON1¹ | | CON2² | |
|---|---|---|---|---|
| | Starter | Grower | Starter | Grower |
| Item | (1-10 d) | (10-20 d) | (1-10 d) | (10-20 d) |

Ingredients, % as fed

| | | | | |
|---|---|---|---|---|
| Corn | 57.0 | 56.7 | 57.0 | 56.7 |
| Soybean meal | 28.3 | 29.1 | 28.3 | 29.1 |
| Rice bran | 2.86 | 2.94 | 2.86 | 2.94 |
| Corn gluten meal | 3.42 | 2.00 | 3.42 | 2.00 |
| Soybean oil | 1.00 | 3.28 | 1.00 | 3.28 |
| Limestone | 2.15 | 2.03 | 1.96 | 1.84 |
| Monocalcium phosphate | 1.61 | 1.40 | 0.61 | 0.44 |
| Silicate | 0.00 | 0.00 | 1.19 | 1.14 |
| Lard | 1.19 | 0.28 | 1.19 | 0.28 |
| Salt | 0.33 | 0.34 | 0.33 | 0.34 |
| L-lysine HCl | 0.42 | 0.26 | 0.42 | 0.26 |
| DL-methionine | 0.31 | 0.26 | 0.31 | 0.26 |
| L-threonine | 0.11 | 0.09 | 0.11 | 0.09 |
| L-tryptophan | 0.03 | 0.01 | 0.03 | 0.01 |
| L-arginine | 0.13 | — | — | — |
| L-valine | 0.07 | 0.00 | 0.07 | 0.00 |
| Titanium dioxide | — | 0.35 | — | 0.35 |
| Vitamin premix³ | 0.50 | 0.50 | 0.50 | 0.50 |
| Test premix (corn | 0.50 | 0.50 | 0.50 | 0.50 |
| only in control) | | | | |
| Sacox | 0.01 | 0.01 | 0.01 | 0.01 |
| Phytase, FTU/kg | — | — | 2,000 | 1,500 |
| Total | 100 | 100 | 100 | 100 |

Chemical composition, % as is (unless otherwise stated)

| | | | | |
|---|---|---|---|---|
| ME, kcal/kg | 2,900 | 2,975 | 2,900 | 2,975 |
| Moisture | 11.7 | 11.6 | 11.7 | 11.6 |
| Ash | 6.2 | 6.2 | 6.2 | 6.2 |
| Crude protein | 21.1 | 20.1 | 21.1 | 20.1 |
| Crude fat | 5.5 | 6.8 | 5.5 | 6.8 |
| Crude fiber | 2.4 | 2.4 | 2.4 | 2.4 |
| Calcium | 1.14 | 1.06 | 0.92 | 0.85 |
| Phosphorus (P) | 0.72 | 0.68 | 0.50 | 0.47 |
| Retainable phosphorus | 0.43 | 0.39 | 0.24 | 0.21 |
| Phytate-P | 0.25 | 0.25 | 0.25 | 0.25 |
| Zinc, mg/kg | 28 | 27 | 27 | 27 |
| Iron, mg/kg | 147 | 137 | 103 | 94 |
| Copper, mg/kg | 5.4 | 5.4 | 5.4 | 5.3 |
| Manganese, mg/kg | 28 | 27 | 23 | 22 |
| Selenium, mg/kg | 0.1 | 0.1 | 0.1 | 0.1 |

¹Formulated to meet the nutrient requirements for broilers applicable in the Netherlands (CVB, 2021) but without added TM
²Reduced in Ca and dig P vs. CON1 (to account for the expected Ca and dig P contribution of the added phytases), without trace mineral supplementation, with addition of phytase at 2,000, 1,500 and 1,000 FTU/kg in starter, grower and finisher phases respectively.
³Supplied per kilogram of diet: vitamin A (retinyl acetate 3a672a, 10,000 IU), vitamin D3 (3a671, 2,500 IU), vitamin E (dl-α-tocopherol 3a700, 50 IU), vitamin K3 (menadione 3a711, 1.5 mg), vitamin B1 (thiamine mononitrate 3a821, 2.0 mg), riboflavin (7.5 mg), D-pantothenic acid (12 mg), vitamin B6 (3.5 mg), vitamin B12 (0.020 mg), niacin (35 mg), folic acid (1.0 mg), biotin (0.20 mg), choline (choline chloride, 460 mg) and I (KI, 0.8 mg).

TABLE 11
Analyzed trace minerals content of the experimental diets in Example 2

| | | Treatment No | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Ca, g/kg | Starter | 11.9 | — | — | — | — | — | 9.68 | — | — | — | — | — |
| | Grower | 10.9 | — | — | — | — | — | 9.38 | — | — | — | — | — |
| P, g/kg | Starter | 7.27 | — | — | — | — | — | 5.02 | — | — | — | — | — |
| | Grower | 6.87 | — | — | — | — | — | 4.72 | — | — | — | — | — |
| Zn, mg/kg | Starter | 25 | 35 | 45 | 40 | 54 | 35 | 25 | 33 | 43 | 41 | 54 | 37 |
| | Grower | 25 | 33 | 43 | 39 | 54 | 38 | 25 | 33 | 44 | 40 | 53 | 33 |
| Fe, mg/kg | Starter | 153 | 160 | 182 | 164 | 183 | 174 | 124 | 120 | 160 | 127 | 135 | 122 |
| | Grower | 148 | 163 | 175 | 162 | 187 | 152 | 102 | 109 | 131 | 115 | 129 | 111 |
| Cu, mg/kg | Starter | 6 | 8 | 12 | 8 | 11 | 8 | 5 | 9 | 11 | 9 | 11 | 10 |
| | Grower | <5 | 7 | 11 | 7 | 10 | 7 | <5 | 7 | 9 | 7 | 10 | 7 |
| Mn, mg/kg | Starter | 30 | 40 | 50 | 39 | 51 | 40 | 24 | 32 | 43 | 36 | 37 | 35 |
| | Grower | 31 | 39 | 47 | 35 | 51 | 37 | 23 | 32 | 42 | 44 | 46 | 33 |
| Se, mg/kg | Starter | 0.11 | 0.23 | 0.24 | 0.23 | 0.28 | 0.27 | 0.10 | 0.23 | 0.23 | 0.25 | 0.28 | 0.22 |
| | Grower | <0.1 | 0.23 | 0.24 | 0.28 | 0.21 | 0.34 | 0.11 | 0.23 | 0.24 | 0.20 | 0.32 | 0.22 |

²Included at 2,000, 1,500 FTU/kg in starter (d 1-10), grower (d 10-20) phases, respectively.
³The levels of individual trace minerals added to each treatment diet, and their sources, are given in Table 1.
⁴Starter: 1 to 10 d of age; Grower: 10 to 20 d of age.

### Results

Growth performance: The results for BW, BWG, FI and FCR by phase are presented in Tables 12-13. During starter phase (1-10 d of age; Table 12), PhyG phytase supplemented to CON2 (treatment 7) improved the growth performance (BWG and FI) of birds to levels that were above those achieved by CON1 treatments supplemented with ‘low’ TM (regardless of TM source; treatments 2, 4 and 6), equivalent to the growth performance responses of birds fed CON1 treatments supplemented with ‘high’ TM (treatments 3 and 5). By 10 d of age, the BW of birds fed CON2 supplemented with phytase but no added TM (treatment 7) was above that of birds fed any of the TM-supplemented CON1 diets, regardless of TM dose level. Without phytase (treatments 2-6), TM mineral supplementation of the CON1 diet improved all measures of growth performance (increased BW, BWG, FI and reduced FCR) during starter phase. This effect was evident across all TM sources and dose levels but there was also evidence of a dose-response effect on BWG and d 10 BW. However, with added phytase (treatments 7-12), there was no dose-response effect; TM supplementation did not provide any further benefit above that achieved by supplemental phytase alone, for any growth performance measure, regardless of TM source or dose level.

TABLE 12
Effect of supplementation with trace minerals (low or high) or phytase
or both, on growth performance during 1-10 d; 2-way ANOVA

| | | Trace mineral | | | | | |
|---|---|---|---|---|---|---|---|
| Treatment | Phytase | supplementation³ | | d 10 BW, | BWG, | FI, | FCR, |
| no.¹ | (+/−)² | Source | Level | g/bird | g/bird | g/bird | g:g |

Treatment means

| | | | | | | | |
|---|---|---|---|---|---|---|---|
| 1 | − | — | — | 256^{e} | 213^{e} | 218^{d} | 1.026 |
| 2 | − | sulphate-based | Low | 305^{d} | 262^{d} | 263^{c} | 1.006 |
| 3 | − | sulphate-based | High | 324^{bc} | 281^{bc} | 282^{ab} | 1.003 |
| 4 | − | oxide-based | Low | 317^{cd} | 274^{cd} | 274^{bc} | 1.001 |
| 5 | − | oxide-based | High | 326^{bc} | 283^{abc} | 285^{ab} | 1.005 |
| 6 | − | organic-based | Low | 307^{d} | 264^{d} | 265^{c} | 1.007 |
| 7 | + | — | — | 341^{a} | 297^{a} | 294^{a} | 0.989 |
| 8 | + | sulphate-based | Low | 335^{ab} | 292^{ab} | 287^{ab} | 0.984 |
| 9 | + | sulphate-based | High | 336^{ab} | 293^{ab} | 288^{ab} | 0.984 |
| 10 | + | oxide-based | Low | 335^{ab} | 292^{ab} | 287^{ab} | 0.982 |
| 11 | + | oxide-based | High | 334^{ab} | 292^{b} | 286^{ab} | 0.981 |
| 12 | + | organic | Low | 332^{abc} | 288^{abc} | 284^{ab} | 0.983 |

Main effects

| | | | | | | | |
|---|---|---|---|---|---|---|---|
| Phytase | − | | | 306 | 263 | 264 | 1.008^{a} |
| | + | | | 335 | 292 | 287 | 0.984^{b} |
| Trace minerals | | — | | 298 | 253 | 254 | 1.008^{a} |
| | | sulphate-based | Low | 320 | 277 | 275 | 0.995^{b} |
| | | sulphate-based | High | 329 | 286 | 284 | 0.994^{b} |
| | | oxide-based | Low | 326 | 283 | 280 | 0.992^{b} |
| | | oxide-based | High | 330 | 287 | 285 | 0.993^{b} |
| | | organic | Low | 319 | 276 | 275 | 0.995^{b} |

| | | | | |
|---|---|---|---|---|
| SEM | 3.115 | 3.158 | 3.059 | 0.004 |
| P-value, phytase | <0.001 | <0.001 | <0.001 | <0.001 |
| P-value trace minerals | <0.001 | <0.001 | <0.001 | <0.001 |
| P-value phytase × trace minerals | <0.001 | <0.001 | <0.001 | 0.123 |

^{a,b,c,d}Means within each column grouping with uncommon superscripts are significantly different at P < 0.05.
¹Treatments 1-6 were based on CON1, treatments 7-12 were based on CON2 (see Table 2).
²Included at 2,000, 1,500 FTU/kg in starter (d 1-10), grower (d 10-20) phases, respectively.
³The levels of individual trace minerals added to each treatment diet, and their sources, are given in Table 1.

During grower phase (10-20 d of age; Table 13), the phytase without added TM (treatment 7) maintained growth performance (BWG, FI, FCR) equivalent to, but not higher than, all treatments containing added TM without added phytase (treatments 2-6). However, due to the extra beneficial effect of the phytase above that of supplemental TM that occurred during starter phase, final BW at 20 d of age was higher in treatment 7 than in some of the individual TM-supplemented treatments (higher than treatment 2, sulphate-based ‘low’ TM and treatment 6, organically-complexed ‘low’ TM). As seen during starter phase, supplemental TM without added phytase during grower phase improved some measures of growth performance (BWG and FI) vs. no added phytase or TM (treatment 1) but feed efficiency (FCR) was not improved by supplemental TM during grower phase. This suggests that the TM-mediated improvements in BWG during grower phase were driven largely by increased FI rather than increased TM availability per se. With phytase, TM supplementation provided no further benefit to growth performance, consistent with the data generated by Example 1.

Mortality was low (<2.8%, on average) during both phases, and unaffected by phytase or TM supplementation.

TABLE 13
Effect of supplementation with trace minerals (low or high), phytase
or both, on growth performance during 10-20 d; 2-way ANOVA

| | | Trace mineral | | | | | |
|---|---|---|---|---|---|---|---|
| Treatment | Phytase | supplementation³ | | d 20 BW, | BWG, | FI, | FCR, |
| no.¹ | (+/−)² | Source | Level | g/bird | g/bird | g/bird | g:g |

Treatment means

| | | | | | | | |
|---|---|---|---|---|---|---|---|
| 1 | − | — | — | 827^{e} | 573^{e} | 718^{d} | 1.253 |
| 2 | − | sulphate-based | Low | 970^{d} | 660^{d} | 833^{c} | 1.263 |
| 3 | − | sulphate-based | High | 1,027^{abc} | 699^{abc} | 872^{abc} | 1.247 |
| 4 | − | oxide-based | Low | 1,000^{bcd} | 682^{bcd} | 855^{abc} | 1.253 |
| 5 | − | oxide-based | High | 1,032^{ab} | 705^{abc} | 888^{a} | 1.259 |
| 6 | − | organic-based | Low | 988^{cd} | 681^{cd} | 849^{bc} | 1.246 |
| 7 | + | — | — | 1,048^{a} | 707^{abc} | 885^{ab} | 1.252 |
| 8 | + | sulphate-based | Low | 1,060^{a} | 723^{a} | 891^{a} | 1.233 |
| 9 | + | sulphate-based | High | 1,049^{a} | 713^{a} | 883^{b} | 1.238 |
| 10 | + | oxide-based | Low | 1,047^{a} | 712^{ab} | 883^{ab} | 1.239 |
| 11 | + | oxide-based | High | 1,045^{a} | 711^{abc} | 881^{ab} | 1.239 |
| 12 | + | organic | Low | 1,047^{a} | 716^{a} | 882^{ab} | 1.233 |

Main effects

| | | | | | | | |
|---|---|---|---|---|---|---|---|
| Phytase | − | | | 977 | 669 | 838 | 1.254^{a} |
| | + | | | 1,049 | 714 | 884 | 1.239^{b} |
| Trace minerals | | — | | 938 | 640 | 802 | 1.252 |
| | | sulphate-based | Low | 1,015 | 691 | 862 | 1.248 |
| | | sulphate-based | High | 1,038 | 706 | 877 | 1.242 |
| | | oxide-based | Low | 1,023 | 697 | 869 | 1.246 |
| | | oxide-based | High | 1,039 | 708 | 884 | 1.249 |
| | | organic | Low | 1017 | 698 | 865 | 1.240 |

| | | | | |
|---|---|---|---|---|
| SEM | 8.491 | 6.475 | 8.369 | 0.006 |
| P-value, phytase | <0.001 | <0.001 | <0.001 | <0.001 |
| P-value trace minerals | <0.001 | <0.001 | <0.001 | 0.448 |
| P-value phytase × trace minerals | <0.001 | <0.001 | <0.001 | 0.326 |

^{a,b,c,d}Means within each column grouping with uncommon superscripts are significantly different at P < 0.05.
¹Treatments 1-6 were based on CON1, treatments 7-12 were based on CON2 (see Table 2).
²Included at 2,000, 1,500 FTU/kg in starter (d 1-10), grower (d 10-20) phases, respectively.
³The levels of individual trace minerals added to each treatment diet, and their sources, are given in Table 1.

Overall Conclusions: In a corn-soybean meal-based diet containing a normal level of calcium (Example 1), PhyG phytase added to diets in a tiered dosing regimen by phase totally replaced supplemental TM during all growth phases and overall, regardless of TM source or dose level. The same phytase also totally replaced supplemental TM during starter and grower phase when added to the same corn-soybean meal-based diet containing a high level of calcium (1.19% and 1.09% in the diet without added phytase, 0.968% and 0.687% in the diet with added phytase during starter and grower phases, respectively, analyzed values; Example 2). However, in the high-Ca dict there was also evidence of a dose-response effect of the phytase; the phytase added to diets without supplemental TM achieved an average weight gain that was equivalent to that achieved by a ‘high’ dose of supplemental TM and superior to that achieved by a ‘low’ dose of supplemental TM.

### Example 3: Efficacy of a Novel Consensus Bacterial 6-Phytase Variant Used in a Commercial Corn-Soybean Meal-Based Diet to Replace Added Trace Minerals in Young Broilers Raised Under Commercial Settings

This Example evaluated whether a novel consensus bacterial 6-phytase variant (SEQ ID NO: 26) could totally replace added trace minerals supplemented to the diet at a commercial dose level in young broilers during 0 to 21 days of age, when raised under commercial settings.

### Materials and Methods

The study was carried out in accordance with European Directive 2010/63/EU and the regulations in force in the Netherlands for the care and use of animals in research. Experimental protocols and procedures were evaluated and approved by the Ethical Committee on Animal Experiments (Ethische Toetsing Dierproeven) of Schothorst Feed Research (Lelystad, the Netherlands).

Experimental design, birds and housing: The experiment was carried out as a randomized complete block design with four dietary treatments and eight replicate floor pens per treatment. Each replicate pen was an experimental unit containing 820 birds (males and females, as hatched). The stocking density was 17 birds/m². Diets were fed as pelleted diets (pelleting temperature≤80° C.), ad lib, during each of two phases. Temperature and lighting were according to breeder recommendations.

Dietary treatments: Treatment diets were formulated in two phases and focussed on the most sensitive part of the broiler life: 1-10 and 10-21 d of age as starter and grower phases, respectively. Treatments comprised:
1) CON1: control diet, nutritionally adequate but without supplemental Zn, Cu, Fe, Mn;
2) CON1+TM: as CON1 but containing supplemental trace minerals (Zn, Cu, Fe, Mn) from addition of a standard mineral premix, expected to supply 60 mg/kg of Zn, 10 mg/kg of Cu, 50 mg/kg of Fe and 70 mg/kg of Mn in the diet;
3) CON2: CON1 reformulated with reduced Ca and retainable P according to the mineral matrix of the phytase and supplemented with a novel consensus bacterial 6-phytase variant (PhyG) at 2,000 FTU/kg and 1,500 FTU/kg in starter and grower phases, respectively without supplemental Zn, Cu, Fe, Mn;
4) CON2+TM: CON1 reformulated with reduced Ca and retainable P according to the mineral matrix of the phytase, supplemented with PhyG at 2,000 FTU/kg and 1,500 FTU/kg during starter and grower phases, respectively, and additionally supplemented with Zn, Cu, Fe and Mn as in CON1+TM.

The treatment details are given in Table 16. The ingredient and calculated nutrient composition of the diets are given in Table 17.

TABLE 16
Overview of treatments

| | | | Addition of | Phytase (PhyG), | |
|---|---|---|---|---|---|
| Treatment | Treatment | Phytase | Zn, Cu, Fe | FTU/kg | |
| No. | description | (+/−) | and Mn (+/−) | Starter | Grower |
| 1 | CON1 | − | − | — | — |
| 2 | CON1 + TM | − | + | — | — |
| 3¹ | CON2 | + | − | 2,000 | 1,500 |
| 4² | CON2 + TM | + | + | 2,000 | 1,500 |

¹The commercial premix in treatment 2 and 4 was added to supply vitamins and trace minerals including Zn, Cu, Fe and Mn at 60, 10, 50, 70 mg/kg in the diet, respectively. The premix in treatment 1 and 3 provided vitamins and other trace minerals except added Zn, Cu, Fe and Mn (see Table 17)
²In treatments 3 and 4, Ca and retainable P in the diets were reduced by 2.26 and 1.86 g/kg, respectively, in starter phase, and by 2.18 and 1.79 g/kg, respectively, in grower phase.

TABLE 17
Ingredient and calculated nutrient composition of the diets

| | | | | |
|---|---|---|---|---|
| (1-10 d of age) | Trt. 1 | Trt. 2 | Trt. 3 | Trt. 4 |
| Item | CON1 | CON1 + TM | CON2 | CON2 + TM |

Ingredients, % as fed

| | | | | |
|---|---|---|---|---|
| Corn | 54.182 | 54.182 | 54.182 | 54.182 |
| Soybean meal | 30.39 | 30.39 | 30.39 | 30.39 |
| Rapeseed meal | 5.997 | 5.997 | 5.997 | 5.997 |
| Soybean oil | 3.167 | 3.167 | 3.167 | 3.167 |
| Corn gluten meal | 2 | 2 | 2 | 2 |
| Sodium bicarbonate | 0.121 | 0.121 | 0.121 | 0.121 |
| Salt | 0.132 | 0.132 | 0.132 | 0.132 |
| L-lysine HCl | 0.279 | 0.279 | 0.279 | 0.279 |
| DL-methionine | 0.246 | 0.246 | 0.246 | 0.246 |
| L-threonine | 0.044 | 0.044 | 0.044 | 0.044 |
| L-tryptophan | 0.005 | 0.005 | 0.005 | 0.005 |
| Sacox | 0.058 | 0.058 | 0.058 | 0.058 |
| Special vitamin-mineral premix 1 | 1 | — | 1 | — |
| Standard vitamin-mineral premix 2 | — | 1 | — | 1 |
| Limestone | 0.803 | 0.708 | 0.627 | 0.532 |
| Monocalcium phosphate | 1.576 | 1.576 | 0.572 | 0.572 |
| Silica | — | 0.095 | 1.18 | 1.275 |
| Total | 100 | 100 | 100 | 100 |

Calculated nutrients, g/kg (unless otherwise stated)

| Apparent metabolizable energy (kcal/kg) | 2,900 | 2,899 | 2,900 | 2,899 |
|---|---|---|---|---|
| Moisture | 119 | 119 | 119 | 119 |
| Ash | 66.5 | 65.9 | 54.8 | 54.0 |
| Crude protein | 226 | 226 | 226 | 226 |
| Crude fat | 64.4 | 64.4 | 64.4 | 64.4 |
| Crude fiber | 29 | 29 | 29 | 29 |
| Calcium | 8.8 | 8.8 | 6.5 | 6.5 |
| Phosphorus (P) | 7.23 | 7.23 | 5.04 | 5.04 |
| Retainable P | 4.3 | 4.3 | 2.44 | 2.44 |
| Phytate-P | 2.46 | 2.46 | 2.46 | 2.46 |
| Manganese, mg/kg | 43.4 | 75.2 | 37.1 | 68.9 |
| Selenium, mg/kg | 0.24 | 0.44 | 0.24 | 0.44 |
| Zinc, mg/kg | 34.8 | 68.7 | 33.6 | 67.5 |
| Copper, mg/kg | 5.11 | 13.63 | 5.14 | 13.66 |
| Iron, mg/kg | 326 | 349 | 277 | 300 |
| SID lysine | 12.1 | 12.1 | 12.1 | 12.1 |
| SID methionine | 5.68 | 5.68 | 5.68 | 5.68 |
| SID cysteine | 2.91 | 2.91 | 2.91 | 2.91 |
| SID methionine + cysteine | 8.59 | 8.59 | 8.59 | 8.59 |
| SID threonine | 7.38 | 7.38 | 7.38 | 7.38 |
| SID tryptophan | 2.18 | 2.18 | 2.18 | 2.18 |
| SID valine | 9.09 | 9.09 | 9.09 | 9.09 |
| (10-21 d of age) | Tr. 1 | Trt. 2 | Trt. 3 | Trt. 4 |
| Item | CON1 | CON1 + TM | CON2 | CON2 + TM |

Ingredients, % as fed

| | | | | |
|---|---|---|---|---|
| Corn | 57.657 | 57.657 | 57.657 | 57.657 |
| Soybean meal | 26.913 | 26.913 | 26.913 | 26.913 |
| Rapeseed meal | 6 | 6 | 6 | 6 |
| Soybean oil | 3.562 | 3.562 | 3.562 | 3.562 |
| Corn gluten meal | 2 | 2 | 2 | 2 |
| Sodium bicarbonate | 0.102 | 0.102 | 0.102 | 0.102 |
| Salt | 0.147 | 0.147 | 0.147 | 0.147 |
| L-lysine HCl | 0.228 | 0.228 | 0.228 | 0.228 |
| DL-methionine | 0.198 | 0.198 | 0.198 | 0.198 |
| L-threonine | 0.045 | 0.045 | 0.045 | 0.045 |
| Sacox | 0.058 | 0.058 | 0.058 | 0.058 |
| Special vitamin-mineral premix 1 | 1 | — | 1 | — |
| Standard vitamin-mineral premix 2 | — | 1 | — | 1 |
| Limestone | 0.697 | 0.602 | 0.531 | 0.435 |
| Monocalcium phosphate | 1.393 | 1.393 | 0.43 | 0.43 |
| Silica | — | 0.095 | 1.129 | 1.225 |
| Total | 100 | 100 | 100 | 100 |

Calculated nutrients, g/kg (unless otherwise stated)

| | | | | |
|---|---|---|---|---|
| Apparent metabolizable energy (kcal/kg) | 2,975 | 2,974 | 2,975 | 2,974 |
| Moisture | 119 | 119 | 119 | 119 |
| Ash | 61.7 | 61 | 50.5 | 49.8 |
| Crude protein | 212 | 212 | 212 | 212 |
| Crude fat | 69.2 | 69.2 | 69.2 | 69.2 |
| Crude fiber | 28.4 | 28.4 | 28.4 | 28.4 |
| Calcium | 8 | 8 | 5.8 | 5.8 |
| Phosphorus (P) | 6.71 | 6.71 | 4.61 | 4.61 |
| Retainable P | 3.9 | 3.9 | 2.11 | 2.11 |
| Phytate-P | 2.43 | 2.43 | 2.43 | 2.43 |
| Manganese, mg/kg | 40.7 | 72.5 | 34.6 | 66.4 |
| Selenium, mg/kg | 0.23 | 0.43 | 0.23 | 0.43 |
| Zinc, mg/kg | 34 | 67.9 | 32.9 | 66.8 |
| Copper, mg/kg | 4.79 | 13.31 | 4.81 | 13.33 |
| Iron, mg/kg | 298 | 321 | 251 | 274 |
| SID lysine | 10.9 | 10.9 | 10.9 | 10.9 |
| SID methionine | 5.05 | 5.05 | 5.05 | 5.05 |
| SID cysteine | 2.79 | 2.79 | 2.79 | 2.79 |
| SID methionine + cysteine | 7.85 | 7.85 | 7.85 | 7.85 |
| SID threonine | 6.98 | 6.98 | 6.98 | 6.98 |
| SID tryptophan | 1.96 | 1.96 | 1.96 | 1.96 |
| SID valine | 8.55 | 8.55 | 8.55 | 8.55 |

¹The special vitamin-mineral premix had the same chemical composition as the standard vitamin-mineral premix except it did not include Zn, Cu, Fe and Mn.
²The standard vitamin-mineral premix was a commercial premix that provided per kilogram of feed: 9000 IU vitamin A, 2800 IU vitamin D3; 20 IU vitamin E; 2 mg vitamin K₃; 2 mg vitamin B1; 5 mg vitamin B₂; 10.9 mg Ca-D-pantothenic acid; 40 mg niacinamide; 3 mg vitamin B₆; 1 mg folic acid; 20 μg vitamin B₁₂; 70 μg biotin; 197 mg betaine hydrochloride; 50 mg Fe from sulphate; 1 mg iodine, 10 mg Cu from sulphate; 70 mg Mn from oxide; 60 mg Zn from oxide; 0.2 mg Se from Na-Selenium
SID, standardized ileal digestible.

TABLE 18
Calculated and (in italics) analyzed macrominerals (Ca and P; g/kg) and microminerals
TM: Fe, Cu, Mn, Zn; mg/kg) in the experimental diets of Example 3.

| | | Trace minerals | | | | | | |
|---|---|---|---|---|---|---|---|---|
| | Phytase | (Zn, Cu, Fe, Mn) | | | | | | |
| Treatment | (+/−)² | (+/−) | Ca | P | Fe | Cu | Mn | Zn |

Starter

| | | | | | | | | | | | | | | |
|---|---|---|---|---|---|---|---|---|---|---|---|---|---|---|
| CON1 | − | − | 8.80 | 8.57 | 7.23 | 7.28 | 326 | 329 | 5 | 8.5 | 43 | 41 | 35 | 34 |
| CON1 + TM | − | + | 8.80 | 8.50 | 7.23 | 7.77 | 349 | 381 | 14 | 16.5 | 75 | 91 | 69 | 96 |
| CON2 | + | − | 6.50 | 6.89 | 5.04 | 5.34 | 277 | 300 | 5 | 6.6 | 37 | 39 | 34 | 39 |
| CON2 + TM | + | + | 6.50 | 6.60 | 5.04 | 5.43 | 300 | 312 | 14 | 13.5 | 69 | 67 | 68 | 76 |

Grower

| | | | | | | | | | | | | | | |
|---|---|---|---|---|---|---|---|---|---|---|---|---|---|---|
| CON1 | − | − | 8.00 | 7.68 | 6.71 | 7.01 | 298 | 278 | 5 | 5.0 | 41 | 37 | 34 | 34 |
| CON1 + TM | − | + | 8.00 | 7.88 | 6.71 | 6.67 | 321 | 352 | 13 | 12.5 | 73 | 84 | 68 | 87 |
| CON2 | + | − | 5.80 | 6.02 | 4.61 | 4.76 | 251 | 242 | 5 | 5.0 | 35 | 31 | 33 | 34 |
| CON2 + TM | + | + | 5.80 | 5.90 | 4.61 | 4.64 | 274 | 279 | 13 | 13.5 | 66 | 66 | 67 | 74 |

²Included at 2,000, 1,500 FTU/kg in starter and grower phases, respectively.
⁴Starter (1 to 10 d of age); grower (10 to 21 d of age).

Sampling, measurements and statistical analysis: Body weight was determined on a per pen basis at the study start and end of each phase and used to calculated BWG. Feed disappearance was measured per phase to determine FI. Feed conversion ratio was calculated per pen per phase and corrected for mortality. At 20 d of age, two male and two female birds per pen were randomly selected and euthanized with CO₂. The left and right tibias were collected, pooled per pen, and stored at −20° C. For tibia ash analysis, the left tibias were thawed, autoclaved, and cleaned. The cleaned tibias were oven dried using standard procedures and the bones defatted using 100% petroleum ether according to a Soxhlet principle. Defatted pooled tibias were air-dried, oven-dried and then incinerated in a muffle furnace, first at 500° C. and then at 700° C. for 18 h. Trace minerals were analyzed by inductively coupled plasma-optical emission spectrometry (ICP-OES) in duplicate.

Outlier analysis was performed prior to data analysis. Outliers were defined as values with residuals exceeding 2.5 times the standard error of the mean. If any one of the growth performance response measures per phase was identified as an outlier, all performance response measures were removed from the dataset for that phase. Data were analyzed by one-way ANOVA. Pen was the experimental unit. Treatment means were separated using Tukey's HSD test. P<0.05 was considered statistically significant. 0.05≤P<0.1 was considered a tendency.

### Results

Growth performance: The results for BW, BWG, FI and FCR by phase and cumulative are presented in Table 19. For the overall period (1 to 21 d of age), phytase without supplemental TM (CON2) increased BW and BWG and reduced FCR compared to no phytase and no supplemental TM (CON1), in each case to a level equivalent to those achieved by supplementation of TM to CON1 (CON1+TM). Adding supplemental TM on top of supplemental phytase did not lead to further increase in BW, BWG or FCR for the overall period. Trace mineral supplementation and phytase supplementation had no effect on FI during the overall period or during any individual phase. Mortality levels were consistently low overall (below 1%) and were unaffected by treatment (data not shown).

TABLE 19
Effect of supplementation with trace minerals or phytase or both,
on growth performance during 1-21 d of age; 1-way ANOVA.

| | | | | | | |
|---|---|---|---|---|---|---|
| | Phytase | TM | BW¹, | BWG, | FI, | FCR, |
| Item | (+/−) | (+/−) | g/bird | g/bird | g/bird | g:g |

Starter, 1 to 10 d of age

| | | | | | | |
|---|---|---|---|---|---|---|
| CON1 | − | − | 268.8^{b} | 228.4^{b} | 268.0 | 1.173 |
| CON1 + TM | − | + | 268.6^{b} | 228.3^{b} | 264.6 | 1.158 |
| CON2 | + | − | 274.9^{a} | 234.5^{a} | 272.3 | 1.161 |
| CON2 + TM | + | + | 269.9^{b} | 229.5^{b} | 269.1 | 1.173 |
| SEM | | | 0.966 | 0.966 | 6.379 | 0.026 |
| P-value | | | <0.001 | <0.001 | 0.727 | 0.926 |

Grower, 10 to 21 d of age

| | | | | | | |
|---|---|---|---|---|---|---|
| CON1 | − | − | 914.4^{b} | 647.1^{b} | 923.2 | 1.427^{a} |
| CON1 + TM | − | + | 924.4^{ab} | 655.8^{ab} | 919.7 | 1.403^{ab} |
| CON2 | + | − | 940.3^{a} | 665.9^{ab} | 916.2 | 1.376^{b} |
| CON2 + TM | + | + | 939.0^{a} | 669.1^{a} | 921.4 | 1.377^{b} |
| SEM | | | 4.977 | 4.906 | 6.341 | 0.011 |
| P-value | | | 0.003 | 0.022 | 0.872 | 0.007 |

0 to 21 d of age

| | | | | | |
|---|---|---|---|---|---|
| CON1 | − | − | 874.05 | 1,191.8 | 1.364^{a} |
| CON1 + TM | − | + | 888.4^{ab} | 1,183.0 | 1.331^{b} |
| CON2 | + | − | 900.2^{a} | 1,191.3 | 1.323^{b} |
| CON2 + TM | + | + | 898.7^{a} | 1,190.5 | 1.325^{b} |
| SEM | | | 4.533 | 4.891 | 0.005 |
| P-value | | | <0.001 | 0.490 | <0.001 |

¹Values presented are for the end of the relevant phase.

Bone mineralization: The effect of supplementation with trace minerals, phytase or both, on the concentrations of trace minerals in tibia bones, is presented in Table 20. The withdrawal of TM supplementation in CON1 reduced Mn and Zn concentrations in bone vs. CON1+TM. Supplemental phytase without supplemental TM in CON2 increased bone Mn and bone Zn vs. CON1 without TM (by 2.61 mg/kg or 47.6% and 61.1 mg/kg or 20.3% respectively). Adding supplemental TM on top of phytase in CON2+TM led to a further increase in tibia Mn (+2.65 mg/kg or 32.8%) but no further increase in tibia Zn, vs. CON2.

TABLE 20
Effect of supplementation with trace minerals or phytase or both,
on bone minerals in broilers at 20 days of age; 1-way ANOVA

| | Phytase | TM | Ca, | P, | Fe, | Cu, | Mn, | Zn, |
|---|---|---|---|---|---|---|---|---|
| Treatment | (+/−) | (+/−) | g/kg | g/kg | mg/kg | mg/kg | mg/kg | mg/kg |
| CON1 | − | − | 372.1 | 190.5 | 243.4^{b} | 2.24 | 5.48^{c} | 301.1^{c} |
| CON1 + TM | − | + | 371.4 | 192.6 | 257.7^{ab} | 2.30 | 7.75^{b} | 339.4^{b} |
| CON2 | + | − | 371.3 | 190.6 | 256.2^{ab} | 2.28 | 8.09^{b} | 362.2^{a} |
| CON2 + TM | + | + | 368.9 | 191.7 | 272.9^{a} | 2.44 | 10.74^{a} | 359.4^{a} |
| SEM | | | 2.028 | 2.683 | 8.238 | 0.215 | 0.257 | 3.723 |
| P-value | | | 0.473 | 0.767 | 0.052 | 0.797 | <0.001 | <0.001 |

Conclusion: PhyG phytase supplemented to a corn-soybean meal-based diet totally replaced commercial levels of supplemental Zn, Fe, Cu and Mn in broilers during 1 to 21 d of age when reared in a commercial setting. The phytase dosed at 2,000 FTU/kg during starter phase and 1,500 FTU/kg during grower phase maintained growth performance (all measures, during starter phase and grower phase and overall) and tibia bone TM concentrations at levels equivalent (in case of Zn concentration it was improved) to those produced by a nutritionally adequate diet supplemented with TM but no phytase. These results indicate that PhyG phytase could be used to replace commercially applied levels of the TM including Zn, Fe, Cu and Mn in the diets of young broilers.

### Example 4: Efficacy of a Novel Consensus Bacterial 6-Phytase Variant Used in a Commercial Corn-Soybean Meal-Based Dict to Replace Added Trace Minerals in Piglets

This Example evaluated if a phytase (PhyG; SEQ ID NO:26) can totally replace trace mineral (TM; Zn, Cu, Fe and Mn) supplemented at commercial dose levels in piglets from 7 to 25 kg BW.

### Materials and Methods

The study was carried out at the University of Applied Sciences Bingen, Germany.

Experimental design, animals and housing: The experiment was carried out as a randomized complete block design with 3 dietary treatments and 12 replicates/treatment, 4 piglets per pen (1:1 males/female). The size of the pen was 3 m×3 m. Lighting program was 16 hours light and 8 hours dark.

In total 144 DanBred x Pi weaning piglets (weaning age 28 days) with an initial BW of 7.0 (+0.44) kg (on day of weaning) were used in this study. Diets were based on corn and SBM and fed in two phases (1-14 days and 14-42 days on trial).

Dietary treatments: Treatments comprised:
1) CON1: control diet, nutritionally adequate but without supplemental Zn, Cu, Fe, Mn
2) CON1+TM: as CON1 containing supplemental trace minerals at a commercial typical level (Zn at 120 ppm, Cu at 80 ppm, Fe at 96 ppm, Mn at 80 ppm)
3) CON2: CON1 reformulated without added inorganic P and reduced Ca, supplementation with a novel consensus phytase (PhyG) at 1500, 1000 FTU/kg in phase 1 and 2, respectively, without supplementation of Zn, Cu, Fe, Mn The treatment details are given in Table 21. The ingredient and calculated nutrient composition of the diets are given in Table 22.

TABLE 21
Overview of treatments

| | | | | Phytase dose | |
|---|---|---|---|---|---|
| | | | Added trace mineral | level (FTU/kg) | |
| Tr | Description | Phytase | (Zn, Cu, Fe, Mn) | Phase 1 | Phase 2 |
| 1 | Con 1 | − | − | — | — |
| 2 | Con 1 + TM¹ | − | + | — | — |
| 3 | CON2² | + | − | 1500 | 1,000 |

¹Supplemented with premix provide: 120 ppm Zn from ZnSO₄, 80 ppm Cu from CuSO₄, 96 ppm Fe from FeSO₄, 80 ppm Mn from MnSO₄
²In treatment 3, Ca was reduced and mono-calcium phosphate (MCP) was excluded.

TABLE 22
Feed formulation and calculated nutrients:

| | T1 | T2 | T3 |
|---|---|---|---|
| | CON1 | CON1 + TM | CON2 |
| Phase 1 | | | |
| Ingredients, % as fed | | | |
| Corn | 40.00 | 40.00 | 40.00 |
| Wheat | 11.10 | 11.10 | 12.00 |
| Barley | 12.00 | 12.00 | 12.00 |
| SBM (46 CP) | 22.00 | 22.00 | 22.00 |
| Rapeseed meal | 3.000 | 3.000 | 3.000 |
| Whey Powder / Lactose | 5.000 | 5.000 | 5.000 |
| Oil/Fat (veg.) | 2.600 | 2.600 | 2.500 |
| L-Lys (HCl) | 0.600 | 0.600 | 0.600 |
| DL-Met | 0.200 | 0.200 | 0.200 |
| L-Thr | 0.200 | 0.200 | 0.200 |
| L-Trp | 0.050 | 0.050 | 0.050 |
| L-Val | 0.050 | 0.050 | 0.050 |
| Sodium Chloride | 0.400 | 0.400 | 0.400 |
| Calcium carbonate | 0.800 | 0.800 | 1.000 |
| MCP | 1.000 | 1.000 | — |
| Standard premix¹ | — | 1.000 | — |
| Premix -without TM² | 1.000 | — | 1.000 |
| PHYTASE | — | — | 1500 FTU/kg |
| | 100.00 | 100.00 | 100.00 |
| Calculated nutrients | | | |
| ME MJ/kg | 13.8 | 13.8 | 13.8 |
| NE MJ/kg | 10.5 | 10.5 | 10.5 |
| Ash, % | 5.83 | 5.83 | 5.03 |
| Crude Protein, % | 18.0 | 18.0 | 18.0 |
| Crude Fat, % | 5.28 | 5.28 | 5.29 |
| Crude Fiber, % | 3.05 | 3.05 | 3.07 |
| Starch, % | 40.1 | 40.1 | 40.6 |
| Lys (SID Lys) % | 1.35 (1.24) | 1.35 (1.24) | 1.35 (1.24) |
| Met (SIDMet), % | 0.48 (0.46) | 0.48 (0.46) | 0.48 (0.46) |
| Met/Cys (SID M + C), % | 0.80 (0.73) | 0.80 (0.73) | 0.80 (0.73) |
| Thr (SID Thr), % | 0.86 (0.76) | 0.86 (0.76) | 0.86 (0.76) |
| Trp, (SID Trp)% | 0.24 (0.22) | 0.24 (0.22) | 0.24 (0.22) |
| Val, (SID Val)% | 0.88 (0.78) | 0.88 (0.78) | 0.88 (0.78) |
| Ca, % | 0.72 | 0.72 | 0.60 |
| P, % | 0.60 | 0.60 | 0.38 |
| ATTD P, % | 0.33 | 0.33 | 0.14 |
| Sodium, % | 0.20 | 0.20 | 0.20 |
| Copper, mg/kg | 5 | 78 | 5 |
| Manganese, mg/kg | 23 | 115 | 22 |
| Zinc, mg/kg | 27 | 132 | 27 |
| Iron, mg/kg | 177 | 263 | 122 |
| Phase 2 | | | |
| Ingredients (%) as fed | | | |
| Corn | 43.00 | 43.00 | 43.00 |
| Wheat | 11.25 | 11.25 | 11.55 |
| Barley | 15.00 | 15.00 | 15.00 |
| SBM (46 CP) | 18.00 | 18.00 | 18.00 |
| Rapeseed meal | 6.000 | 6.000 | 6.000 |
| Oil/Fat (veg.) | 2.900 | 2.900 | 2.900 |
| L-Lys (HCl) | 0.610 | 0.610 | 0.610 |
| DL-Met | 0.170 | 0.170 | 0.170 |
| L-Thr | 0.170 | 0.170 | 0.170 |
| L-Trp | 0.050 | 0.050 | 0.050 |
| L-Val | 0.050 | 0.050 | 0.050 |
| Sodium Chloride | 0.400 | 0.400 | 0.400 |
| Calcium carbonate | 0.900 | 0.900 | 1.100 |
| MCP | 0.500 | 0.500 | — |
| Standard premix¹ | — | 1.000 | — |
| Premix -without TM² | 1.000 | — | 1.000 |
| PHYTASE | — | — | 1000 FTU/kg |
| | 100.00 | 100.00 | 100.00 |
| Calculated nutrients | | | |
| ME MJ/kg | 13.7 | 13.7 | 13.7 |
| NE MJ/kg | 10.4 | 10.4 | 10.4 |
| Ash, % | 5.6 | 5.6 | 5.7 |
| Crude Protein, % | 17.0 | 17.0 | 17.0 |
| Crude Fat, % | 5.8 | 5.8 | 5.7 |
| Crude Fiber, % | 3.3 | 3.3 | 3.3 |
| Starch, % | 43.7 | 43.7 | 44.2 |
| Lys (SID Lys) % | 1.27 (1.15) | 1.27 (1.15) | 1.27 (1.15) |
| Met (SIDMet), % | 0.44 (0.42) | 0.44 (0.42) | 0.45 (0.42) |
| Met/Cys (SID M + C), % | 0.76 (0.69) | 0.76 (0.69) | 0.76 (0.69) |
| Thr (SID Thr), % | 0.78 (0.68) | 0.78 (0.68) | 0.79 (0.68) |
| Trp, (SID Trp)% | 0.22 (0.20) | 0.22 (0.20) | 0.22 (0.20) |
| Val, (dig Val)% | 0.83 (0.72) | 0.83 (0.72) | 0.83 (0.72) |
| Ca, % | 0.72 | 0.72 | 0.61 |
| P, % | 0.60 | 0.60 | 0.37 |
| ATTD P, % | 0.33 | 0.33 | 0.12 |
| Sodium, % | 0.20 | 0.20 | 0.20 |
| Copper, mg/kg | 5 | 78 | 5 |
| Manganese, mg/kg | 24 | 116 | 23 |
| Zinc, mg/kg | 28 | 132 | 27 |
| Iron, mg/kg | 154 | 240 | 127 |

¹The standard vitamin-mineral premix was a commercial premix that provided per kilogram of feed: 16000 IU vitamin A, 2000 IU vitamin D3; 100 mg vitamin E: 4 mg vitamin K₃; 97.5 mg vitamin C; 250 mg vitamin H; 2 mg vitamin B1; 8 mg vitamin B₂; 20 mg Ca-D-pantothenic acid; 30 mg niacinamide; 6 mg vitamin B₆; 2 mg folic acid; 40 mg vitamin B₁₂; 500 mg Choline chloride; 96 mg Fe from sulphate; 2.5 mg iodine, 80 mg Cu from sulphate; 80 mg Mn from sulphate; 120 mg Zn from sulphate; 0.4 mg Se from Na-Selenium
²The special vitamin-mineral premix had the same chemical composition as the standard vitamin-mineral premix except it did not include Zn, Cu, Fe and Mn.

SID, standardized ileal digestible.
The analyzed nutrients and trace mineral content in the diets are given in Table 23.

TABLE 23
Analyzed nutrients (as fed) and trace minerals in the diets:

| | T1 | T2 | T3 |
|---|---|---|---|
| | CON1 | CON1 + TM | CON2 |
| Phase 1 | | | |
| DM, % | 85.8 | 85.7 | 86.7 |
| Ash, % | 5.2 | 5.2 | 5.3 |
| Crude Protein, % | 17.1 | 17.5 | 17.5 |
| Crude Fat, % | 5.2 | 4.5 | 4.4 |
| Crude Fiber, % | 3.4 | 3.4 | 4.3 |
| Ca, % | 0.82 | 0.82 | 0.73 |
| P, % | 0.65 | 0.64 | 0.43 |
| Copper, mg/kg | 5.7 | 58.0 | 5.6 |
| Manganese, mg/kg | 34.0 | 51.0 | 30.0 |
| Zinc, mg/kg | 42.5 | 130.5 | 39.5 |
| Iron, mg/kg | 216 | 284 | 162 |
| Phase 2 | | | |
| DM, % | 84.9 | 86.1 | 84.8 |
| Ash, % | 6.9 | 6.8 | 6.4 |
| Crude Protein, % | 16.5 | 16.7 | 16.4 |
| Crude Fat, % | 4.2 | 4.8 | 4.4 |
| Crude Fiber, % | 3.5 | 3.7 | 3.9 |
| Ca, % | 0.86 | 0.85 | 0.77 |
| P, % | 0.64 | 0.65 | 0.41 |
| Copper, mg/kg | 6.0 | 66.5 | 5.6 |
| Manganese, mg/kg | 32.0 | 53.5 | 29.5 |
| Zinc, mg/kg | 42.0 | 138.5 | 44.0 |
| Iron, mg/kg | 228 | 282 | 168 |

### Sampling, Measurements and Statistical Analysis:

At the end (day 42 on trial) of study, blood samples were collected from 1 piglet per pen for analysis of trace minerals content. Body weight (BW) and feed intake were recorded per phase and at the start and end of each feeding period. Pigs were weighed and recorded individually and the average for each pen was calculated. Feed was weighed and pen consumption was calculated for each phase. Feed conversion ratio (FCR) was calculated based on average pen body weight gain and average pen feed intake and corrected for mortality.

A one-way ANOVA was performed for the performance and blood trace mineral parameters using JMP (version 16.1). Data on the performance parameters including BW and average daily gain (ADG) were analyzed with each pig as experimental unit, using treatment as a fixed factor, initial body weight as covariance, block and sex as random factors. Average daily feed intake (ADFI) and FCR were analyzed using pen as experimental unit, treatment as a fixed factor. Treatment means were separated using Tukey's HSD test. P<0.05 was considered statistically significant. 0.05≤P<0.1 was considered a tendency.

### Results

Growth performance: The results for BW, ADG, ADFI and FCR by phase and cumulative are presented in Table 24.

Phytase without supplemental TM (CON2) increased BW and overall ADG compared to no phytase and no supplemental TM (CON1), in each case to a level equivalent to those achieved by supplementation of TM to CON1 (CON1+TM). Trace mineral supplementation and phytase supplementation had no effect on ADFI or FCR during the overall period or during any individual phase. Mortality was low (<5%) and there was no difference between treatments (data not shown).

TABLE 24
Effect of supplementation with trace minerals or phytase
or both, on growth performance in piglets; 1-way ANOVA.

| | | | BW, | BW, | BW, |
|---|---|---|---|---|---|
| Treat | Phytase¹ | TM² | kg 14 d | kg 21 d | kg 42 d |
| Con 1 | − | − | 9.53b | 11.14b | 24.48b |
| Con 1 + TM | − | + | 10.00a | 12.72a | 26.52a |
| CON2 | + | − | 9.89ab | 12.79a | 26.31a |
| SEM | | | 0.112 | 0.236 | 0.733 |
| P-value | | | 0.0238 | <.0001 | 0.0157 |

| Phase 1 | Phytase | TM | ADG | ADFI | FCR |
|---|---|---|---|---|---|
| Con 1 | − | − | 180.5b | 252.1 | 1.399 |
| Con 1 + TM | − | + | 213.9a | 282.7 | 1.335 |
| CON2 | + | − | 206.2ab | 282.6 | 1.373 |
| SEM | | | 8.03 | 12.95 | 0.026 |
| P-value | | | 0.0238 | 0.173 | 0.227 |

| Phase 2 | Phytase | TM | ADG | ADFI | FCR |
|---|---|---|---|---|---|
| Con 1 | − | − | 711.9 | 1150.8 | 1.624 |
| Con 1 + TM | − | + | 786.7 | 1256.6 | 1.605 |
| CON2 | + | − | 780.9 | 1259.7 | 1.620 |
| SEM | | | 32.64 | 41.67 | 0.022 |
| P-value | | | 0.0375 | 0.1252 | 0.820 |

| Overall | Phytase | TM | ADG | ADFI | FCR |
|---|---|---|---|---|---|
| Con 1 | − | − | 416.1b | 659.4 | 1.589 |
| Con 1 + TM | − | + | 464.8a | 722.5 | 1.563 |
| CON2 | + | − | 459.6a | 724.0 | 1.581 |
| SEM | | | 17.46 | 23.03 | 0.020 |
| P-value | | | 0.0157 | 0.092 | 0.655 |

¹Supplementation with a novel consensus phytase (PhyG) at 1500, 1000 FTU/kg in phase 1 and 2, respectively
²Supplemented with trace minerals including 120 ppm Zn from ZnSO₄, 80 ppm Cu from CuSO₄, 96 ppm Fe from FeSO₄, 80 ppm Mn from MnSO₄

Blood trace mineral content: The effect of supplementation with trace minerals, phytase or both, on the concentrations of trace minerals in the blood, is presented in Table 25. Supplemental phytase without supplemental TM in CON2 numerically increased Mn and Zn vs. CON1 without TM, but the differences were not statistically significant.

TABLE 25
Effect of supplementation with trace minerals or phytase
or both, on blood trace minerals concentration at
the end of study (42 d on trial), 1-way ANOVA

| | Phy- | | Cu | Zn | Mn | Fe | Se |
|---|---|---|---|---|---|---|---|
| | tase¹ | TM² | (mg/L) | (mg/L) | (ug/L) | (mg/L) | (ug/L) |
| Con 1 | − | − | 1.57 | 2.53 | 15.8 | 179.5 | 100 |
| Con 1 + | − | + | 1.52 | 2.50 | 14.9 | 169.3 | 100 |
| TM | | | | | | | |
| CON2 | + | − | 1.69 | 3.09 | 19.6 | 163.7 | 107 |
| SEM | | | 0.135 | 0.251 | 1.81 | 12.69 | 6.14 |
| P-value | | | 0.667 | 0.188 | 0.167 | 0.677 | 0.643 |

¹Supplementation with a novel consensus phytase (PhyG) at 1500, 1000 FTU/kg in phase 1 and 2, respectively
²Supplemented with trace minerals including 120 ppm Zn from ZnSO₄, 80 ppm Cu from CuSO₄, 96 ppm Fe from FeSO₄, 80 ppm Mn from MnSO₄

Conclusion: PhyG phytase supplemented to a corn-soybean meal-based diet totally replaced commercial levels of supplemental Zn, Fe, Cu and Mn in piglets from 7 to 25 kg of BW. The phytase dosed at 1500 FTU/kg during phase 1 and 1000 FTU/kg during phase 2 maintained body weight at levels equivalent to those produced by a nutritionally adequate diet supplemented with TM without phytase. These results indicate that PhyG phytase could be used to replace commercially applied levels of the TM including Zn, Fe, Cu and Mn in the diets of piglets.

### Example 5: Efficacy of a Novel Consensus Bacterial 6-Phytase Variant Included in Corn-Soybean Mcal-Based Dicts Formulated to Reduce the Level of Campylobacter in Broilers

This Example shows the effect of broiler diets specifically formulated without exogenous Fe and Inorganic Phosphate (IP-free) and supplemented with a novel consensus bacterial 6-phytase variant (PhyG; SEQ ID NO:26) on the prevalence and load of Campylobacter in broilers. All birds in the Example were challenged with Campylobacter jejuni on D14 of age via oral gavage and Campylobacter enumerated in the ceca of birds at slaughter age, D42. When fed to broilers, the “Test diet” free in added Fe and IP-free and supplemented with PhyG maintained growth performance throughout; there were more Campylobacter negative birds; fewer high colonizers (super shedders) and mean Campylobacter loads were reduced by approximately 1 Log₁₀.

### Materials and Methods

This study was carried out at the Southern Poultry Research Group (SPRG), in the state of Georgia, United States. Experimental protocols and procedures were evaluated and approved by the Ethical Committee on Animal Experiments of the research institute and complied with local legislation on the use of animals in research.

Experimental design, birds and housing: The experiment was carried out as a randomized design with 2 dietary treatments, 10 replicate floor-pens and 25 birds/pen. A total of 500 Ross 308 male broilers were assigned on day-of-hatch to pens so that each pen contained birds of approximately equal average bird weight. Pens were housed in a broiler house containing clean wood shavings as bedding. Temperature was maintained as per breeders recommendation for age of bird. Birds were provided lighting twenty-four (24) hours per day for the first forty-eight (48) hours and then lighting will be decreased to 20 hours per day until study termination.

Dietary treatments: Treatment diets were formulated in three phases: 1-14, 15-28 and 29-42 d of age as starter, grower and finisher phases, respectively. Treatments comprised:
1) Challenged control Positive Control diet; formulated to meet the nutrient requirements for broilers using Dicalcium phosphate and a premix containing added Fe;
2) Challenged Test diet; formulated to meet the nutrient requirements for broilers but without dicalcium phosphate and using an oxide-based premix not containing added Fe in all phases.
   The ingredient and calculated nutrient composition of the diets is given in Table 26 and the analysed total Fc content given in Table 27. The total Fe content is the sum of the added Fc content via the premix and the Fe that is part of the basic raw materials. The measured total Fe level confirmed the lack of addition of Fe via the premix leading to a significant reduction in total Fe level in the test diets.

TABLE 26
Ingredient and calculated nutrient composition of the two diets used in Example 5

| | Starter (d 1-14) | | Grower (d 15-28) | | Finisher (d 29-42) | |
|---|---|---|---|---|---|---|
| Ingredients, % as is: | PC | Test diet | PC | Test diet | PC | Test diet |
| Maize | 33.84 | 38.90 | 39.89 | 44.97 | 44.19 | 48.09 |
| Wheat | 10.00 | 10.00 | 10.00 | 10.00 | 10.00 | 10.00 |
| Soybean Meal | 34.59 | 34.58 | 30.49 | 29.15 | 26.73 | 26.24 |
| Rapeseed Meal 38.8% CP | 5.00 | 5.00 | 5.00 | 5.00 | 5.00 | 5.00 |
| Sunflower Meal 38.2% CP | 2.26 | 2.18 | 1.83 | 2.01 | 2.56 | 2.56 |
| Maize Gluten Meal | 5.66 | 2.94 | 4.35 | 2.52 | 3.40 | 1.58 |
| Soy oil | 4.87 | 3.59 | 5.10 | 3.80 | 5.26 | 4.31 |
| Limestone | 0.27 | 1.46 | 0.52 | 1.24 | 0.64 | 1.01 |
| Dicalcium Phosphate | 1.91 | — | 1.26 | — | 0.77 | — |
| L-Lysine HCL | 0.34 | 0.29 | 0.29 | 0.27 | 0.27 | 0.24 |
| DL-Methionine | 0.31 | 0.29 | 0.28 | 0.26 | 0.26 | 0.25 |
| L-Threonine | 0.13 | 0.11 | 0.11 | 0.09 | 0.09 | 0.08 |
| TM Premix - With Fe | 0.17 | — | 0.12 | — | 0.12 | — |
| TM Premix - Without Fe | — | 0.17 | — | 0.12 | — | 0.12 |
| Broiler vitamin premix | 0.05 | 0.05 | 0.05 | 0.05 | 0.05 | 0.05 |
| Amprolium - coccidiostat | 0.01 | 0.01 | 0.05 | 0.05 | 0.05 | 0.05 |
| Sodium Bicarbonate | 0.44 | 0.25 | 0.42 | 0.24 | 0.41 | 0.23 |
| NaCl | 0.18 | 0.19 | 0.20 | 0.19 | 0.20 | 0.20 |
| PhyG FTU/kg | 1000 | 3000 | 1000 | 2000 | 1000 | 2000 |
| ME Poultry - kcal/kg | 2945 | 2896 | 3020 | 2977 | 3070 | 3036 |
| Crude Fat | 7.93 | 6.75 | 8.21 | 7.03 | 8.48 | 7.61 |
| Starch | 28.84 | 31.50 | 32.33 | 35.15 | 34.61 | 36.71 |
| Calcium | 0.80 | 0.71 | 0.70 | 0.61 | 0.60 | 0.52 |
| Av. Phosphorus - P | 0.35 | 0.14 | 0.27 | 0.13 | 0.21 | 0.12 |
| Total Phosphorus | 0.81 | 0.42 | 0.65 | 0.40 | 0.55 | 0.39 |
| Crude Protein | 25.07 | 23.71 | 22.75 | 21.44 | 20.98 | 19.91 |
| Dig Lys - P | 1.32 | 1.27 | 1.18 | 1.13 | 1.08 | 1.04 |
| Dig Met & Cys - P | 1.00 | 0.94 | 0.92 | 0.86 | 0.86 | 0.81 |
| Dig Thr - P | 0.88 | 0.83 | 0.79 | 0.74 | 0.72 | 0.68 |
| Dig Trp - P | 0.23 | 0.23 | 0.21 | 0.20 | 0.19 | 0.19 |
| Dig Arg - P | 1.40 | 1.37 | 1.27 | 1.22 | 1.17 | 1.14 |
| Dig Val - P | 1.00 | 0.95 | 0.91 | 0.86 | 0.84 | 0.80 |
| Phytate Phosphorus | 0.31 | 0.31 | 0.30 | 0.30 | 0.29 | 0.29 |

TABLE 27
Analyzed total Fe mineral content of
the experimental diets in Example 5.

| | Starter (d 1-14) | | Grower (d 15-28) | | Finisher (d 29-42) | |
|---|---|---|---|---|---|---|
| | PC | Test diet | PC | Test diet | PC | Test diet |
| Iron, ppm | 208 | 134 | 163 | 106 | 146 | 80 |

Campylobacter challenge: On day of arrival, 5 additional birds from the batch were randomly euthanized and ceca, liver and spleen aseptically extracted and analyzed via culture dependent methods for Campylobacter to confirm Campylobacter negative. On day 7, the barn was confirmed Campylobacter negative by boot swab analysis. On d 14 all birds in the experiment were orally gavaged with 0.1 ml of a ~1.0×10⁶ CFU/ml (~1.0×10⁵ CFU/chick) of Campylobacter jejuni J.B. strain. At day 42, birds were processed as described below to determine Campylobacter colonization characteristics.

Sampling, measurements and statistical analysis: Performance metrics including BW, FI, FCR and liveability were measured at the end of each dietary phase on days 14, 28 and 42 d of age. Performance results were analyzed using One-way ANOVA (JMP 16.0). P<0.05 was considered statistically significant. 0.05≤P<0.1 was considered a tendency. On d 42, 10 birds from each pen (100 birds per treatment) were euthanized via cervical dislocation and the ceca aseptically removed and placed in sterile sampling bags for Campylobacter enumeration via culture dependent methods. In brief, 1 ml aliquot of stomached cecal samples in MRD broth was transferred into a 96 well plate. 10-fold serial dilutions proceeded and dilutions were then spread onto Campy-Cefex Agar plates and incubation at 42 C, microaerophilic conditions for 48 hours prior to enumeration. Statistical analysis of Ceca loads was evaluated using the Kruskal Wallis non-parametric analysis of variance [ANOVA]. P<0.05 was considered statistically significant.

### Results

Growth performance: The results for body weight (BW), BW gain (BWG), feed intake (FI) and feed conversion ratio (FCR) and liveability by phase and overall are presented in Table 28. Overall, the test diet (formulated without the addition of Fe, IP-free and using an oxide-based premix and PhyG) resulted in numerically higher BW at D 42 (2655.3 g vs 2524.9 g in PC), though this was not statistically different to the PC (P=0.11). The liveability was numerically improved from 94.60% to 96.80% at D 42.

TABLE 28
Effect of phytase supplementation on growth performance
by dietary phase and overall. One way ANOVA¹.

| | | | | | | |
|---|---|---|---|---|---|---|
| BW_start, | BW_end | FI | BWG | | | Liveability |
| (g/bird) | (g/bird) | (g/bird) | (g/bird) | FCR | FCRc² | (%, M/C) |

d 1-14

| | | | | | | |
|---|---|---|---|---|---|---|
| Positive control | 42.7 | 353.0 | 399.2 | 310.5 | 1.293 | 98.8 |
| Test diet | 42.4 | 375.1 | 444.6 | 332.7 | 1.348 | 98.4 |
| Pooled SEM | 0.225 | 7.140 | 5.368 | 7.121 | 0.022 | 0.632 |
| P-values | 0.328 | 0.042 | <.0001 | 0.041 | 0.087 | 0.660 |

d 15-28

| | | | | | | |
|---|---|---|---|---|---|---|
| Positive control | 353.0 | 1312.9 | 1331.4 | 959.8 | 1.396 | 98.4 |
| Test diet | 375.1 | 1400.5 | 1488.4 | 1025.4 | 1.453 | 99.6 |
| Pooled SEM | 7.140 | 20.902 | 20.407 | 14.772 | 0.010 | 0.547 |
| P-values | 0.043 | 0.008 | <.0001 | 0.006 | 0.001 | 0.132 |

d 29-42

| | | | | | | |
|---|---|---|---|---|---|---|
| Positive control | 1312.9 | 2524.9 | 1320.3 | 1211.9 | 1.745 | 97.5 |
| Test diet | 1400.6 | 2655.3 | 1479.1 | 1254.9 | 1.778 | 98.4 |
| Pooled SEM | 20.885 | 54.999 | 49.266 | 49.651 | 0.043 | 0.899 |
| P-values | 0.008 | 0.111 | 0.035 | 0.548 | 0.600 | 0.503 |

d 1-28

| | | | | | | | |
|---|---|---|---|---|---|---|---|
| Positive control | 42.7 | 1312.9 | 1730.6 | 1270.2 | 1.370 | 1.370 | 97.2 |
| Test diet | 42.4 | 1400.5 | 1933.1 | 1358.3 | 1.428 | 1.401 | 98.0 |
| Pooled SEM | 0.225 | 20.902 | 23.906 | 20.848 | 0.010 | 0.014 | 0.766 |
| P-values | 0.328 | 0.008 | <.0001 | 0.008 | 0.001 | 0.133 | 0.470 |

d 1-42

| | | | | | | | |
|---|---|---|---|---|---|---|---|
| Positive control | 42.7 | 2524.9 | 3050.8 | 2482.1 | 1.546 | 1.546 | 94.8 |
| Test diet | 42.4 | 2655.3 | 3412.1 | 2612.9 | 1.593 | 1.554 | 96.4 |
| Pooled SEM | 0.225 | 54.999 | 62.259 | 55.076 | 0.015 | 0.024 | 0.989 |
| P-values | 0.328 | 0.111 | 0.001 | 0.110 | 0.043 | 0.813 | 0.268 |

¹P < 0.05 was considered statistically significant. 0.05 ≤ P < 0.1 was considered a tendency
²FCRc = Body weight corrected FCR

Campylobacter was observed in birds fed the Test dict. Eighty-four out of 100 (84%) vs 88/100 (88%) ceca were positive for Campylobacter colonization in Test diet vs PC respectively. Both the Positive control and Test diet showed the degree of Campylobacter load within each ceca was varied, however the Test diet absent in IP, Fe and formulated with oxide-based premix, showed approximately a 1 Log₁₀ CFU reduction in the highest level achieved, reducing overall the high seeders of Campylobacter into the environment (2.45×10¹⁰ vs 2.97×10⁹ CFU/g in PC and Test diet respectively), though this was not statistically different (P>0.0.5) likely to the high variation in CFUs recorded. Additionally, mean Campylobacter loads were substantially reduced by almost 1 Log₁₀ in Test diets vs PC, however not statistically significant likely owing to the variation in the overall colonization levels (3.78×10⁸ CFU/g vs 4.90×10⁷ CFU/g in PC and Test diet respectively).

The impact of Test diet formulation on reducing Campylobacter colonisation is visualized in Table 29. Test diet resulted in fewer (2 vs 6% of total birds sampled) high colonisers above the 1×10⁸ CFU/g threshold. Furthermore, Test diet resulted in a reduction in the second defined threshold for Campylobacter colonisation 10⁶ CFU per gram ceca. Thirty percent of PC birds harboured levels of over 10⁶ CFU per gram, vs only 20% in the test diet fed treatment.

TABLE 29
Categorized Campylobacter loads in the ceca of broilers D42 (% positive birds)

| | % of positive birds with Campylobacter ceca loads in each respective range (%) | | | | | |
|---|---|---|---|---|---|---|
| | | | 1 × 10⁵- | 1 × 10⁶- | 1 × 10⁷- | |
| Treatment | Negative | <1 × 10⁵ | 1 × 10⁶ | 1 × 10⁷ | 1 × 10⁸ | >1 × 10⁸ |
| Positive Control | 12 | 20 | 38 | 17 | 7 | 6 |
| Test Diet | 16 | 25 | 40 | 11 | 7 | 2 |

Conclusion: Overall, diets formulated without the addition of Fe, inorganic phosphate and utilising oxide-based premix and PhyG resulted in; a reduction in the mean Campylobacter load at D 42 to a level below the suggested critical threshold for processing contamination (1×10⁸ CFU/g of ceca); a reduction in the subsequent stretch goal (1×10⁶ CFU/g ceca) to further reduce Campylobacter spread at the processing house; a reduction in super high colonizers and potential super shedders to drive spread of infection whilst maintaining final bodyweight, a critical production metric.

Effective Campylobacter control is multipronged and reducing levels of Campylobacter at the farm will result in less transmission and contamination at the slaughter/processing house, fundamentally reducing the likelihood edible poultry tissues are contaminated with Campylobacter and pose a foodborne illness risk following human handling and consumption.

### REFERENCES

Adeola et al. 1995. J. Anim. Sci. 73:3384-3391.
Bao et al. 2007. J. Appl. Poult. Res. 16:448-455.
Bao et al. 2009. Brit. Poult. Sci. 1:95-102.
Bao et al. 2010i 1:109-117.
Christensen et al. 2020. Curr. Biochem. Eng. 6:156-171.
Dersjant-Li et al. 2022a. Brit. Poult. Sci. 63:395-405.
Dersjant-Li et al. 2022b. Poult. Sci. 101:101666.
Dersjant-Li et al., 2020. Anim. Feed Sci. Technol. 264:114481.
Humer et al. 2015. J. Anim. Physiol. Anim. Nutr. (Berl). 99:605-625.
Jondreville et al. 2007. Animal 1:804-811.
Lei et al. 1993. J. Nutr. 123:1117-1123.
Li et al. 2016. Poult. Sci. 95:581-589.
Scbastian et al. 1996. Poult. Sci. 75:729-736.
Selle and Ravindran 2007. Anim. Feed Sci. Technol. 135:1-41.
Selle et al. 2009. Livest. Sci. 124:126-141.
Sharpley et al. 1994. J. Environ. Qual. 23:437-451.
Singh et al. 2015. Biol. Trace Elem. Res. 164:253-260.
Suttle, N. F., 2010. Mineral Nutrition of Livestock. 4th edition, CABI, Cambridge.
Tamim et al. 2004. Poult. Sci. 83:1358-1367.
Yi et al. 1996. Poult. Sci. 75:540-546.

We claim:

1. An animal diet comprising (i) an engineered phytase polypeptide or a fragment thereof comprising phytase activity; and (ii) lacking one or more exogenously added trace minerals.

2. The diet of claim 1, wherein the one or more trace mineral is one or more trace mineral selected from the group consisting of zinc (Zn), iron (Fe), copper (Cu), manganese (Mn) and selenium (Se).

3. The diet of claim 1 or claim 2, wherein the phytase polypeptide or a fragment thereof comprising phytase activity comprises at least 82% sequence identity with the amino acid sequence set forth in SEQ ID NO:1.

4. The diet of any one of claims 1-3, wherein said phytase polypeptide comprises an amino acid sequence selected from the group consisting of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO: 12, SEQ ID NO: 13, SEQ ID NO: 14, SEQ ID NO:16, SEQ ID NO: 17, SEQ ID NO:18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO: 28, SEQ ID NO:29, SEQ ID NO:30, SEQ ID NO:31, SEQ ID NO:32, SEQ ID: NO: 33, SEQ ID NO: 34, SEQ ID NO:35, SEQ ID NO:36, and SEQ ID NO:37.

5. The diet any one of claims 1-4 further comprising one or more of (a) a direct fed microbial comprising at least one bacterial strain, (b) at least one other enzyme, and/or (c) an essential oil.

6. The diet of any one of claims 1-5, wherein the engineered phytase polypeptide or fragment thereof is present in an amount of at least about 0.1 g/ton feed.

7. The diet of any one of claims 1-6 comprising calcium from about 0.62 to 0.72% in a starter diet, about 0.54 to 0.64% in a grower diet, and/or about 0.42 to 0.55% in a finisher diet.

8. The diet of claim 7, wherein the finisher diet comprises about 0.46 to 0.55% or from about 0.42 to 0.50% calcium.

9. The diet of any one of claims 1-8, comprising amino acids from about 1.18 to 1.22% in a starter diet, about 1.06 to 1.10% in a grower diet, and/or about 0.88 to 1% in finisher diet.

10. The diet of claim 9, wherein the finisher diet comprises about 0.96 to 1.0% or from about 0.88 to 0.92% amino acids.

11. The diet of claim 9 or claim 10, wherein the amino acids comprise digestible lysine.

12. The diet of any one of claims 1-11, comprising metabolizable energy from about 2824 to 2950 kcal/kg in a starter diet, about 2924 to 3050 kcal/kg in a grower diet, and/or about 2970 to 3120 kcal/kg in a finisher diet.

13. The diet of claim 12, wherein the finisher diet comprises about 2970 to 3100 kcal/kg or from about 2994 to 3120 kcal/kg metabolizable energy.

14. The diet of any one of claims 1-13, comprising sodium from about 0.13 to 0.17%.

15. The dict of any one of claims 1-14, wherein the phytase is present at a dose of between about 500 FTU/kg to about 2000 FTU/kg.

16. The diet of any one of claims 1-15, wherein the diet contains a phytate source comprising one or more of corn, wheat, soybean meal, rapeseed, rice and/or wheat bran.

17. The diet of any one of claims 1-16, wherein the diet further comprises oat hulls.

18. The diet of any one of claims 1-17, wherein the diet lacks meat and/or bone meal.

19. The diet of any one of claims 1-18, further comprising one or more additional feed enzymes selected from the group consisting of a xylanase, a protease, an amylase, and a glucoamylase.

20. The diet of any one of claims 1-19, wherein the diet is a starter diet.

21. The diet of any one of claims 1-20, wherein the diet is a grower diet.

22. The diet of any one of claims 1-21, wherein the diet is a finisher diet.

23. The diet of any one of claims 1-22, wherein the animal is poultry.

24. The diet of any one of claims 1-22, wherein the animal is swine.

25. The diet of any one of claims 1-22, wherein the animal is a ruminant.

26. The diet of claim 25, wherein the ruminant is a calf.

27. The diet of any one of claims 1-26, wherein the diet further contains no or substantially no exogenously-added inorganic phosphate.

28. A method for improving animal performance on one or more metrics comprising administering an effective amount of the diet of any one of claims 1-27 to an animal.

29. The method of claim 28, wherein the one or more metrics is selected from the group consisting of increased feed efficiency, increased weight gain, reduced feed conversion ratio, improved digestibility of nutrients or energy in a feed, improved nitrogen retention, improved ability to avoid the negative effects of necrotic enteritis, and improved immune response.

30. The method of claim 28 or claim 29, wherein the animal is poultry, swine, or a ruminant animal.

31. The method of claim 30, wherein the poultry is selected from the group consisting of turkeys, ducks, chickens, geese, pheasants, quail, and emus.

32. The method of claim 31, wherein the chicken is selected from the group consisting of layers and broilers.

33. A method for reducing pathogenic bacteria populations in animals comprising administering an effective amount of the diet of any one of claims 1-27 to an animal.

34. The method of claim 33, wherein a) the one or more trace mineral is iron (Fe); and/or b) the dict further contains no or substantially no exogenously-added inorganic phosphate.

35. The method of claim 33 or claim 34, wherein the pathogenic bacterial population is one or more bacteria selected from the group consisting of Actinobacillus, Bordetalla, Campylobacter, Clostridium, Corynebacterium, Escherichia coli, Globicatella, Listeria, Mycobacterium, Salmonella, Staphylococcus, and Streptococcus.

36. The method of any one of claims 34-35, wherein the animal is poultry, swine, or a ruminant animal.

37. The method of claim 36, wherein the poultry is selected from the group consisting of turkeys, ducks, chickens, geese, pheasants, quail, and emus.

38. The method of claim 36, wherein the chicken is selected from the group consisting of layers and broilers.

39. The method of claim 36, wherein the ruminant animal is a calf.


SEQUENCE LISTING (37 sequences)

SEQ ID NO: 1 - protein, 413 aa, synthetic construct
  REGION 1..413: note = PHY-13594
SEAAPAGYHLDKAVILSRHGVRAPTKMTQTMRDVTPYTWPEWPVPLGYITPRGEHLVSLMGGFYRQYFQQQGLLSRDSCPTANDVYVWTDVNQRTRKTGEAFLAGLAPECDLTIHHQNDIKQVDPLFHPVKAGICSMDKTQVQQAVEKQAGMPIDKLNQHYRPELALMSNVLNFPKSPYCQRHSGEQPCDFANAFPSKLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 2 - protein, 413 aa, synthetic construct
  REGION 1..413: note = PHY-10931
SETAPAGYHLDKAVILSRHGVRAPTKMTQLMRDVTPNQWPEWPVPLGYITPRGEHLVSLMGGFYRQYFQQQGLLSRDRCPTANDVYVWTDVNQRTRKTGEAFLAGLAPQCPLTIHHQSNIKVVDPLFHPLKTGICSMDKTQVQQAVEKQAGMPIDKLNQYYAPELSLMSNVLNFPKSPYCRQHSVEKPCDFAQMFPSKLNISDDGNEVQLEGAVGLSSTLAEIFLLEHAQGMPVVAWGNIHSEQEWNDLLKLHNAQFDLMERTPYIAKHNGTPLLQTIVNALNSNTTARELPDISPDNKILFLAGHDTNIANIGGMLGMTWTLPGQPDNTPPGGALLFELWSDKAGTQYVSVHMVYQTLAQLRNQTPLTLKEPAGSVPLKIPGCDDQTAEGYCPLDTFTRLVNQVVEPACQLP

SEQ ID NO: 3 - protein, 413 aa, synthetic construct
  REGION 1..413: note = PHY-10957
SEAAPSGYHLQKAVILSRHGVRAPTKMTQLMRDVTPYTWPEWPVPLGYITPRGEHLVSLMGGFYRQWFQQQGILSKERCPTANDVYVWTDVNQRTRKTGEAFLAGLAPKCPLTIHHQSNIKVVDPLFHPLKTGICSMDKTQVQQAVEKQAGMPIDKLNQHYRPELALMSNVLNFPKSPYCRQHSVEKPCDFANAFPSKLNITDDGNEVQLEGAVGLSSTLAEIFLLEYAQGMPVVAWGNIHSEQEWNALLKLHNAYFDLMERTPYIAKHNGTPLLQTIVNALNSNTTARELPDISPDVKILFLAGHDTNIANIAGMLGMDWTLPGQPDNTPPGGALLFELWRDKEGTQYVSVKMVYQTLAQLRNQTPLTLKEPAGSVPLKIPGCDDQTAEGYCPLVTFTRLVNQVVEPACQIP

SEQ ID NO: 4 - protein, 413 aa, synthetic construct
  REGION 1..413: note = PHY-11569
SETAPSGYHLDKAVILSRHGVRAPTKMTQLMRDVTPYTWPEWPVPLGYITPRGEHLVSLMGGFYRQYFQQQGLLSKDNCPTANDVYVWTDVDQRTRKTGEAFLAGLAPECDLTIHHQNNIKVVDPMFHPLKTGICSMDKTQVQQAVEKQAGMPIDKLNQHYAPELALMSAVLNFPASPYCQQHSVDKPCDFANAFPSKLNITDDGNEVQLEGAVGLSSTLAEIFLLEYAQGMPVVAWGNIHSEQEWNALLKLHNAHFDLMSRTPYIAKHNGTPLLQTIVNALNSNTTARELPDISPDNKILFLAGHDTNIANIGGMLGMDWTLPGQPDNTPPGGALLFELWRDKAGTQYVSVHMVYQTLAQLRNQTPLTLKEPAGTVPLKIPGCDDQTAEGYCPLVTFTRLVNQSVEPACQLP

SEQ ID NO: 5 - protein, 413 aa, synthetic construct
  REGION 1..413: note = PHY-11658
SEAAPSGYHLDKAVLLSRHGVRAPTKMTQLMRDVTPYTWPEWPVPLGYITPRGEHLVSLMGGFYRQWFQQQGLLSKDRCPTANDVYVWADVGQRTRKTGEAFLAGLAPECDLTIHHQNDIKQVDPMFHPLKAGICSMDKTQVQQAVEKQAGMPIDKLNQHYRPELALMSNVLNFPKSPYCRQHSVEKPCDFAQMFPSKLNITDDGNEVQLEGAVGLSSTLAEIFLLEYAQGMPVVAWGNIHSEQEWNDLLKLHNAQFDLMQRTPYIAKHNGTPLLQTIVNALNSNTTARELPDISPDVKILFLAGHDTNIANIGGMLGMDWTLPGQPDNTPPGGALLFERWSDKDGTQYVSVHMVYQTLAQLRNQTPLTLKEPAGTVPLKIPGCDDQTAEGYCPLVTFTRLVNQSVEPACQLP

SEQ ID NO: 6 - protein, 413 aa, synthetic construct
  REGION 1..413: note = PHY-11673
SEAAPSGYHLDKAVILSRHGVRAPTKMTELMRDVTPYTWPEWPVPLGYITPRGEHLVSLMGGFYRQYFQQQGLLSKDRCPTANDVYVWTDVNQRTRKTGEAFLAGLAPECPLTIHHQSNIKVVDPLFHPVKTGLCSMDKTQVQQAVEKQAGMPIDKLNDKYAPELALMSNVLNFPKSPYCRQHSVEKPCDFAQMFPSKLSIGDDGNEVQLEGAVGLSSTLAEIFLLEYAQGMPVVAWGNIHSEQEWNDLLKLHNAHFDLMERTPYIAKHNGTPLLQTIVNALNSNTTESKLPDISPSVKILFLAGHDTNIANIGGMLGMDWTLPGQPDNTPPGGALLFELWRDKDGTQYVSVHMVYQTLAQLRNQTPLTLKEPAGTVPLKIPGCDDQTAEGYCPLVTFTVLVNQSVEPACQLP

SEQ ID NO: 7 - protein, 413 aa, synthetic construct
  REGION 1..413: note = PHY-11680
SETAPSGYHLDKAVILSRHGVRAPTKMTQLMRDVTPYTWPEWPVPLGYITPRGEHLVSLMGGFYRQYFQQQGLLSKDNCPTANDVYVWTDVDQRTRKTGEAFLAGLAPECDLTIHHQNNIKVVDPLFHPLKTGICSMDKTQVQQAVEKQAGMPIDKLNDHYRPELSLMSNVLNFPKSPYCQRHSGEKTCDFANAFPSKLIITDDGNKVALEGAVGLSSTLAEIFLLEYAQGMPKVAWGNIHSEQEWNDLLKLHNAQFDLMERTPYIAKHNGTPLLQTIVNALNSNTTARELPDISPDVKILFLAGHDTNIANIGGMLGMDWTLPGQPDNTPPGGALLFELWRDKEGTQYVSVKMVYQTLAQLRNQTPLTLKEPAGSVPLKIPGCDDQTAEGYCPLDTFTRLVNQVVEPACQIP

SEQ ID NO: 8 - protein, 413 aa, synthetic construct
  REGION 1..413: note = PHY-11895
SEAAPSGYHLDKAVLLSRHGVRAPTKMTQLMRDVTPYTWPEWPVPLGYITPRGEHLVSLMGGFYRQWFQQQGLLSRDRCPTANDVYVWTDVNQRTRKTGEAFLAGLAPECDLTIHHQKNIKVVDPLFHPLKAGICSMDKTQVQQAVEKQAGMPIDKLNQHYAPELSLMSNVLNFPKSPYCQRHSGEKTCDFANAFPSKLNISDDGNEVQLEGAVGLSSTLAEIFLLEYAQGMPVVAWGNIHSEQEWNALLKLHNAHFDLMERTPYIAKHNGTPLLQTIVNALNSNTTARELPDISPSVKILFLAGHDTNIANIGGMLGMTWTLPGQPDNTPPGGALLFELWSDKAGTQYVSVHMVYQTLTQLRNQTPLTLKEPAGSVPLKIPGCDDQTAEGYCPLDTFTRLVNQVVEPACQLP

SEQ ID NO: 9 - protein, 413 aa, synthetic construct
  REGION 1..413: note = PHY-11932
SETAPAGYHLDKAVILSRHGVRAPTKMTQTMRDVTPYTWPEWPVPLGYITPRGEHLVSLMGGFYRQYFQQQGLLSAERCPTANDVYVWTDVNQRTRKTGEAFLAGLAPQCPLTIHHQNDIKQVDPMFHPLKAGICSMDKTQVQQAVEKQAGMPIDKLNDHYRPELSLMSNVLNFPKSPYCARHSGDKPCDFAQMMPSKLNITDDGNEVQLEGAVGLSSTLAEIFLLEYAQGMPVVAWGNIHSEQEWNALLKLHNAQFDLMERTPYIAKHNGTPLLQTIVNALNSNTTARELPDISPDVKILFLAGHDTNIANIAGMLGMTWTLPGQPDNTPPGGALLFELWSDKAGTQYVSVHMVYQTLAQLRNQTPLTLKEPAGSVPLKIPGCDDQTAEGYCPLDTFTRLVNQVVEPACQLP

SEQ ID NO: 10 - protein, 413 aa, synthetic construct
  REGION 1..413: note = PHY-12058
SEAAPAGYHLDKVVILSRHGVRAPTKMTQTMRDVTPYTWPEWPVPLGYITPRGEHLVSLMGGFYRQKFQQQGLLSKDRCPTANDVYVWTDVDQRTRKTGEAFLAGLAPKCPLTIHHQSNIKVVDPLFHPLKTGICSMDKTQVQQAVEKQAGMPIDKLNQHYRPELSLMSNVLNFPKSPYCAQHSGEKPCDFAQMFPSKLNISDDGNEVQLEGAVGLSSTLAEIFLLEYAQGMPVVAWGNIHSEQEWNDLLKLHNAYFDLMERTPYIAKHNGTPLLQTIVNALNSNTTARELPDISPSVKILFLAGHDTNIANIGGMLGMDWTLPGQPDNTPPGGALLFELWRDKDGTQYVSVKMVYQTLAQLRNQTPLTLKEPAGSVPLKIPGCDDQTAEGYCPLDTFTRLVNQSVEPACQIP

SEQ ID NO: 11 - protein, 413 aa, synthetic construct
  REGION 1..413: note = PHY-12663
SEAAPSGYHLDKVVILSRHGVRAPTKMTQLMRDVTPYTWPEWPVPLGYITPRGEHLVSLMGGFYRQWFQQQGLLSKDRCPTANDVYVWTDVNQRTRKTGEAFLAGLAPQCPLTIHHQKNIKVVDPLFHPLKTGICSMDKTQVQQAVEKQAGMPIDKLNQHYRPELALMSNVLNFPKSPYCQRHSGDEPCDFANAFPSKLNITDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDNKILFLAGHDTNIANIGGMLGMDWTLPGQPDNTPPGGALLFERWRDKEGTQYVSVHMVYQTLAQLRNQTPLTLKEPAGSVPLKIPGCDDQTAEGYCPLDTFTRLVNQVVEPACQIP

SEQ ID NO: 12 - protein, 413 aa, synthetic construct
  REGION 1..413: note = PHY-12784
SEAAPSGYHLDKAVLLSRHGVRAPTKMTQLMRDVTPNQWPEWPVPLGYITPRGEHLVSLMGGFYRQYFQQQGILSKDSCPTANDVYVWTDVNQRTRKTGEAFLAGLAPQCPLTIHHQSDIKQVDPMFHPLKAGICSMDKTQVQQAVEKQAGMPIDKLNQYYAPELALMSNVLNFPKSPYCQRHSGEQTCDFAQMFPSKLNISDDGNEVQLEGAVGLSSTLAETFLLEYAQGMPVVAWGNIHSEQEWNALLKLHNAQFDLMQRTPYIAKHNGTPLLQTIVNALNSNTTARELPDISPSVKILFLAGHDTNIANIGGMLGMDWTLPGQPDNTPPGGALLFELWRDKEGTQYVSVHMVYQTLTQLRNQTPLTLKEPAGTVPLKIPGCDDQTAEGYCPLVTFTRLVNQVVEPACQIP

SEQ ID NO: 13 - protein, 413 aa, synthetic construct
  REGION 1..413: note = PHY-13177
SEAAPSGYHLDKVVILSRHGVRAPTKMTQLMRDVTPNQWPEWPVPLGYITPRGEHLVSLMGGFYRQYFQQQGLLSKDRCPTANDVYVWADVNQRTRKTGEAFLAGLAPRCDLTIHHQKDIKQVDPMFHPLKAGICSMDKTQVQQAVEKQAGMPIDKLNQHYRPELALMSNVLNFPKSPYCAQHSGEKPCDFAQMFPSKLNISDDGNEVQLEGAVGLSSTLAEIFLLEYAQGMPVVAWGNIHSEQEWNDLLKLHNAQFDLMQRTPYIAKHNGTPLLQTIVNALNSNTTARELPDISPDNKILFLAGHDTNIANIGGMLGMDWTLPGQPDNTPPGGALLFERWRDKAGTQYVSVHMVYQTLAQLRNQTPLTLKEPAGTVPLKIPGCDDQTAEGYCPLDTFTRLVNQVVEPACQLP

SEQ ID NO: 14 - protein, 413 aa, synthetic construct
  REGION 1..413: note = PHY-13371
SEAAPAGYHLQKAVILSRHGVRAPTKMTQTMRDVTPYTWPEWPVPLGYITPRGEHLVKLMGGFYRQYFQQQGLLSRDRCPTANDVYVWTDVNQRTRKTGEAFLAGLAPECDLTIHHQSDIKQVDPLFHPVKAGICSMDKTQVQQAVEKQAGMPIDKLNQHYRPELALMSNVLNFPKSPYCQQHSVEQPCDFANAFPSYLNISDDGNEVQLEGAVGLGSTLAEIFLLEYAQGMPVVAWGNIHSEQEWNALLKLHNAHFNLMERTPYIAKHNGTPLLQTIVNALNSNTTARELPDISPDVKILFLAGHDTNIANIGGMLGMTWTLPGQPDNTPPGGALLFELWSDKEGTQYVAVKMVYQTLAQLRNQTPLTLKEPAGTVPLKIPGCDDQTAEGYCPLDTFTRLVNQVIEPACQLP

SEQ ID NO: 15 - protein, 413 aa, synthetic construct
  REGION 1..413: note = PHY-13460
SEAAPSGYHLQKAVILSRHGVRAPTKMTQLMRDVTPYTWPEWPVPLGYITPRGEHLVKLMGGFYRQYFQQQGLLSRDNCPTANDVYVWTDVAQRTRKTGEAFLAGLAPECDLTVHHQNDIKQVDPLFHPVKAGICSMNKSQVQQAVEKQAGMPIDKLNQHYRPELALMSNVLNFPKSPYCQRHSGEKPCDFAQMMPSKLNISDDGNEVQLEGAVGLSSTLAEIFLLEYAQGMPVVAWGNIHSEQEWNALLKLHNAQFDLMERTPYIAKHNGTPLMQTIVNALNSNTTARELPDISPDVKILFLAGHDTNIANIAGMLGMTWTLPGQPDNTPPGGALLFELWSDKEGTQYVSVKMVYQTLAQLRNQTPLTLKEPAGTVPLKIPGCDDQTAEGYCPLDTFTRLVNQVVEPACQLP

SEQ ID NO: 16 - protein, 413 aa, synthetic construct
  REGION 1..413: note = PHY-13513
SEAAPSGYHLQKAVILSRHGVRAPTKMTQLMRDVTPYTWPEWPVPLGYITPRGEHLVKLMGGFYRQYFQQQGLLSRDSCPTANDVYVWTDVNQRTRKTGEAFLAGLAPGCDLTIHHQSDIKQADPLFHPVKAGICSMDKTQVQQAVEKQAGGPIETLNQRYQAELALMSSVLDFPKSPYCQRHSVDKPCDFANAFPSYLNISDDGNEVQLEGAVGLSSTLAEIFLLEYAQGMPVVAWGNIHSEQEWNALLKLHNAHFDLMERTPYIAKHNGTPLLQTIVNALNSNTTARELPDISPDVKILFLAGHDTNIANIGGMLGMTWTLPGQPDNTPPGGALLFELWRDKDGTQYVSVHMVYQTLAQLRNMTPLTLKEPAGSVPLKIPGCDDQTAEGYCPLDTFTRLVNQVVEPACQLP

SEQ ID NO: 17 - protein, 413 aa, synthetic construct
  REGION 1..413: note = PHY-13637
SEAAPAGYHLQKAVILSRHGVRAPTKMTQLMRDVTPYTWPEWPVPLGYITPRGEHLVSLMGGFYRQYFQQQGLLSKDNCPTANDVYVWTDVNQRTRKTGEAFLAGLAPECDLTIHHQNDIKQVDPLFHPLKAGICSMDKTQVQQAVEKQAGMPIDKLNQHYRPELALMSSVLDFPKSPYCQHHSGDQPCDFAQMFPSYLNISDDGNEVQLEGAVGLSSTLAEIFLLEYAQGMPVVAWGNIHSEQEWNDLLKLHNAYFDLMERTPYIAKHNGTPLLQTIVNALNSNTTARELPDVSPDVKILFLAGHDTNIANIGGMLGMDWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 18 - protein, 413 aa, synthetic construct
  REGION 1..413: note = PHY-13705
SEAAPAGYHLDKAVILSRHGVRAPTKMTQLMRDVTPYTWPEWPVPLGYITPRGEHLVKLMGGFYRQYFQQQGLLSRDSCPTANDVYVWTDVNQRTRKTGEAFLAGLAPECDLTIHHQSDIKQADPLFHPLKAGICSMDKTQVQQAVEKQAGMPIDKLNQHYRAELALMSNVLNFPKSPYCRQHSVEQPCDFANAFPSRLNISDDGNEVQLEGAVGLSSTLAEIFLLEYAQGMPVVAWGNIHSEQEWNDLLKLHNAYFDLMERTPYIAKHQGTPLLQTIVNALNSNTTARELPDISPDVKILFLAGHDTNIANIAGMLGMDWTLPGQPDNTPPGGALLFELWSDKEGTQYVSVKMVYQTLAQLRNQTPLTLKEPAGSVPLKIPGCDDQTAEGYCPLDTFTRLVNRVVEPACQLP

SEQ ID NO: 19 - protein, 413 aa, synthetic construct
  REGION 1..413: note = PHY-13713
SEAAPAGYHLQKAVILSRHGVRAPTKMTQLMRDVTPYTWPEWPVPLGYITPRGEHLVSLMGGFYRQYFQQQGLLSRDRCPTPDAVYVWTDVNQRTRKTGEAFLAGLAPECDLTIHHQNDIHQVDPLFHPVKAGICSMDKTQVQQAVEKQAGMPIDKLNQHYRPELALMSNVLNFPKSPYCQRHSVDKPCDFAQMMPSYLNISDDGNEVQLEGAVGLSSTLAEIFLLEYAQGMPVVAWGNIHSEQEWNDLLKLHNAHFDLMERTPYIAKHQGTPLLQTIVNALNSNTTESKLPDISPDVKILFLAGHDTNIANIAGMLGMTWTLPGQPDNTPPGGALLFELWRDKAGTQYVSVKMVYQTLAQLRNQTPLTLKEPAGSVPLKIPGCDDQTAEGYCPLDTFTRLVNQVVEPACQLP

SEQ ID NO: 20 - protein, 412 aa, synthetic construct
  REGION 1..412: note = PHY-13747
SEAAPSGYHLQKAVILSRHGVRAPTKMTQLMRDVTPYTWPEWPVPLGYITPRGEHLVSLMGGFYRQYFQQQGLLSKDNCPTANDVYVWTDVNQRTRKTGEAFLAGLAPQCDLAIHHQNDIKQVDPLFHPLKAGICSMDKTQVQQAVEKQAGMPIDKLNQHYRPELALMSNVLNFPKSPYCRRHSGEKPCDFAQMMPSKLNISDDGNEVQLEGAVGLSSTLAEIFLLEYAQGMPVVAWGNIHSEEEWNDLLKLHNAYFDLMQRTPYIAKHNGTPLMQTISNALNPNATESKLPDISPDNKILFIAGHDTNIANIGGMLGMTWTLPGQPDNTPPGGALLFELWSDKEGTQYVSVKMVYQTLAQLRNQTPLTLKEPAGTVPLKIPGCDDQTAEGYCPLDTFTRLVNQVVEPACQI

SEQ ID NO: 21 - protein, 412 aa, synthetic construct
  REGION 1..412: note = PHY-13779
SEAAPAGYHLQKAVILSRHGVRAPTKMTQTMRDVTPHQWPEWPVPLGYITPRGEHLVKLMGGFYRQYFQQQGLLSKDRCPTANDVYVWTDVNQRTRKTGEAFLAGLAPECDLTIHHQSDIKQVDPLFHPLKAGICSMDKTQVQQAVEKQAGMPIDELNQHYRPELALMSNVLNFPKSPYCQRHSGEQPCDFANAFPSYLNISDDGNEVQLEGAVGLSSTLAEIFLLEYAQGMPVVAWGNIHSEQEWNDLLKLHNAYFDLMQRTPYIAKHNGTPLLQTIVNALNSNTTARELPDISPDVKILFLAGHDTNIANIGGMLGMTWTLPGQPDNTPPGGALLFELWSDKAGTQYVSVHMVYQTLAQLRNMTPLTLKEPAGTVPLKIPGCDDQTAEGYCPLDTFTRLVNQVVEPACQI

SEQ ID NO: 22 - protein, 413 aa, synthetic construct
  REGION 1..413: note = PHY-13789
SEAAPAGYHLQKAVILSRHGVRAPTKMTQLMRDVTPYTWPEWPVPLGYITPRGEHLVKLMGGFYRQYFQQQGLLSRDNCPTPDDVYVWTDVNQRTRKTGEAFLAGLAPECDLTIHHQSDIKQVDPLFHPLKAGICSMDKTQVQQAVEKQAGMPIDKLNQHYRAELALMSNVLNFPKSPYCQRHSGEQPCDFAQMFPSYLNISDDGNEVQLEGAVGLSSTLAEIFLLEYAQGMPVVAWGNIHSEQEWNDLLKLHNAYFDLMERTPYIAKHNGTPLLQTIVNALNPNATESKLPDISPDVKILFLAGHDTNIANIGGMLGMDWTLPGQPDNTPPGGALLFELWSDKEGTQYVSVKMVYQTLAQLRNMTPLTLKEPAGSVPLKIPGCDDQTAEGYCPLDTFTRLVNQVVEPACQLP

SEQ ID NO: 23 - protein, 413 aa, synthetic construct
  REGION 1..413: note = PHY-13798
SEAAPAGYHLQKAVILSRHGVRAPTKMTQTMRDVTPYTWPEWPVPLGYITPRGEHLVKLMGGFYRQYFQQQGLLSKDNCPTANDVYVWTDVAQRTRKTGEAFLAGLAPECDLTIHHQNNISQVDPLFHPVKAGICSMDKTQVQQAVEKQAGMPIDKLNQHYRPELALMSNVLNFPKSPYCQRHSGEKPCDFAQMMPSKLNISDDGNEVQLEGAVGLSSTLAEIFLLEYAQGMPVVAWGNIHSEQEWNDLLKLHNAHFDLMERTPYIAKHNGTPLLQAISNALNPNTTESKLPDISPDVKILFLAGHDTNIANIGGMLGMTWTLPGQPDNTPPGGALLFELWRDKAGTQYVSVHMVYQTLAQLRNMTPLTLKEPAGSVPLKIPGCDDQTAEGYCPLDTFTRLVNQVVEPACQLP

SEQ ID NO: 24 - protein, 413 aa, synthetic construct
  REGION 1..413: note = PHY-13868
SDTAPAGFQLEKVVILSRHGVRAPTKMTQLMRDVTPYTWPEWPVPLGYITPRGEHLISLMGGFYRQYFQQQGLLSKDNCPTANDVYVWTDVDQRTRKTGEAFLAGLAPECDLTIHHQSDIKQADPLFHPVKAGICSMDKSQTYEAVEKQAGGPIETLNQHYRPELALMSNVLNFPKSPYCRRHSGDKTCDFANAFPSYLNISDDGNEVQLEGAVGLSSTLAEIFLLEYAQGMPVVAWGNIHSEQEWNDLLKLHNAHFDLMQRTPYIAKHQGTPLLQTIVNALNSNTTARELPDISPDVKILFLAGHDTNIANIGGMLGMTWTLPGQPDNTPPGGALLFELWSDKEGTQYVSVEMVYQTLAQLRNMTPLTLKEPAGTVPLKIPGCDDQTAEGYCPLDTFTRLVNQVVEPACQLP

SEQ ID NO: 25 - protein, 413 aa, synthetic construct
  REGION 1..413: note = PHY-13883
SEAAPSGYQLEKVVILSRHGVRAPTKMTQTMRDVTPYTWPEWPVPLGYITPRGEHLVKLMGGFYRQYFQQQGLLSRDNCPTANDVYVWTDVDQRTRKTGEAFLAGLAPGCDLTIHHQNNISQADPLFHPVKAGICSMDKTQVQQAVEKQAGMPIDKLNQHYRAELALMSNVLNFPKSPYCRQHSVEQPCDFANAFPSYLNISDDGNEVQLEGAVGLSSTLAEIFLLEYAQGMPVVAWGNIHSEQEWNALLKLHNAHFDLMQRTPYIAKHNGTPLLQTIVNALNSNTTARELPDISPDVKILFLAGHDTNIANIGGMLGMTWTLPGQPDNTPPGGALLFELWRDKDGTQYVSVHMVYQTLAQLRNMTPLTLKEPAGSVPLKIPGCDDQTAEGYCPLDTFTRLVNQVVEPACQLP

SEQ ID NO: 26 - protein, 413 aa, synthetic construct
  REGION 1..413: note = PHY-13885
SEAAPSGYHLDKAVILSRHGVRAPTKMTQLMRDVTPYTWPEWPVPLGYITPRGEHLVSLMGGFYRQYFQQQGLLSRDRCPTANDVYVWTDVNQRTRKTGEAFLAGLAPECDLTIHHQNDIKQVDPLFHPLKAGICSMDKTQVQQAVEKQAGMPIDKLNQHYRPELALMSNVLNFPKSPYCRQHSVEQPCDFANAFPSYLNISDDGNEVQLEGAVGLSSTLAEIFLLEYAQGMPVVAWGNIHSEQEWNDLLKLHNAQFDLMERTPYIAKHQGTPLLQTIVNALNSNTTESKLPDISPSVKILFLAGHDTNIANIGGMLGMTWTLPGQPDNTPPGGALLFELWSDKDGTQYVSVKMVYQTLAQLRNMTPLTLKEPAGSVPLKIPGCDDQTAEGYCPLDTFTRLVNQVVEPACQLP

SEQ ID NO: 27 - protein, 413 aa, synthetic construct
  REGION 1..413: note = PHY-13936
SEAAPAGYHLDKAVILSRHGVRAPTKMTQTMRDVTPYTWPEWPVPLGYITPRGEHLVKLMGGFYRQYFQQQGLLPRDSCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCQRHSGEQPCDFANAFPSYLNISDDGNEVQLEGAVGLSSTLAEIFLLEYAQGMPVVAWGNIHSEQEWNALLKLHNAHFDLMERTPYIAKHNGTPLLQTIVNALNSNTTESKLPDISPSVKILFLAGHDTNIANIGGMLGMTWTLPGQPDNTPPGGALLFELWSDKEGTQYVSVEMVYQTLAQLRNQTPLTLKEPAGSVPLKIPGCDDQTAEGYCPLDTFTRLVNQVVEPACQLP

SEQ ID NO: 28 - protein, 412 aa, synthetic construct
  REGION 1..412: note = PHY-14004
AEEANGMKLQKAVILSRHGVRAPTKMTQLMRDVTPYTWPEWPVPLGYITPRGEHLVSLMGGFYRQYFQQQGLLSRDSCPTANDVYVWTDVNQRTRKTGEAFLAGLAPGCDLTIHHQNDIKQVDPLFHPLKAGICSMDKTQVQQAVEKQAGMPIDKLNQHYRPELALMSNVLNFPKSPYCQRHSGEQPCDFANMFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDVKILFLAGHDTNIANIAGMLGMTWTLPGQPDNTPPGGALLFELWSDKDGTQYVSVHMVYQTLAQLRNQTPLTLKEPAGSVPLKIPGCDDQTAEGYCPLDTFTRLVNQVVEPACQLP

SEQ ID NO: 29 - protein, 413 aa, synthetic construct
  REGION 1..413: note = PHY-14215
SEAAPAGYHLQKAVILSRHGVRAPTKMTQLMRDVTPYTWPEWPVPLGYITPRGEHLVSLMGGFYRQYFQQQGLLSRDSCPTANDVYVWTDVDQRTRKTGEAFLAGLAPGCDLAIHHQQNITQVDPLFHPLKAGICSMDKTQVQQAVEKQAGMPIDKLNQHYRPELALMSNVLNFPKSPYCRRHSGDKTCDFANAFPSYLNISDDGNKVALDGAVGLSSTLAEIFLLEYAQGMPVVAWGNIHSESQWKSLLNLHNAHFNLMQRTPYIAKHNGTPLLQTIVNALNPNATESKLPDISPDNKILFLAGHDTNIANIGGMLGMTWTLPGQPDNTPPGGALLFELWRDKEGTQYVSVEMVYQTLAQLRNQTPLTLKEPAGSVPLKIPGCDDQTAEGYCPLDTFTRLVNQVVEPACQLP

SEQ ID NO: 30 - protein, 413 aa, synthetic construct
  REGION 1..413: note = PHY-14256
SDTAPAGYQLDKAVILSRHGVRAPTKMTQLMRDVTPYTWPEWPVPLGYITPRGEHLVKLMGGFYRQYFQQQGLLSKDNCPTANDVYVWTDVNQRTRKTGEAFLAGLAPGCDLTIHHQNDIKQADPLFHPLKAGICSMDKTQVQQAVEKQAGMPIDKLNQHYRPELALMSNVLNFPKSPYCQRHSVDKPCDFANAFPSYLNISDDGNEVQLEGAVGLSSTLAEIFLLEYAQGMPVVAWGNIHSEQEWNDLLKLHNAYFDLMQRTPYIAKHQGTPLLQTIVNALNSNTTESKLPDISPDVKILFLAGHDTNIANIAGMLGMDWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 31 - protein, 412 aa, synthetic construct
  REGION 1..412: note = PHY-14277
SEAAPAGYHLDKAVILSRHGVRAPTKMTQLMRDVTPYTWPEWPVPLGYITPRGEHLVSLMGGFYRQYFQQQGLLPKDNCPTANDVYVWTDVNQRTRKTGEAFLAGLAPECDLTIHHQNDIKQVDPLFHPVKAGICSMDKTQVQQAVEKQAGMPIDKLNQHYRPELALMSNVLNFPKSPYCRRHSGDKTCDFANAFPSYLNISDDGNEVQLEGAVGLSSTLAEIFLLEYAQGMPVVAWGNIHSEQEWNDLLKLHNAQFDLMERTPYIAKHQGTPLLQTIVNALNSNTTESKLPDISPDVKILFLAGHDTNIANIGGMLGMTWTLPGQPDNTPPGGALLFELWSDKEGTQYVSVKMVYQTLAQLRNMTPLTLKEPAGTVPLKIPGCDDQTAEGYCPLDTFTRLVNQVVEPACQI

SEQ ID NO: 32 - protein, 412 aa, synthetic construct
  REGION 1..412: note = PHY-14473
AEEQNGMKLQKAVILSRHGVRAPTKMTQLMRDVTPYTWPEWPVPLGYITPRGEHLVSLMGGFYRQYFQQQGLLSRDRCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCRHQSGDKTCDFANAFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 33 - protein, 413 aa, synthetic construct
  REGION 1..413: note = PHY-14614
SEAAPSGYHLDKAVILSRHGVRAPTKMTQTMRDVTPYTWPEWPVPLGYITPRGEHLVSLMGGFYRQYFQQQGLLSRDSCPTANDVYVWTDVNQRTRKTGEAFLAGLAPGCDLTIHHQQNITQVDPLFHPVKAGICSMDKTQVQQAVEKQAGMPIDKLNQHYRPELALMSNVLNFPKSPYCQQHSVDKPCDFANMFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIAGMLGMDWTLPGQPDNTPPGGALLFELWSDKEGTQYVSVKMVYQTLAQLRNMTPLTLKEPAGTVPLKIPGCDDQTAEGYCPLDTFTRLVNQVVEPACQLP

SEQ ID NO: 34 - protein, 413 aa, synthetic construct
  REGION 1..413: note = PHY-14804
SDTAPAGFQLDKAVILSRHGVRAPTKMTQTMRDVTPYTWPEWPVPLGYITPRGEHLVSLMGGFYRQYFQQQGLLPRDNCPTANDVYVWTDVDQRTRKTGEAFLAGLAPECDLTIHHQSDIKQVDPLFHPLKAGICSMDKTQVQQAVEKQAGMPIDELNQHYRAELALMSNVLNFPKSPYCQRHSGEQPCDFANAFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDVKILFLAGHDTNIANIGGMLGMTWTLPGQPDNTPPGGALLFELWSDKDGTQYVSVKMVYQTLAQLRNQTPLTLKEPAGSVPLKIPGCDDQTAEGYCPLDTFTRLVNQVVEPACQLP

SEQ ID NO: 35 - protein, 413 aa, synthetic construct
  REGION 1..413: note = PHY-14945
SEAAPAGYHLDKAVILSRHGVRAPTKMTQTMRDVTPYTWPEWPVPLGYITPRGEHLVKLMGGFYRERFQQQGLLSRDSCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCQRHSGEQPCDFANAFPSKLNISDDGNEVQLEGAVGLSSTLAEIFLLEYAQGMPVVAWGNIHSEQEWNDLLKLHNAHFDLMERTPYIAKHNGTPLLQTIVNALNSNTTARELPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 36 - protein, 412 aa, synthetic construct
  REGION 1..412: note = PHY-15459
AEPQNGMKLDKAVILSRHGVRAPTKMTQTMRDVTPYTWPEWPVPLGYITPRGEHLVSLMGGFYRQYFQQQGILSKDRCPRANDVYVWTDVDQRTRKTGEAFLAGLAPQCPLTIHHQNDIKQVDPMFHPVKGGICSMDKTQVQQAVEKQAGMPIDKLNDHYRPELALMSAVLNFPASPYCQRHSGEQPCDFAQMMPSKLYITDDGNEVQLEGAVGLSSTLAEIFLLEYAQGMPVVAWGNIHSEQEWNALLKLHNAYFDLMYRTPYIAKHNGTPLLQTIVNALNSNTTARELPDISPDNRILFLAGHDTNIANIGGMLGMDWTLPGQPDNTPPGGALLFELWSDKEGTQYVSVHMVYQTLAQLRNQTPLTLQEPAGTVPLKIPSCDDQTAEGYCPLDTFTKLVNQVVEPACQIP

SEQ ID NO: 37 - protein, 413 aa, synthetic construct
  REGION 1..413: note = PHY-16513
SEAAPSGYHLDKAVILSRHGVRAPTKMTQTMRDVTPYTWPEWPVPLGYITPRGEHLVSLMGGFYRQYFQMQGLLSRDRCPNANDVYVWTDVDQRTRKTGEAFLRGLAPECDLTIHHQNDIKQVDPMFHPVKGGICSMDKTQVQQAVEKQAGMPIDKLNDHYRPELALMSAVLNFPASPYCQQHSVDKPCDFANAFPSKLNITDDGNEVALDGAVGLSSTLAEIFLLEYAQGMPVVAWGNIHNESQWKSLLNLHNAHFNLMHRTPYIAKHNGTPLLQTIVNALNSNTTARELPDISPSVKILFLAGHDTNIANIGGMLGMDWTLPGQPDNTPPGGALLFELWSDKDGTQYVSVKMVYQTLAQLRNQTPLTLNEPAGTVPLKIPSCDDQTAEGYCPLDTFTVLVNQVVEPACQIP